SEQUENCE LISTING (54 sequences)

SEQ ID NO: 1 - RNA, 22 nt, Homo sapien
uaacacugucugguaacgaugu

SEQ ID NO: 2 - RNA, 22 nt, Homo sapien
uaauacugucugguaaaaccgu

SEQ ID NO: 3 - RNA, 23 nt, Homo sapien
uaauacugccugguaaugaugac

SEQ ID NO: 4 - RNA, 22 nt, Homo sapien
uaacacugucugguaaagaugg

SEQ ID NO: 5 - RNA, 22 nt, Homo sapien
uaauacugccggguaaugaugg

SEQ ID NO: 6 - RNA, 22 nt, Homo sapien
uccuucauuccaccggagucug

SEQ ID NO: 7 - RNA, 21 nt, Homo sapien
aucacauugccagggauuucc

SEQ ID NO: 8 - RNA, 21 nt, Homo sapien
uucacaguggcuaaguuccgc

SEQ ID NO: 9 - RNA, 21 nt, Homo sapien
ggcaagaugcuggcauagcug

SEQ ID NO: 10 - RNA, 24 nt, Homo sapien
aaaagugcuuacagugcagguagc

SEQ ID NO: 11 - RNA, 22 nt, Homo sapien
uguaacagcaacuccaugugga

SEQ ID NO: 12 - RNA, 22 nt, Homo sapien
aacauucauugcugucgguggg

SEQ ID NO: 13 - RNA, 23 nt, Homo sapien
aacauucaacgcugucggugagu

SEQ ID NO: 14 - RNA, 21 nt, Homo sapien
uagcaccaucugaaaucgguu

SEQ ID NO: 15 - RNA, 22 nt, Homo sapien
uuaaugcuaaucgugauagggg

SEQ ID NO: 16 - RNA, 21 nt, Homo sapien
uucacaguggcuaaguucugc

SEQ ID NO: 17 - RNA, 22 nt, Homo sapien
cuggacuuggagucagaaggcc

SEQ ID NO: 18 - RNA, 22 nt, Homo sapien
aagcugccaguugaagaacugu

SEQ ID NO: 19 - RNA, 24 nt, Homo sapien
  misc_feature 4, 9, 11: N = C or U
uaanacugncncngguaannnngn

SEQ ID NO: 20 - RNA, 7 nt, Homo sapien
aacacug

SEQ ID NO: 21 - RNA, 7 nt, Homo sapien
aauacug

SEQ ID NO: 22 - RNA, 7 nt, Homo sapien
ccuucau

SEQ ID NO: 23 - RNA, 7 nt, Homo sapien
ucacauu

SEQ ID NO: 24 - RNA, 7 nt, Homo sapien
ucacagu

SEQ ID NO: 25 - RNA, 7 nt, Homo sapien
gcaagau

SEQ ID NO: 26 - RNA, 7 nt, Homo sapien
aaagugc

SEQ ID NO: 27 - RNA, 7 nt, Homo sapien
guaacag

SEQ ID NO: 28 - RNA, 7 nt, Homo sapien
acauuca

SEQ ID NO: 29 - RNA, 7 nt, Homo sapien
agcacca

SEQ ID NO: 30 - RNA, 7 nt, Homo sapien
uaaugcu

SEQ ID NO: 31 - RNA, 7 nt, Homo sapien
uggacuu

SEQ ID NO: 32 - RNA, 7 nt, Homo sapien
agcugcc

SEQ ID NO: 33 - DNA, 21 bp, Artificial Sequence Sequence
  Oligonucleotide Oligonucleotide primer
ttcaaacccatagtggttgct

SEQ ID NO: 34 - DNA, 21 bp, Artificial Sequence
  Oligonucleotide primer
tgggagataccaaaccaactg

SEQ ID NO: 35 - DNA, 18 bp, Artificial Sequence
  Oligonucleotide primer
caagaggcgcaaacaagc

SEQ ID NO: 36 - DNA, 20 bp, Artificial Sequence
  Oligonucleotide primer
ggttggcaataccgtcatcc

SEQ ID NO: 37 - DNA, 20 bp, Artificial Sequence
  Oligonucleotide primer
cggtccagaagaaatgaagg

SEQ ID NO: 38 - DNA, 21 bp, Artificial Sequence
  Oligonucleotide primer
tcctcaaagtctgatgtgcaa

SEQ ID NO: 39 - DNA, 19 bp, Artificial Sequence
  Oligonucleotide primer
cccaccacgtacaagggtc

SEQ ID NO: 40 - DNA, 19 bp, Artificial Sequence
  Oligonucleotide primer
ctggggtattgggggcatc

SEQ ID NO: 41 - DNA, 20 bp, Artificial Sequence
  Oligonucleotide primer
aagcggcctctacaacttca

SEQ ID NO: 42 - DNA, 20 bp, Artificial Sequence
  Oligonucleotide primer
aactgggaaatgtgagcacc

SEQ ID NO: 43 - DNA, 22 bp, Artificial Sequence
  Oligonucleotide primer
caacttgccagaaaactccagg

SEQ ID NO: 44 - DNA, 21 bp, Artificial Sequence
  Oligonucleotide primer
atgaaaccgggctatctgctc

SEQ ID NO: 45 - DNA, 21 bp, Artificial Sequence
  Oligonucleotide primer
atgaaaccgggctatcagctc

SEQ ID NO: 46 - DNA, 19 bp, Artificial Sequence
  Oligonucleotide primer
gcaactctgtggaccaagg

SEQ ID NO: 47 - DNA, 20 bp, Artificial Sequence
  Oligonucleotide primer
cactggcacgagagcttaaa

SEQ ID NO: 48 - DNA, 20 bp, Artificial Sequence
  Oligonucleotide primer
acccagaagactgtggatgg

SEQ ID NO: 49 - DNA, 19 bp, Artificial Sequence
  Oligonucleotide primer
catcactgccacccagaag

SEQ ID NO: 50 - DNA, 19 bp, Artificial Sequence
  Oligonucleotide primer
cagtgagcttcccgttcag

SEQ ID NO: 51 - DNA, 28 bp, Artificial Sequence
  Oligonucleotide primer
caactagtcaaaataaatccgggtgtgc

SEQ ID NO: 52 - DNA, 29 bp, Artificial Sequence
  Oligonucleotide primer
ttactagtacagcagttcaggcttgttga

SEQ ID NO: 53 - DNA, 28 bp, Artificial Sequence
  Oligonucleotide primer
atactagtggagttggagctgggtattg

SEQ ID NO: 54 - DNA, 30 bp, Artificial Sequence
  Oligonucleotide primer
acactagttggaatcaggatcagttgagaa

(19) United States
(12) Patent Application Publication
(10) Pub. No.: US 2010/0088775 A1
(43) Pub. Date: Apr. 8, 2010

(54) METHODS OF MODULATING EPITHELIAL-MESENCHYMAL TRANSITION AND MESENCHYMAL-EPITHELIAL TRANSITION IN CELLS AND AGENTS USEFUL FOR THE SAME

(75) Inventors: Yeesim Khew-Goodall, South Australia (AU); Greg Goodall, South Australia (AU)

Correspondence Address: KNOBBE MARTENS OLSON & BEAR LLP, 2040 MAIN STREET, FOURTEENTH FLOOR, IRVINE, CA, 92614, US

(73) Assignee: Medvet Science Pty. Ltd., Stepney, SA (AU)

(21) Appl. No.: 12/302,244

(22) PCT Filed: May 25, 2007
(86) PCT No.: PCT/AU07/00736

§ 371 Date: Jun. 23, 2009

Related U.S. Application Data
(60) Provisional application No. 60/808,735, filed on May 26, 2006.

(30) Foreign Application Priority Data
Sep. 13, 2006 (AU) 2006905081

(51) Int. Cl. A61K 39/395; A61K 31/7088; C12N 5/071; A01K 67/027; A61P 35/00; A61P 17/02

(52) U.S. Cl. 800/13; 424/172.1; 514/44 A; 435/366

(57) ABSTRACT

The present invention relates generally to the fields of treatment, prophylaxis and diagnosis of cell-based and fibrotic conditions in animals including mammals. More particularly, the present invention contemplates the use of agents which modulate epithelial-mesenchymal transition (EMT) processes and mesenchymal-epithelial transition (MET) processes and hence are useful in the treatment of a range of conditions including inhibiting metastasis of solid tumors and the development of fibrosis, treating metastatic disease and in promoting wound healing. Diagnostic protocols to assess EMT and MET or its stage of development also form part of the present invention. The EMT and MET modulating agents are also useful in regulating gene expression and, hence, represent useful therapeutic and research tools.

### FIELD OF THE INVENTION

The present invention relates generally to the fields of treatment, prophylaxis and diagnosis of cell-based and fibrotic conditions in animals including mammals. More particularly, the present invention contemplates the use of agents which modulate epithelial-mesenchymal transition (EMT) processes and mesenchymal-epithelial transition (MET) processes and hence are useful in the treatment of a range of conditions including inhibiting metastasis of solid tumors and the development of fibrosis, treating metastatic disease and in promoting wound healing. Diagnostic protocols to assess EMT and MET or its stage of development also form part of the present invention. The EMT and MET modulating agents are also useful in regulating gene expression and, hence, represent useful therapeutic and research tools.

### BACKGROUND OF THE INVENTION

Full bibliographic details of references cited herein are collected at the end of the subject specification.

The reference to any prior art in this specification is not, and should not be taken as, an acknowledgment or any form of suggestion that that prior art forms part of the common general knowledge in Australia.

Epithelial-mesenchymal transition (EMT) is a process whereby epithelial cells that are normally non-proliferative and non-mobile undergo transition into mesenchymal cells characterized by a proliferative and mobile phenotype. It is a central mechanism for diversifying cells found in complex tissue, hence, is a process involved in organizing the formulation of the body plan (Kalluri and Nelson J Clin Invest 112(12):1776-1784, 2003). Although epithelial cells were once considered to be terminally differentiated, it is recognized that epithelia possess an element of plasticity enabling transition to mobile mesenchymal cells (Boyer et al. Biochem Pharmacol 60:1099, 2000; Nieto Nat Rev Mol Cell Biol 3:155-166, 2002). EMT is required, therefore, in adult tissue to enable formation of fibroblasts in injured tissues (Strutz et al. J Cell Biol 130:393-405, 1995; Iwano et al. J Clin Invest 110:341-350, 2002) and in initiating, metastases in epithelial cancer (Kiermer et al. Oncogene 20:6679-6688, 2001; Janda et al. J Cell Biol 156:299-313, 2002; Xue et al. Cancer Res 63:3386-3394, 2003).

EMT is, therefore, a process of disaggregating epithelial units and re-shaping epithelia for movement in the formation of mesenchymal cells. The transition requires a molecular reprogramming of epithelium, generally considered to be by a variety of cytokines, metalloproteinases and membrane assembly inhibitors (Kalluri and Neilson 2003 supra; Yang and Liu Am J Pathol 159:1465-1475, 2001; Zeisberg et al. Am J Pathol 159:1313-1321, 2001; Fan Kindney Int 56:1455-1467, 1999). It is unclear, however, what regulates the EMT process at the genetic level.

There is a need, therefore, to elucidate the complex genetic regulatory mechanism in order to develop agents which are capable of regulating not just single factors, such as single cytokines, but groups or families of factors. This is particularly important to enable the development of agents which can assist in reducing metastasis of epithelial tumors, to control fibrosis and to promote wound healing.

MicroRNAs (miRNAs) are an abundant class of non-coding RNAs that have been associated with gene expression (Slack Science 287:1431-1433, 2000; Krützfeldt et al Nature Letters 438:685-1784, 2005). The miRNAs molecules are generally about 21 to 25 nucleotides in length and several hundred miRNAs have been identified to date. It is proposed that miRNAs repress expression of their target gene by interacting in a sequence-specific manner with a miRNA recognition motif on an mRNA transcript, thereby inhibiting protein translation from the mRNA and/or causing cleavage and degradation of the target mRNA.

In work leading up to the present invention, it has been determined that miRNAs are involved in controlling the EMT and MET processes and hence represent useful therapeutic and diagnostic targets.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, the term “derived from” shall be taken to indicate that a particular integer or group of integers has originated from the species specified, but has not necessarily been obtained directly from the specified source. Further, as used herein the singular forms of “a”, “and” and “the” include plural referents unless the context clearly dictates otherwise.

Unless otherwise defined, all technical and scientific terms used herein have the same meaning as commonly understood by one of ordinary skill in the art to which this invention belongs.

The subject specification contains nucleotide sequence information prepared using the programme PatentIn Version 3.1, presented herein after the bibliography. Each nucleotide sequence is identified in the sequence listing by the numeric indicator <210> followed by the sequence identifier (e.g. <210>1, <210>2, etc). The length, type of sequence (DNA, etc) and source organism for each sequence is indicated by information provided in the numeric indicator fields <211>, <212> and <213>, respectively. Nucleotide sequences referred to in the specification are identified by the indicator SEQ ID NO: followed by the sequence identifier (e.g. SEQ ID NO:1, SEQ ID NO:2, etc.). The sequence identifier referred to in the specification correlates to the information provided in numeric indicator field <400> in the sequence listing, which is followed by the sequence identifier (e.g. <400>1, <400>2, etc). That is SEQ ID NO:1 as detailed in the specification correlates to the sequence indicated as <400>1 in the sequence listing

One aspect of the present invention provides a method for modulating EMT said method comprising contacting an epithelial cell with an agent, which agent either elevates or reduces the functional level of one or more selected miRNAs or families of miRNAs.

In another aspect there is provided a method for modulating MET, said method comprising administering to a mesenchymal cell an agent, which agent either elevates or reduces the functional level of one or more selected miRNAs or families of miRNAs.

In yet another aspect the present invention contemplates a method for modulating EMT, said method comprising contacting an epithelial cell with an agent which either (i) elevates the functional level of an miRNA or family of miRNAs or (ii) reduces the functional level of an miRNA or family of miRNAs in epithelial or mesenchymal cells wherein said miRNAs are differentially expressed in either cell type in tissue undergoing EMT relative to epithelial tissue prior to, during or following EMT and wherein:
(i) upregulating the functional level of an miRNA which is downregulated in mesenchymal cells post EMT relative to epithelial cells, inhibits or down-regulates EMT;
(ii) downregulating the functional level of an miRNA which is downregulated in mesenchymal cells post EMT relative to epithelial cells induces or upregulates EMT;
(iii) upregulating the functional level of an miRNA which is upregulated in mesenchymal cells post EMT relative to epithelial cell induces or upregulates EMT; and
(iv) downregulating the functional level of an miRNA which is upregulated in mesenchymal cells post EMT relative to epithelial cells inhibits or down-regulates EMT.

Yet another aspect of the present invention contemplates a method for modulating MET, said method comprising administering to a mesenchymal cell an agent which either (i) elevates the functional level of an miRNA or family of miRNAs or (ii) reduces the functional level of an miRNA or family of miRNAs in epithelial or mesenchymal cells wherein said miRNAs are differentially expressed in either cell type in tissue undergoing MET relative to epithelial tissue prior to, during or following MET and wherein:
(i) upregulating the functional level of an miRNA which is downregulated in mesenchymal cells post EMT relative to epithelial cells induces or upregulates MET; and
(ii) downregulating the functional level of an miRNA which is upregulated in mesenchymal cells post EMT relative to epithelial cells induces or upregulates MET.

In still another aspect there is provided a method for downregulating or inhibiting EMT said method comprising contacting an epithelial cell with an agent which upregulates the functional level of one or more miRNAs or family of miRNAs or a functional analog thereof wherein said miRNA is upregulated in epithelial cells compared to mesenchymal cells following EMT.

In yet still another preferred embodiment there is provided a method for downregulating or inhibiting EMT said method comprising contacting an epithelial cell with an agent which downregulates the functional level of one or more miRNAs or family of miRNAs wherein said miRNA is downregulated in epithelial cells compared to mesenchymal cells following EMT.

In yet another aspect there is provided a method for upregulating EMT, said method comprising contacting an epithelial cell with an agent which upregulates the functional level of one or more miRNAs or family of miRNAs or a functional analog thereof wherein said miRNA is upregulated in mesenchymal cells compared to epithelial cells following EMT.

In still another aspect there is provided a method for upregulating MET, said method comprising contacting a mesenchymal cell with an agent which upregulates the functional level of one or more miRNAs or family of miRNAs wherein said miRNA is downregulated in mesenchymal cells compared to epithelial cells after EMT.

In yet another aspect there is provided a method for upregulating MET, said method comprising contacting a mesenchymal cell with an agent which downregulates the functional level of one or more miRNAs or family of miRNAs or a functional analog thereof wherein said miRNA is upregulated in mesenchymal cells compared to epithelial cells following EMT.

In another aspect of the present invention there is provided a method for modulating EMT said method comprising contacting an epithelial cell with an agent, which agent either elevates or reduces the functional levels of one or more selected miRNAs or families of miRNAs and which modulation results in the upregulation or downregulation of the expression of a gene carrying said miRNA recognition motif.

In a further aspect of the present invention there is provided a method for modulating MET said method comprising contacting a mesenchymal cell with an agent, which agent either elevates or reduces the functional levels of one or more selected miRNAs or families of miRNAs and which modulation results in the upregulation or downregulation of the expression of a gene carrying said miRNA recognition motif.

In yet another aspect of the present invention there is provided a method for upregulating EMT, said method comprising contacting an epithelial cell with an agent which targets an miRNA recognition motif to thereby prevent or reduce miRNA-mediated silencing of a gene comprising said miRNA recognition motif wherein said gene is characterised by an miRNA recognition motif which is targeted by a miRNA or family of miRNAs defined by any one or more of SEQ ID NOs:1-11 or 19.

Another aspect of the present invention is directed to a method for treating a subject, said method comprising administering to said subject an agent which either (i) elevates the functional level of an miRNA or family of miRNAs or functional fragment or derivative thereof or (ii) reduces the functional level of an miRNA or family of miRNAs or functional fragment or derivative thereof in epithelial or mesenchymal cells, which miRNAs are differentially expressed in either cell type in tissue undergoing EMT relative to epithelial tissue prior to, during or following EMT, and wherein:
(i) upregulating the functional level of an miRNA which is downregulated in mesenchymal cells post EMT relative to epithelial cells, inhibits or down-regulates EMT;
(ii) downregulating the functional level of an miRNA which is downregulated in mesenchymal cells post EMT relative to epithelial cells induces or upregulates EMT;
(iii) upregulating the functional level of an miRNA which is upregulated in mesenchymal cells post EMT relative to epithelial cell induces or upregulates EMT;
(iv) downregulating the functional level of an miRNA which is upregulated in mesenchymal cells post EMT relative to epithelial cells inhibits or down-regulates EMT;
(v) upregulating the functional level of an miRNA which is downregulated in mesenchymal cells post EMT relative to epithelial cells induces or upregulates MET; and
(vi) downregulating the functional level of an miRNA which is upregulated in mesenchymal cells post EMT relative to epithelial cells induces or upregulates MET.

In a further aspect there is provided a method for treating a subject by downregulating or inhibiting EMT, said method comprising administering to said subject an agent which upregulates the functional level of one or more miRNAs or family of miRNAs or functional fragment or derivative thereof wherein said miRNA is upregulated in epithelial cells compared to mesenchymal cells following EMT.

In still another further aspect there is provided a method for treating a subject by downregulating or inhibiting EMT, said method comprising administering to said subject an agent which downregulates the functional level of one or more miRNAs or family of miRNAs or functional fragment or derivative thereof wherein said miRNA is downregulated in epithelial cells compared to mesenchymal cells following EMT.

In yet still another further aspect there is provided a method for treating a subject by upregulating EMT, said method comprising administering to said subject an agent which downregulates the functional level of one or more miRNAs or family of miRNAs or functional fragment or derivative thereof wherein said miRNA is downregulated in mesenchymal cells compared to epithelial cells after EMT.

In yet another further aspect there is provided a method for treating a subject by upregulating EMT, said method comprising administering to said subject an agent which upregulates the functional level of one or more miRNAs or family of miRNAs or functional fragment or derivative thereof wherein said miRNA is upregulated in mesenchymal cells compared to epithelial cells following EMT.

In still another further aspect there is provided a method for treating a subject by upregulating MET, said method comprising administering to said subject an agent which upregulates the functional level of one or more miRNAs or family of miRNAs or functional fragment or derivative thereof wherein said miRNA is downregulated in mesenchymal cells compared to epithelial cells following EMT.

In yet another further aspect there is provided a method for treating a subject by upregulating MET, said method comprising administering to said subject an agent which downregulates the functional level of one or more miRNAs or family of miRNAs or functional fragment or derivative thereof wherein said miRNA is upregulated in mesenchymal cells compared to epithelial cells following EMT.

In yet another aspect of the present invention there is provided the use an agent capable of elevating or reducing miRNA levels in the manufacture of a medicament to modulate EMT in a subject.

Another aspect of the present invention is directed to the use of a population of cells treated in accordance with the method of the invention in the manufacture of a medicament for the treatment of a condition.

Yet another aspect of the present invention is directed to the use of an agent which either (i) elevates the functional level of an miRNA or family of miRNAs or functional fragment or derivative thereof or (ii) reduces the functional level of an miRNA or family of miRNAs or functional fragment or derivative thereof in epithelial or mesenchymal cells, which miRNAs are differentially expressed in either cell type in tissue undergoing EMT relative to epithelial tissue prior to, during or following EMT, in the manufacture of a medicament for the treatment of a condition wherein said agent modulates EMT wherein:

A list of sequence identifiers referred to herein is provided in Table 1.

TABLE 1
Summary of Sequence Identifiers

| miRNA | SEQ ID NO |
|---|---|
| has-miR-200a | 1 |
| has-miR-429 | 2 |
| has-miR-200b | 3 |
| has-miR-141 | 4 |
| has-miR-200c | 5 |
| has-miR-205 | 6 |
| has-miR-23a | 7 |
| has-miR-27a | 8 |
| has-miR-31 | 9 |
| has-miR-106a | 10 |
| has-miR-194 | 11 |
| has-miR-181b | 12 |
| has-miR-181a | 13 |
| has-miR-29a | 14 |
| has-miR-155 | 15 |
| has-miR-27b | 16 |
| has-miR-422b | 17 |
| has-miR-22 | 18 |
| consensus miRNA from SEQ ID NO: 1 through 5 | 19 |
| nucleotides 2-8 of SEQ ID NOs: 1 and 4 | 20 |
| nucleotides 2-8 of SEQ ID NOs: 2, 3 and 5 | 21 |
| nucleotides 2-8 of SEQ ID NO: 6 | 22 |
| nucleotides 2-8 of SEQ ID NO: 7 | 23 |
| nucleotides 2-8 of SEQ ID NOs: 8 and 16 | 24 |
| nucleotides 2-8 of SEQ ID NO: 9 | 25 |
| nucleotides 2-8 of SEQ ID NO: 10 | 26 |
| nucleotides 2-8 of SEQ ID NO: 11 | 27 |
| nucleotides 2-8 of SEQ ID NOs: 12 and 13 | 28 |
| nucleotides 2-8 of SEQ ID NO: 14 | 29 |
| nucleotides 2-8 of SEQ ID NO: 15 | 30 |
| nucleotides 2-8 of SEQ ID NO: 17 | 31 |
| nucleotides 2-8 of SEQ ID NO: 18 | 32 |

### BRIEF DESCRIPTION OF THE DRAWINGS

FIG. 1 is a schematic representation of the miR-200 family that are similar in sequence and are located together in the genome, on chromosome 1.

FIG. 2 (a) is an image of MDCK cells stably expressing empty vector (epithelial) or Pez (fibroblastoid). (b) is a graphical representation of qRT-PCR showing E-cadherin, Snail, ZEB1 and ZEB2 mRNA expression in Pez- vs Vector-MDCK clones. Data pooled from 3 vector and 4 Pez clones. p<0.05 (Student's t-test).

FIG. 3 is an image of Pez expression by whole mount in situ hybridisation. Lateral views; anterior to left. Arrow indicate in A brain VZ/SVZ 24hpf; inset dorsal view, B heart 42hpf, C pectoral fin 24hpf, D somites 24hpf.

FIG. 4 is an image of 96hpf embryos. Top panels: Horizontal sections through head, H&E stained; line shows shortened longitudinal axis through brain. Bottom panels: longitudinal view of trunk showing differences in somite boundary (short arrows) and pigmentation (long arrows).

FIG. 5 is a graphical representation of qRT-PCR showing Hey1 mRNA expression in Pez- vs Vector-MDCK clones. Data pooled from 3 vector and 4 Pez clones. p<0.05 (Student's t-test)

FIG. 6 is a schematic representation of 96hpf embryos. Top panels: Horizontal sections through head, H&E stained; line shows shortened longitudinal axis through brain. Bottom panels: longitudinal view of trunk showing differences in somite boundary (short arrows) and pigmentation (long arrows).

FIG. 7 is a graphical representation of LNA-PCR which allows specific quantitation of closely related microRNAs. LNA-PCR was performed on synthetic microRNA templates, as indicated.

FIG. 8 is a graphical representation of LNA-PCR showing miR-205 and members of the miR-200 family are strongly downregulated in MDCK cells that have undergone EMT. RNA was isolated from vector-MDCK (V) control polyclonal pool and two individual Pez-MDCK clones. MicroRNAs as shown were quantitated by LNA-PCR and mRNAs by conventional quantitative RF-PCR.

FIG. 9 is an image of a Western blot demonstrating anti-PY Ab of A431 epithelial cells transfected with vector or a dom. negative Pez (ΔPTP-Pez). Arrowheads show tyrosine phosphorylated proteins specific to ΔPTP-Pez transfectants.

FIG. 10 is a graphical representation demonstrating that overexpression of miR-200b downregulates its targets ZEB1 and ZEB2 resulting in upregulation of E-cadherin (which is repressed by ZEBs). These experiments were carried out in Pez-MDCK cells which exhibit a mesenchymal phenotype characterized by high ZEBs and low E-cadherin and low to negligible miR-200b.

FIG. 11 is an image demonstrating that overexpression of miR-200b in Pez-MDCK cells causes reversion to the epithelial phenotype, including change in shape from fibroblast-like cells (long spindly cells, top left panel) to epithelial-like cells (cuboidal cells, top right panel). Morphological change is accompanied by reorganisation of actin filaments from stress fibres (middle, left) to cortical actin surrounding the cells (middle, right) and increased E-cadherin and relocalisation to the cell-cell junctions (bottom, right). Cortical actin and junctional E-cadherin are typical features of epithelial cells.

FIG. 12 is a graphical representation demonstrating that miRNAs-200a, -200b and -205 transfected into MDA-MB-231 human breast cancer cells leads to downregulation of ZEB1 and ZEB2 mRNAs and upregulation of E-cadherin mRNA, indicative of a reversion of the invasive, dedifferentiated phenotype, to a more differentiated, less invasive, phenotype.

FIG. 13 is an image demonstrating that MDA-MB-231 human breast cancer cells transfected with miRs-200a, -200b and -205 undergo a shape change indicative of reversion to less invasive phenotype.

FIG. 14 is an image showing the morphology of MDCK and MDCK-Pez cells. Scale bars represent 100 μm. (b) is a graphical representation of a volcano plot showing changes in microRNAs detected by microarray of RNA from MDCK versus MDCK-Pez cells. Bayesian log odds of differential expression is plotted against log2[(expression in MDCK)/(expression in MDCK-Pez)] (c) is the representation of the sequence alignment of microRNAs of the miR-200 family. Nucleotides 2-7, representing the “seed” sequence, are underlined (d) is a representation of the chromosomal locations of the members of the miR-200 family in the human genome. The same clustering is found in other vertebrates, including the dog. (e) is a graphical representation of quantitation of microRNAs in MDCK and MDCK-Pez cells as measured by TaqMan real time PCR. PCRs were performed in triplicate with data pooled from 1-3 individual experiments ±s.e.m. (n=3−9).

FIG. 15 (a) is an image of phase contrast microscopy of MDCK cells treated with TGF-(31 over a 20 day period. Scale bars represent 200 μm. (b) is a graphical representation of changes in expression of epithelial and mesenchymal markers in MDCK cells treated with TGF-β as measured by real time RT-PCR. (c) is a graphical representation of changes in microRNA levels in the TGF-β-treated MDCK cells as measured by real time locked nucleic acid mediated PCR (miR-200a and miR-200b) or TaqMan PCR. Three independent time courses were performed; the data shown are from a single representative time course experiment measured in triplicate (±s.e.m.).

FIG. 16 (a) is a schematic of predicted miR-200a, miR-200b, and miR-205 sites in the ZEB1 and SIP1 3′UTRs. (b) is a schematic representation of the reporter constructs. RL-let-7 contains 3 artificial let-7 sites (Pillai et al. 2005 Science 309, 1573-1576). The let-7 microRNAs were expressed at similar levels in MDCK and MDCK-Pez cells (data not shown). (c) is a graphical representation of the Renilla luciferase reporter plasmids (RL-control, RL-ZEB1, RL-SIP1, and RL-let-7) were transiently transfected into MDCK, MDCK-vector, or MDCKPez cells, along with a firefly luciferase reporter (pGL3 control) for normalisation. The luciferase activities were measured after 48 h. Data are pooled from five experiments, with each transfection performed in triplicate, and are shown as the ratio of Renilla luciferase activity to firefly luciferase activity, ±s.e.m. (n=6-15). (d) is a graphical representation of MDCK-Pez cells cotransfected with the Renilla luciferase reporters and 4 nM of synthetic miR-200a, miR-200b and miR-205 precursors (Pre-miR, Ambion) either separately or in combination (all). The luciferase activities were measured after 48 h. Data are expressed relative to the activity in cells transfected with a negative control Pre-miR (neg) after normalising to pGL3 control to normalise transfection efficiency. The data are pooled from two experiments performed in Gregory et al. 12 triplicate with data measured ±s.e.m. (n=6). (e) is a graphical representation of MDCK cells cotransfected with the Renilla luciferase reporters and 30 nM of miR-200a, miR-200b and miR-205 inhibitors (AntimiR, Ambion) either separately or in combination (all). The luciferase activities were measured after 48 h. Data are expressed relative to the activity in cells transfected with a negative control Pre-miR (neg) after normalising to pGL3 control to normalise transfection efficiency. The data are pooled from two experiments performed in triplicate with data measured ±s.e.m. (n=6).

FIG. 17 is an image of phase contrast microscopy and E-cadherin or F-actin staining of MDCK cells transfected with a negative control or combination of miR-200a, miR-200b and miR-205 inhibitors (Anti-miR) for 9 days. DAPI staining was used to visualise nuclei and combined with the E-cadherin stained image in the merged panel. Scale bars represent 50 μm. (b) is a graphical representation of quantitation by real time PCR of EMT markers in MDCK cells transfected with microRNA inhibitors for 6 or 9 days. Inhibitors were transfected either separately or in combination (all) with the results expressed relative to a negative control Anti-miR (neg). The data are taken from a representative experiment of three transfection experiments and are shown ±s.e.m. (n=3). All values are normalised to GAPDH. (c) is a graphical representation of migration towards serum of MDCK cells transfected with negative control Anti-miR or a combination of Anti-miRs to miR-200a, miR-200b and miR-205. Data are pooled from triplicate migration measurements from duplicate transfections ±s.e.m. (n=6). (d) is an image of phase contrast microscopy and E-cadherin or F-actin staining of MDCK-Pez cells transfected with synthetic miR-200a, miR-200b and miR-205 precursors (Pre-miR) for 3 days. DAPI staining was used to visualise nuclei and combined with the E-cadherin stained image in the merged panel. Scale bars represent 10 μm. (e) is a graphical representation of quantitation by real time PCR of EMT markers in MDCK-Pez cells transfected with microRNA precursors for 3 days. MicroRNAs were transfected either separately or in combination (all), with the results expressed relative to a negative control Anti-miR (neg). The data are taken from three transfection experiments with qPCR performed in duplicate and are shown ±s.e.m. (n=6). All values are normalised to GAPDH. (f) is an image of a Western blot of ZEB1 and tubulin in cells transfected with Pre-miRs from the experiment above. For comparison, the levels of ZEB1 in MDCK and MDCK-Pez cells are shown.

FIG. 18 are images of phase contrast micrographs which show cell morphology of four well-characterised human breast cancer lines. Scale bars represent 100 μl m MicroRNAs and E-cadherin, ZEB1, and SIP1 mRNAs levels Gregory et al. 13 were measured by real time PCR. Data are pooled from a single experiment with measurements in triplicate and are shown ±s.e.m. (n=3).

FIG. 19 is a graphical representation of analysis of the ZEB1 mRNA using the UCSC genome browser (http://genome.ucsc.edu) which revealed truncation of the annotated Refseq sequence (NM_{—}030751). The probable terminus of the ZEB1 3′UTR is indicated by multiple expressed sequence tags (ESTs) ending at the same position (−1.2 kb downstream of the Refseq terminus).

FIG. 20 is a graphical representation of a range of concentrations of miR-200b Pre-miR cotransfected with RL-control or RL-ZEB1. Comparisons are made with samples without cotransfected microRNA (Con or ZEB1) or cotransfected with a negative control Pre-miR (neg). The pGL3 plasmid was cotransfected to normalise for transfection efficiency and the ratio of Renilla/firefly activity is shown from triplicate transfections ±s.e.m. (n=3).

### DETAILED DESCRIPTION OF THE INVENTION

The present invention is predicated in part on the identification of differentially expressed microRNAs (miRNAs) in epithelial cells or mesenchymal cells during epithelial-mesenchymal transition (EMT) and mesenchymal to epithelial transition (MET). DNA arrays identified miRNAs, including families of related miRNAs, which are either upregulated or downregulated in epithelial cells prior to EMT or in mesenchymal cells post EMT. As the miRNAs, including families of miRNAs, target mRNA from a range of genes, manipulating miRNA functional levels is proposed to enable the modulation of levels of a multiplicity of cytokines and other factors involved in EMT or MET. These determinations now permit the rational design of therapeutic and/or prophylactic methods for treating conditions which are characterised by EMT events such as wound healing, solid tumor metastisation, fibrosis, diabetic renal nephropathy, allograft dysfunction cataracts and defects in cardiac valve formation. There is also now facilitated diagnostic methodology to assess an individuals likelihood of EMT development or monitoring the state of EMT or MET in a subject.

Accordingly, one aspect of the present invention provides a method for modulating EMT said method comprising contacting an epithelial cell with an agent, which agent either elevates or reduces the functional level of one or more selected miRNAs or families of miRNAs.

In another aspect there is provided a method for modulating MET, said method comprising administering to a mesenchymal cell an agent, which agent either elevates or reduces the functional level of one or more selected miRNAs or families of miRNAs.

Without limiting the present invention to any one theory or mode of action “RNA interference” broadly describes a mechanism of gene silencing which is based on degrading or otherwise preventing the translation of mRNA in a highly sequence specific manner. In terms of the application of this technology to selectively knocking down gene expression, exogenous double stranded RNA (dsRNA) specific to the gene sought to be knocked down can be introduced into the intracellular environment. Once the dsRNA enters the cell, it is cleaved by an RNaseIII-like enzyme, Dicer, into double stranded small interfering RNAs (siRNAs) 21-23 nucleotides in length that contain 2 nucleotide overhangs on the 3′ ends. In an ATP dependent step, the siRNAs become integrated into a multi-subunit protein complex known as the RNAi induced silencing complex (RISC), which guides the siRNAs to the target RNA sequence. The siRNA unwinds and the antisense strand remains bound to RISC and directs degradation of the complementary target mRNA sequence by a combination of endo- and exonucleases. However, whereas the RNAi mechanism was originally identified in the context of its role as a microbial defence mechanism in higher eukaryotes, it is also known that RNAi based gene expression knockdown can also function as a mechanism to regulate endogenous gene expression. Specifically, microRNA (miRNA) is a form of endogenous single-stranded RNA which is typically 20-25 nucleotides and is endogenously transcribed from DNA, but not translated into protein. The DNA sequence that codes for an miRNA gene generally includes the miRNA sequence and an approximate reverse complement. When this DNA sequence is transcribed into a single-stranded RNA molecule, the miRNA sequence and its reverse-complement base pair to form a double stranded RNA hairpin loop, this forming the primary miRNA structure (pri-miRNA). A nuclear enzyme cleaves the base of the hairpin to form pre-miRNA. The pre-miRNA molecule is then actively transported out of the nucleus into the cytoplasm where the Dicer enzyme cuts 20-25 nucleotides from the base of the hairpin to release the mature miRNA.

Although both of the RNA interference mechanisms detailed above effectively achieve the same outcome, being selective gene expression knockdown, RNAi based on the use of exogenously administered dsRNA generally results in mRNA degradation while RNAi based on the actions of endogenous miRNAs generally results in translational repression by a mechanism which does not involve mRNA degradation. The RNA interference which is contemplated in the context of the present invention should be understood to encompass reference to both of these RNAi gene knockdown mechanisms. For example, although EMT been shown to be regulated by an endogenous miRNA based gene knockdown mechanism, the induction of this miRNA based knockdown mechanism could be achieved by administering, in accordance with the method of the invention, exogenous RNA oligonucleotides of the same sequence as the subject miRNA, pre-miRNA or pri-miRNA molecules. However, it should be understood that these exogenous RNA oligonucleotides may lead to either mRNA degradation (analogous to that observed with the introduction of an exogenous siRNA population) or mRNA translateral repression, this being akin to the mechanism by which the endogenous miRNA molecules function. In terms of the objective of the present invention, being the regulation of gene expression and, thereby, EMT, the occurrence of either gene knockdown mechanism is acceptable.

Without limiting the present invention to any one theory or mode of action, epithelial-mesenchymal transition (EMT) is an orchestrated series of events in which cell-cell and cell-extracellular matrix (ECM) interactions are altered to allow the release of epithelial cells from the surrounding tissue. The epithelial cell cytoskeleton is reorganised to confer the ability of the cell to move through a three-dimensional ECM via molecular reprogramming of the cell. Molecular reprogramming of an epithelial cell is necessary to achieve a mesenchymal phenotype and involves the downregulation of the expression of epithelial proteins, such as E-cadherin and junction proteins such as desmoplakin, claudin and occludin. In addition, the expression of mesenchymal proteins is upregulated, including for example, the expression of ECM proteins such as MMPS and fibronectin and cell surface proteins such as N-cadherin and integrin αvβ36. Transcription factors may also be upregulated in cells exhibiting a mesenchymal phenotype such as for example, snail, TWIST, ZEB1 (also known as δEF1) and ZEB2 (also known as SIP1). Reference to inducing the “transition” of an epithelial cell to a cell exhibiting a mesenchymal phenotype should be understood as a reference to inducing the genetic, morphologic and/or functional changes which are required to change an epithelial cell to a cell exhibiting a mesenchymal phenotype of the type defined herein. Reference to inducing mesenchymal to epithelial transition should be understood to have the converse meaning.

To this end reference to a cell which exhibits an “epithelial” or a “mesenchymal” cell phenotype should be understood as reference to a cell which exhibits one or more of the morphological, functional or structural characteristics which are exhibited by epithelial and mesenchymal cells, respectively, or mutants or variants thereof “Variants” include, but are not limited to, cells exhibiting some but not all of the morphological or phenotypic features or functional activities of epithelial cells at any differentiative stage of development. “Mutants” include, but are not limited to, epithelial cells which have been naturally or non-naturally modified such as cells which are genetically modified. An example of a variant or mutant of an epithelial cell or a mesenchymal cell is a cell which has become transformed or which has otherwise become neoplastic. As would be appreciated by the person of skill in the art, a neoplastic cell exhibits uncontrolled proliferative capacity, this usually being due to mutated gene functionality. Nevertheless, such mutated cells are recognisable as being epithelial or mesenchymal cells due to other functional or morphological characteristics which are typical of epithelial or mesenchymal cells, respectively. It should also be understood that the epithelial or mesenchymal cell may be at any differentiative stage of development. Without limiting the present invention to any one theory or mode of action, cells can develop an epithelial or mesenchymal phenotype relatively early in the differentiative process and maintain this phenotype through the process of differentiation along a particular somatic lineage. Accordingly, the subject cell may be either a mature cell of epithelial or mesenchymal phenotype or an immature cell of epithelial or mesenchymal phenotype.

Preferably, the subject cell is a cell of the breast, colon, stomach, small intestine, oesophagus, ovary, lung, kidney or prostate.

As previously detailed, it has been determined that coordination of the molecular programming in EMT involves regulation of the protein expression pattern, by miRNA, of epithelial cells prior to EMT and mesenchymal cells post EMT. Still further, it has also been determined that by reversing the miRNA expression levels which facilitate the EMT transition events, it is possible to induce mesenchymal transition back to an epithelial phenotype. As hereinafter described in more detail, this could be of particular significance in terms of reversing a neoplastic metastatic phenotype and thereby contributing to the treatment of a cancer.

The specific miRNAs of the present invention are molecules which are either elevated or reduced in epithelial cells prior to EMT or in mesenchymal cells post EMT. Accordingly, it should be understood that the present invention is directed to the modulation of EMT, by modulating the functional level of an miRNA or family of miRNAs in the subject epithelial or mesenchymal cells. Reference to “modulation of EMT” should be understood as a reference to inducing or preventing EMT or upregulating (elevating) or downregulating (reducing) the extent to which or rate at which this process occurs. Reference to a “functional level” of miRNA is a reference to the level functional miRNA and not necessarily the absolute level. For example, “reducing” the functional level of miRNA can be achieved either by reducing the absolute level of the subject miRNA or by rendering the miRNA non-functional, such as via the use of an antagonist. It would be appreciated that in this case there occurs a decrease in the functional level of the subject miRNA without necessarily reducing the absolute concentration of this molecule. Similarly, partial antagonism may act to reduce, although not necessarily eliminate, the functional effectiveness of the subject miRNA.

Reference to “elevating”, “upregulating”, “reducing” or “downregulating” miRNA functional levels includes both increasing and decreasing the number of miRNA molecules as well as increasing or decreasing the functionality of the miRNA molecules even if the number of miRNA molecules remains unchanged. Hence, decreasing the functional level of an miRNA includes, for example, reducing the ability of the molecule to interact with its miRNA recognition motif.

Particular miRNAs contemplated herein are defined in SEQ ID NOs:1 through 18.

The present invention also extends to families of miRNAs. One particular family is defined in SEQ ID NOs:1 through 5. Hence, the present invention extends to a family of miRNAs having the consensus nucleotide sequence set forth in SEQ ID NO:19:

(SEQ ID NO: 19)

UAAN₁ACUGN₂CN₂CN₃GGUAAN₄N₅N₆N₇G[N₈]ₙ

wherein
   N₁ is C or U;
   N₂ is C or U;
   N₃ is U or G;
   N₄ is C, U or A;
   N₅ is G or A;
   N₆ is A or C;
   N₇ is U, A or G;
   N₈ is G, A, U, C or G;
   n is o or 1.

The present invention also provides, therefore, an isolated miRNA, said miRNA being differentially expressed in epithelial cells and mesenchymal cells prior to, during or following EMT.

Accordingly, the present invention contemplates a method for modulating EMT, said method comprising contacting an epithelial cell with an agent which either (i) elevates the functional level of an miRNA or family of miRNAs or (ii) reduces the functional level of an miRNA or family of miRNAs in epithelial or mesenchymal cells wherein said miRNAs are differentially expressed in either cell type in tissue undergoing EMT relative to epithelial tissue prior to, during or following EMT and wherein:
(i) upregulating the functional level of an miRNA which is downregulated in mesenchymal cells post EMT relative to epithelial cells, inhibits or down-regulates EMT;
(ii) downregulating the functional level of an miRNA which is downregulated in mesenchymal cells post EMT relative to epithelial cells induces or upregulates EMT;
(iii) upregulating the functional level of an miRNA which is upregulated in mesenchymal cells post EMT relative to epithelial cell induces or upregulates EMT; and
(iv) downregulating the functional level of an miRNA which is upregulated in mesenchymal cells post EMT relative to epithelial cells inhibits or down-regulates EMT.

Yet another aspect of the present invention contemplates a method for modulating MET, said method comprising administering to a mesenchymal cell an agent which either (i) elevates the functional level of an miRNA or family of miRNAs or (ii) reduces the functional level of an miRNA or family of miRNAs in epithelial or mesenchymal cells wherein said miRNAs are differentially expressed in either cell type in tissue undergoing MET relative to epithelial tissue prior to, during or following MET and wherein:
(i) upregulating the functional level of an miRNA which is downregulated in mesenchymal cells post EMT relative to epithelial cells induces or upregulates MET; and
(ii) downregulating the functional level of an miRNA which is upregulated in mesenchymal cells post EMT relative to epithelial cells induces or upregulates MET.

Reference in this context to “wherein said miRNAs are differentially expressed in either cell type in tissue undergoing EMT relative to epithelial tissue prior to, during or following EMT” should be understood as a reference to the class of miRNAs which are suitable for modulation, in terms of their functional level, in order to modulate EMT or MET. Specifically, the miRNAs which may be targeted are those which are differentially expressed in epithelial cells prior to EMT versus the mesenchymal cells which result from EMT. More specifically, miRNAs which are downregulated in mesenchymal cells which have resulted from EMT, relative to epithelial cells prior to EMT, are useful for targetting in epithelial cells prior to or in the early stages of EMT. In particular, upregulating the functional levels of these miRNAs provides a means of inhibiting the EMT process while downregulating their levels provides a means of inducing EMT. The converse application is true for miRNAs which are upregulated in mesenchymal cells, which have resulted from EMT, relative to epithelial cells prior to EMT.

Preferably said miRNA or family of miRNAs is selected from SEQ ID NOs:1 through 19.

In one preferred embodiment there is provided a method for downregulating or inhibiting EMT said method comprising contacting an epithelial cell with an agent which upregulates the functional level of one or more miRNAs or family of miRNAs or a functional analog thereof wherein said miRNA is upregulated in epithelial cells compared to mesenchymal cells following EMT.

Preferably, said miRNAs are defined by SEQ ID NOs:1-11 or 19.

More preferably, said miRNAs are defined by SEQ ID NOs:1-5 or 19.

In another preferred embodiment, said miRNA is defined by SEQ ID NO 6.

In still another preferred embodiment there is provided a method for downregulating or inhibiting EMT said method comprising contacting an epithelial cell with an agent which downregulates the functional level of one or more miRNAs or family of miRNAs wherein said miRNA is downregulated in epithelial cells compared to mesenchymal cells following EMT.

Preferably, said miRNAs are defined by SEQ ID NOs:12-18.

In still another preferred embodiment there is provided a method for upregulating EMT, said method comprising contacting an epithelial cell with an agent which down-regulates the functional level of one or more miRNAs or family of miRNAs wherein said miRNA is downregulated in mesenchymal cells compared to epithelial cells after EMT.

Preferably, said miRNAs are defined by SEQ ID NOs:1-11 or 19.

More preferably, said miRNAs are defined by SEQ ID NOs:1-5 or 19.

In another preferred embodiment, said miRNA is defined by SEQ ID NO 6.

In yet another preferred embodiment there is provided a method for upregulating EMT, said method comprising contacting an epithelial cell with an agent which upregulates the functional level of one or more miRNAs or family of miRNAs or a functional analog thereof wherein said miRNA is upregulated in mesenchymal cells compared to epithelial cells following EMT.

Preferably, said miRNAs are defined by SEQ ID NOs:12-18.

In still another preferred embodiment there is provided a method for upregulating MET, said method comprising contacting a mesenchymal cell with an agent which upregulates the functional level of one or more miRNAs or family of miRNAs wherein said miRNA is downregulated in mesenchymal cells compared to epithelial cells after EMT.

Preferably, said miRNAs are defined by SEQ ID NOs:1-11 or 19.

More preferably, said miRNAs are defined by SEQ ID NOs:1-5 or 19.

In another preferred embodiment, said miRNA is defined by SEQ ID NO 6.

In yet another preferred embodiment there is provided a method for upregulating MET, said method comprising contacting a mesenchymal cell with an agent which down-regulates the functional level of one or more miRNAs or family of miRNAs or a functional analog thereof wherein said miRNA is upregulated in mesenchymal cells compared to epithelial cells following EMT.

Preferably, said miRNAs are defined by SEQ ID NOs:12-18.

Without limiting the present invention to any one theory or mode of action it is thought that miRNAs function to modulate EMT by regulating the gene expression of epithelial cells prior to EMT. The reverse process of mesenchymal-epithelial transition (MET) occurs by regulating the gene expression pattern of mesenchymal cells.

Without limiting the present invention to any one theory or mode of action, it is thought that two transcription factors ZEB1 (δEF1) and ZEB2 (SIP1) instigate EMT through their repression of the epithelial cell-cell adhesion protein E-cadherin. Both proteins have been determined to contain target sites (recognition sequences) for the miR-200a miRNA family and miR-205. Within the miR-200a family, miR200a and miR-141 have been found to interact with the same target site, while miR-200b, miR-200c and miR-429 interact with the same target site, both these target sites being different to one another. Still further, it has been found that the 3′ UTR of ZEB1 contains 2 binding sites for miR-200a, 5 for miR-200b and 1 for miR-205. The 3′ UTR of SIP1 contains 3 sites for miR-200a, 5 sites for miR-200b and 2 for miR-205.

To this end, a miRNA is complementary to a part of one or more messenger RNAs (mRNAs) these regions of interaction being referred to herein as “miRNA recognition motifs” or “miRNA recognition sequences”. Annealing of the miRNA to it's recognition motif on mRNA is thought to inhibit protein translation, due to the miRNA complex blocking the protein translation machinery or otherwise preventing protein translation without causing the mRNA to be degraded. However, it is possible that annealing of an miRNA to it's recognition motif facilitates cleavage of the mRNA. In the case of mRNA cleavage, without being bound by theory, the formation of the double-stranded RNA through the binding of the miRNA may trigger the degradation of the mRNA transcript through a process similar to RNA interference (RNAi) which is induced by siRNA molecules. miRNAs may also target methylation of genomic sites which correspond to targeted mRNAs.

Accordingly, the functions of miRNA to be interfered with can include functions such as translocation of RNA to a site of protein translation, translocation of RNA to sites within the cell which are distant from the site of RNA synthesis, translation of protein from an RNA, splicing of the RNA to yield one or more RNA species, and catalytic activity or complex formation involving the RNA which may be engaged in or facilitated by the RNA. Preferably, the result of such interference is the modulation of EMT or MET in a subject by either elevating or reducing the expression of genes carrying an miRNA recognition motif.

In another embodiment of the present invention there is provided a method for modulating EMT said method comprising contacting an epithelial cell with an agent, which agent either elevates or reduces the functional levels of one or more selected miRNAs or families of miRNAs and which modulation results in the upregulation or downregulation of the expression of a gene carrying said miRNA recognition motif.

In a further embodiment of the present invention there is provided a method for modulating MET said method comprising contacting a mesenchymal cell with an agent, which agent either elevates or reduces the functional levels of one or more selected miRNAs or families of miRNAs and which modulation results in the upregulation or downregulation of the expression of a gene carrying said miRNA recognition motif.

To the extent that the miRNAs of the present invention interact with regions of mRNA transcripts, it is thought that nucleotides 2-8, and preferably 2-7, of SEQ ID NOs:1-19 are particularly relevant to specifying the miRNA recognition motif. Accordingly, one may seek to modulate the functional level of the subject miRNAs by modulating the functionality (such as the availability for binding) of these regions. Nucleotide sequences 2-8 of SEQ ID NOs:1-18 are provided by SEQ ID NOs:20-32, these being detailed in Table 1.

Accordingly, in yet another embodiment of the present invention there is provided a method for upregulating EMT, said method comprising contacting an epithelial cell with an agent which targets an miRNA recognition motif to thereby prevent or reduce miRNA-mediated silencing of a gene comprising said miRNA recognition motif wherein said gene is characterised by an miRNA recognition motif which is targeted by a miRNA or family of miRNAs defined by any one or more of SEQ ID NOs:1-11 or 19.

Preferably said gene is characterised by an miRNA recognition motif defined by SEQ ID NOS: 20-27.

In yet another embodiment of the present invention there is provided a method for downregulating or inhibiting EMT, said method comprising contacting an epithelial cell with an agent which targets an miRNA recognition motif to thereby prevent or reduce miRNA-mediated silencing of a gene comprising said miRNA recognition motif wherein said gene is characterised by an miRNA recognition motif which is targeted by a miRNA or family of miRNAs defined by any one or more of SEQ ID NOs:12-18.

In still another embodiment of the present invention there is provided a method for upregulating or inducing MET, said method comprising contacting a mesenchymal cell with an agent which targets an miRNA recognition motif to thereby prevent or reduce miRNA-mediated silencing of a gene comprising said miRNA recognition motif wherein said gene is characterised by an miRNA recognition motif which is targeted by a miRNA or family of miRNAs defined by any one or more of SEQ ID NOs:12-18.

Preferably said gene is characterised by an miRNA recognition motif defined by SEQ ID NOS: 24 and 28-32.

In terms of inducing either the transition of the subject epithelial cell to a mesenchymal cell or the reverse, this can be achieved in vitro, such as in the context of small scale in vitro culture or large scale bioreactor production, or in an in vivo microenvironment. To the extent that the method is performed in vivo, the subject method is achieved by administering the subject agent to the patient in issue in order to achieve modulation of the functionality of the subject miRNAs and, thereby, modulation of EMT or MET.

Regulation of EMT or MET by modulation of miRNA functional levels is achieved by administering to a subject or to an in vitro culture system an agent which upregulates or downregulates the functional level of the subject miRNAs. Examples of agents which one might utilise include:
(i) RNA oligonucleotides which can induce an RNA interference mechanism which achieves the same functional outcome as the subject miRNAs. Accordingly, the “miRNA” as referenced in the claims should be understood as a reference to either endogenous miRNA or exogenous RNA oligonucleotides which can effectively mimic the activity of a miRNA (assuming that upregulation of miRNA levels is sought), such as would occur where synthetically generated siRNA molecules are used. This is discussed in more detail hereinafter;
(ii) agents which can interact with a miRNA recognition motif; and
(iii) other proteinaceous or non-proteinaceous agonists or antagonists of miRNAs or recognition motifs (e.g. antibodies which bind to these molecules).

Reference to an “RNA oligonucleotide” should therefore be understood as a reference to an RNA nucleic acid molecule which is double stranded or single stranded and is capable of either effecting the induction of an RNA interference mechanism directed to knocking down the expression of a gene targeted by the miRNAs of the present invention or downregulating or preventing the onset of such a mechanism by inhibiting the functioning of the endogenous miRNA molecules. In this regard, the subject oligonucleotide may be capable of directly modulating an RNA interference mechanism or it may require further processing, such as is characteristic of hairpin double stranded RNA which requires excision of the hairpin region, longer double stranded RNA molecules which require cleavage by dicer or precursor molecules such as pre-miRNA which similarly require cleavage. Accordingly, the subject oligonucleotide is designed to hybridise to either:
(i) an miRNA recognition motif as hereinbefore defined; or
(ii) an endogenous miRNA molecule.

Alternatively, the subject RNA molecule is designed to mimic the endogenous miRNA. The subject oligonucleotide may be naturally expressed, such as might occur with a cell which is expressing a miRNA, or it may be the result of the transfection of a cell with an expression vector which enables transcription of the oligonucleotide encoded by the subject vector. Still further, the cell may be actively expressing the oligonucleotide at the time that it is introduced to the cellular population or it may have expressed the oligonucleotide at an earlier time point but retains the oligonucleotide expression product intracellularly. The vector may also be designed to express the oligonucleotide in an inducible manner. In either case, the cell provides a source of the oligonucleotide. The subject oligonucleotide may be double stranded (as is typical in the context of effecting RNA interference) or single stranded (as may be the case if one is seeking only to produce a RNA oligonucleotide suitable for binding to an endogenously expressed miRNA or miRNA recognition motif in order to antagonise its activity). Examples of RNA oligonucleotides suitable for use in the context of the present invention include, but are not limited to:
long double stranded RNA (dsRNA)—these are generally produced as a result of the hybridisation of a sense RNA strand and an antisense RNA strand which are each separately transcribed by their own vector. Such double stranded molecules are not characterised by a hairpin loop. These molecules are required to be cleaved by an enzyme such as Dicer in order to generate short interfering RNA (siRNA) duplexes. This cleavage event preferably occurs in the cell in which the dsRNA is transcribed.
(ii) hairpin double stranded RNA (hairpin dsRNA)—these molecules exhibit a stem-loop configuration and are generally the result of the transcription of a construct with inverted repeat sequences which are separated by a nucleotide spacer region, such as an intron. These molecules are generally of longer RNA molecules which require both the hairpin loop to be cleaved off and the resultant linear double stranded molecules to be cleaved by Dicer in order to generate siRNA. This type of molecule has the advantage of being expressible by a single vector.
(iii) short interfering RNA (siRNA)—these can be synthetically generated or, recombinantly expressed by the promoter based expression of a vector comprising tandem sense and antisense strands each characterised by its own promoter and a 4-5 thymidine transcription termination site. This enables the generation of 2 separate transcripts which subsequently anneal. These transcripts are generally of the order of 20-25 nucleotides in length. Accordingly, these molecules require no further cleavage to enable their functionality in the RNAi pathway.
(iv) short hairpin RNA (shRNA)—these molecules are also known as “small hairpin RNA” and are similar in length to the siRNA molecules but with the exception that they are expressed from a vector comprising inverted repeat sequences of the 20-25 nucleotide RNA molecule, the inverted repeats being separated by a nucleotide spacer. Subsequently to cleavage of the hairpin (loop) region, there is generated a functional siRNA molecule.
(v) micro RNA/small temporal RNA (miRNA/stRNA)—miRNA and stRNA are generally understood to represent naturally occurring endogenously expressed molecules. Accordingly, although the design and administration of a molecule intended to mimic the activity of a miRNA will take the form of a synthetically generated or recombinantly expressed siRNA molecule, the method of the present invention nevertheless extends to the design and expression of oligonucleotides intended to mimic miRNA, pri-miRNA or pre-miRNA molecules by virtue of exhibiting essentially identical RNA sequences and overall structure. Such recombinantly generated molecules may be referred to as either miRNAs or siRNAs.
(vi) miRNAs which mediate spatial development (sdRNAs), the stress response (srRNAs) or cell cycle (ccRNAs).
(vii) RNA oligonucleotides designed to hybridise and prevent the functioning of endogenously expressed miRNA or stRNA or exogenously introduced siRNA. It would be appreciated that these molecules are not designed to invoke the RNA interference mechanism but, rather, prevent the upregulation of this pathway by the miRNA and/or siRNA molecules which are present in the intracellular environment. In terms of their effect on the miRNA to which they hybridise, this is reflective of more classical antisense inhibition.

It would be appreciated that the person of skill in the art can determine the most suitable RNA oligonucleotide for use in any given situation. For example, although it is preferable that the subject oligonucleotide exhibits 100% complementarity or identity to its target nucleic acid molecule, the oligonucleotide may nevertheless exhibit some degree of mismatch to the extent that hybridisation sufficient to induce an RNA interference response in a sequence specific manner is enabled. Accordingly, it is preferred that the oligonucleotide of the present invention comprises at least 70% sequence identity, more preferably at least 90% complementarity and even more preferably, 95%, 96%, 97%, 98% 99% or 100% sequence identity.

The term “identity” as used herein includes partial similarity as well as exact identity between compared sequences at the nucleotide level. The term “similarity” includes differences but where nucleic acid molecules are nevertheless related to each other at the structural, functional, biochemical and/or conformational levels.

Terms used to describe sequence relationships between two or more polynucleotides include “reference sequence”, “comparison window”, “sequence similarity”, “sequence identity”, “percentage of sequence similarity”, “percentage of sequence identity”, “substantially similar” and “substantial identity”. A “reference sequence” is at least 5, 6, 7, 8, 9, 10, 11 or 12 and frequently 15 to 18 and often at least 25 or above, such as 30 monomer units including 12, 13, 14, 15, 16, 17, 18, 19, 20, 21, 22, 23, 24, 25, 26, 27, 28, 29 and 30 monomer units, in length. Because two polynucleotides may each comprise (1) a sequence (i.e. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous nucleotides that is compared to a reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as for example disclosed by Altschul et al. (Nucl. Acids. Res. 25: 3389, 1997). A detailed discussion of sequence analysis can be found in Unit 19.3 of Ausubel et al. (In: Current Protocols in Molecular Biology, John Wiley & Sons Inc. 1994-1998).

The terms “sequence similarity” and “sequence identity” as used herein refer to the extent to which sequences are identical or functionally or structurally similar on a nucleotide-by-nucleotide basis over a window of comparison. Thus, a “percentage of sequence identity”, for example, is calculated by comparing two optimally aligned sequences over the window of comparison, determining the number of positions at which the identical nucleic acid base (e.g. A, U, C, G, I) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software. Similar comments apply in relation to sequence similarity. Here, “identity”, “similarity” and “homologues” may all be used to describe the relatedness between sequences.

In the context of the present invention, “hybridization” means the pairing of complementary strands of oligomeric agents. In the present invention, the preferred mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric agents. For example, adenine and thymine are complementary nucleobases which pair through the formation of hydrogen bonds. Hybridization can occur under varying circumstances.

“Complementary”, as used herein, refers to the capacity for precise pairing between two nucleobases of an oligomeric agent. For example, if a nucleobase at a certain position of an oligonucleotide (an oligomeric agent), is capable of hydrogen bonding with a nucleobase at a certain position of a target nucleic acid, said target nucleic acid being a DNA, RNA, or oligonucleotide molecule, then the position of hydrogen bonding between the oligonucleotide and the target nucleic acid is considered to be a complementary position. The oligonucleotide and the further DNA, RNA, or oligonucleotide molecule are complementary to each other when a sufficient number of complementary positions in each molecule are occupied by nucleobases which can hydrogen bond with each other. Thus, “specifically hybridizable” and “complementary” are terms which are used to indicate a sufficient degree of precise pairing or complementarity over a sufficient number of nucleobases such that stable and specific binding occurs between the oligonucleotide and a target nucleic acid.

Reference herein to a “low stringency” includes and encompasses from at least about 0 to at least about 15% v/v formamide and from at least about 1 M to at least about 2 M salt for hybridization, and at least about 1 M to at least about 2 M salt for washing conditions. Generally, low stringency is at from about 25-30° C. to about 42° C., such as 25, 26, 27, 28, 29, 30, 31, 32, 33, 34, 35, 36, 37, 38, 39, 40, 41 and 42° C. The temperature may be altered and higher temperatures used to replace formamide and/or to give alternative stringency conditions. Alternative stringency conditions may be applied where necessary, such as medium stringency, which includes and encompasses from at least about 16% v/v to at least about 30% v/v formamide, such as 16, 17, 18, 19, 20, 21, 22, 23, 24, 25, 26, 27, 28, 29 and 30% and from at least about 0.5 M to at least about 0.9 M salt, such as 0.5, 0.6, 0.7, 0.8 or 0.9 M for hybridization, and at least about 0.5 M to at least about 0.9 M salt, such as 0.5, 0.6, 0.7, 0.8 or 0.9 M for washing conditions, or high stringency, which includes and encompasses from at least about 31% v/v to at least about 50% v/v formamide, such as 31, 32, 33, 34, 35, 36, 37, 38, 39, 40, 41, 42, 43, 44, 45, 46, 47, 48, 49 and 50% and from at least about 0.01 M to at least about 0.15 M salt, such as 0.01, 0.02, 0.03, 0.04, 0.05, 0.06, 0.07, 0.08, 0.09, 0.10, 0.11, 0.12, 0.13, 0.14 and 0.15 M for hybridization, and at least about 0.01 M to at least about 0.15 M salt, such as 0.01, 0.02, 0.03, 0.04, 0.05, 0.06, 0.07, 0.08, 0.09, 0.10, 0.11, 0.12, 0.13, 0.14 and 0.15 M for washing conditions. In general, washing is carried out Tₘ=69.3+0.41 (G+C) % (Marmur and Doty, J. Mol. Biol. 5: 109, 1962). However, the Tₘ of a duplex DNA decreases by 1° C. with every increase of 1% in the number of mismatch base pairs (Bonner and Laskey, Eur. I Biochem. 46: 83, 1974). Formamide is optional in these hybridization conditions. Accordingly, particularly preferred levels of stringency are defined as follows: low stringency is 6×SSC buffer, 0.1% w/v SDS at 25-42° C.; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C.

In another example pertaining to the design of oligonucleotides suitable for use in the present invention, it is within the skill of the person of skill in the art to determine the particular structure and length of the subject oligonucleotide, for example whether it takes the form of dsRNA, hairpin dsRNA, siRNA, siRNA, miRNA, pre-miRNA, pri-miRNA etc. For example, it is generally understood that stem-loop RNA structures, such as hairpin dsRNA and shRNA, are more efficient in terms of achieving gene knockdown than, for example, double stranded DNA which is generated utilising two constructs separately coding the sense and antisense RNA strands. Still further, the nature and length of the intervening spacer region can impact on the functionality of a given stem-loop RNA molecule. In yet still another example, the choice of long dsRNA, which requires cleavage by an enzyme such as Dicer, or short dsRNA (such as siRNA or shRNA) can be relevant if there is a risk that in the context of the particular cellular environment an interferon response could be generated, this being a more significant risk where long dsRNA is used than where short dsRNA molecules are utilised. In still yet another example, whether a single stranded or double stranded nucleic acid molecule is required to be used will also depend on the functional outcome which is sought. For example, to the extent that one is targetting an endogenously expressed miRNA with an antisense molecule, it would generally be appropriate to design a vector which expresses a single stranded RNA oligonucleotide suitable for specifically hybridising to the subject miRNA. However, to the extent that it is sought to induce RNA interference, a double stranded siRNA molecule is required. This may be recombinantly expressed as a long dsRNA molecule which undergoes further cleavage or an siRNA, both of which can be produced from single or multiple vectors which are designed to express as separate transcripts the two complementary RNA oligonucleotides, or a hairpin long or short RNA molecule which can be expressed from a single vector as a single transcript. Still further, the present invention is preferably designed to result in the generation of a final effector RNA oligonucleotide (i.e. a siRNA or miRNA molecule) which is preferably less than 30 nucleotides in length, more preferably 15-25 nucleotides in length and most preferably 19, 20, 21, 22 or 23 nucleotides in length.

The preferred antagonistic agents are oligonucleotide-type agents which specifically hybridize to an miRNA, a region of an miRNA (such as that defined by SEQ ID NOs:20-32 or residues 1-7, preferably 1-6, of SEQ ID NOs:20-32), a consensus miRNA sequence or an miRNA recognition motif to thereby prevent its activity. Alternatively, the antagonistic agents are genetic constructs which encode an miRNA, are recombinant miRNAs or are synthetic miRNAs. For brevity, a genetic antagonist of an miRNA or miRNA-targeting agent is referred to as an “antagomer”. A genetic agonist is referred to as an “agomer” and includes constructs which encode an miRNA, are recombinant miRNAs or are synthetic miRNAs.

The term “antagomer” is used herein to define a genetic agent which inhibits or downregulates or otherwise reduces the function of a particular miRNA or family of miRNAs. Antagomers contemplated herein include anti-sense molecules, sense molecules (which induce co-suppression or RNAi-based silencing), ribozymes, double stranded RNA, modified RNAs (such as 2′O-methyl-RNA) and locked nucleic acids which selectively bind or target and inhibit the function of an miRNA. Antagomers also include synthetic and DNA-derived RNAi molecules or anti-sense molecules as well as constructs which produce these molecules. In addition, an antagomer of miRNA includes a molecule which masks or inhibits binding of an miRNA to an miRNA recognition motif such as an antibody or other proteinaceous or non-proteinaceous molecule.

An agent which elevates levels of an miRNA or a family of miRNAs is referred to herein as a “agomer”. Examples of agomers include genetic constructs which, under appropriate conditions, encodes a functionally effective region of the miRNA such as that defined by SEQ ID NOs:20-32 or residues 1-7, preferably 1-6, of SEQ ID NOs:20-32. Genetic constructs include recombinant virus expression systems, insect expression systems and eukaryotic cell expression systems. An agomer also includes functionally active synthetic miRNA molecules.

Particularly preferred antagomers are anti-sense or sense-type oligonucleotides which induce post-transcriptional silencing or which mask miRNA interaction with its recognition motif. However, it should be understood, as herein before described, that reference to an antagomer of miRNA also includes an antibody or other proteinaceous or non-proteinaceous molecule. Preferred agomers are DNA-derived or synthetic miRNAs. The present invention employs, therefore, agents, preferably oligonucleotides and similar species for use in modulating the function or effect of an miRNA. This is accomplished by providing oligonucleotides or analogues, or chemically modified forms thereof, which specifically hybridize to one or more miRNAs or their respective recognition sequences or which are complementary to either sequence or which functionally mimic an miRNA. The hybridization of an antagomer of the present invention with its target miRNA is generally referred to as “anti-sense” leading to anti-sense inhibition. Without limiting the present invention to any one theory or mode of action, such anti-sense inhibition is typically based upon hydrogen bonding-based hybridization of oligonucleotide strands or segments such that at least one strand or segment is rendered inoperable. Alternatively, a sense molecule having substantially the same sequence as a target miRNA or its recognition motif is employed to reduce either sense inhibition (referred to as co-suppression or RNAi-mediated inhibition) or which act to elevate miRNA levels. Chemically modified synthetic miRNA molecules are particularly useful. Additionally, hybridization of modified RNA such as 2′O-methyl RNA and locked nucleic acids leads to the formation of a stable complex which acts to block miRNA function.

It is understood in the art that the sequence of an anti-sense or sense antagomer or agomer need not be 100% complementary or identical to that of its target nucleic acid. It is preferred that the anti-sense or sense agents of the present invention comprise at least 70% sequence complementarity or identity to a target region within the target nucleic acid, more preferably that they comprise 90% sequence complementarity or identity and even more preferably comprise 95% sequence complementarity or identity to the target region within the target nucleic acid sequence.

According to the present invention, antagomers and agomers include anti-sense and sense oligonucleotides, ribozymes, external guide sequence (EGS) oligonucleotides, alternate splicers, primers, probes, and other oligomeric agents which hybridize to or are identical with at least a portion of the target miRNA or its recognition sequence. As such, these agents may be introduced in the form of single-stranded, double-stranded, circular or hairpin oligomeric agents and may contain structural elements such as internal or terminal bulges or loops. Once introduced to a system, the agents of the subject invention may elicit the action of one or more enzymes or structural proteins to effect modification of the target nucleic acid. Synthetic forms of the anti-sense and sense molecules may also be introduced.

Hence, the present invention comprehends other families of agents as well, including but not limited to oligonucleotide analogs and mimetics such as those described herein.

The agents in accordance with the present invention preferably comprise from about 7 to about 80 nucleobases. One of ordinary skill in the art will appreciate that the present invention embodies agents of 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleobases in length.

In another preferred embodiment, the agents of the invention are 12 to 50 nucleobases in length. One having ordinary skill in the art will appreciate that this embodies agents of 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleobases in length.

In yet another preferred embodiment, the agents of the invention are 15 to 30 nucleobases in length. One having ordinary skill in the art will appreciate that this embodies agents of 15, 16, 17, 18, 19, 20, 21, 22, 23, 24, 25, 26, 27, 28, 29, or 30 nucleobases in length.

Other preferred agents are oligonucleotides from about 12 to about 50 nucleobases, even more preferably those comprising from about 15 to about 30 nucleobases.

“Targeting” an anti-sense or sense agent to a particular miRNA or its recognition sequence, in the context of the present invention, can be a multistep process. The process usually begins with the identification of a target miRNA whose function is to be modulated or the recognition sequence of an miRNA in a gene whose expression is to be modulated. The targeting process usually also includes determination of at least one target region, segment, or site within the target miRNA or recognition motif for the anti-sense or sense interaction to occur such that the desired effect, e.g., modulation of expression, will result. Within the context of the present invention, the term “region” is defined as a portion of the target nucleic acid having at least one identifiable structure, function, or characteristic. Within regions of target nucleic acids are segments. “Segments” are defined as smaller or sub-portions of regions within a target nucleic acid. “Sites”, as used in the present invention, are defined as positions within a target nucleic acid.

In a further embodiment, the “preferred target segments” identified herein may be employed in a screen for additional agents which modulate the expression of the miRNA or a gene carrying an miRNA recognition sequence. “Modulators” are those agents which decrease or increase the expression of a nucleic acid molecule encoding an miRNA or a gene carrying an miRNA recognition motif and which comprise at least a 8-nucleobase portion which is complementary or identical to a preferred target segment. The screening method comprises the steps of contacting a preferred target segment of a nucleic acid molecule with one or more candidate modulators, and selecting for one or more candidate modulators which decrease or increase the expression of a nucleic acid molecule encoding the miRNA or a gene carrying a respective recognition sequence. Once it is shown that the candidate modulator or modulators are capable of modulating (e.g. either decreasing or increasing) the expression or activity of a target nucleic acid molecule, the modulator may then be employed in further investigative studies or for use as a research, diagnostic, or therapeutic agent in accordance with the present invention.

Anti-sense and sense agents of the present invention include oligonucleotides containing modified backbones or non-natural internucleoside linkages herein. These include chemically modified oligonucleotides or oligonucleotide analogs. Oligonucleotides having modified backbones include those that retain a phosphorus atom in the backbone and those that do not have a phosphorus atom in the backbone. For the purposes of the present specification, and as sometimes referenced in the art, modified oligonucleotides that do not have a phosphorus atom in their internucleoside backbone can also be considered to be oligonucleosides.

Useful modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, each of which is herein incorporated by reference.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, each of which is herein incorporated by reference.

In other oligonucleotide mimetics contemplated herein include both the sugar and the internucleoside linkage (i.e. the backbone), of the nucleotide units replaced with novel groups. One such agent, an oligonucleotide mimetic is referred to as a peptide nucleic acid (PNA). In PNA agents, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA agents include, but are not limited to, U.S. Pat. Nos. 5,539,082, 5,714,331 and 5,719,262, each of which is herein incorporated by reference. Further teaching of PNA agents can be found in Nielsen et al. Science 254:1497-1500, 1991.

Oligonucleotides with phosphorothioate backbones and oligonucleosides with heteroatom backbones, and in particular —CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂— [wherein the native phosphodiester backbone is represented as —O—P—O—CH₂—] and the amide backbones are also contemplated by the present invention.

Modified oligonucleotides may also contain one or more substituted sugar moieties.

Examples of such oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙ O]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other preferred oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al. Helv Chim Acta 78:486-504, 1995) i.e., an alkoxyalkoxy group. A further preferred modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Other useful modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. A particular 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, each of which is herein incorporated by reference in its entirety.

A further modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is conveniently a methylene (—CH₂—)ₙ group bridging the 2′ oxygen atom and the 4′ carbon atom wherein n is 1 or 2. LNAs and preparation thereof are described in WO 98/39352 and WO 99/14226.

Oligonucleotides may also include nucleobase modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 30: 613, 1991, and those disclosed by Sanghvi, Y. S., Chapter 15, Anti-sense Research and Applications, pages 289-302, Crooke, S. T. and Lebleu, B., ed., CRC Press, 1993. Certain of these nucleobases are particularly useful for increasing the binding affinity of the agents of the invention. These include 5-substituted pyrimidines, 6-azapyrimidines and N-2, N-6 and O-6 substituted purines, including 2-aminopropyl-adenine, 5-propynyluracil and 5-propynylcytosine. 5-methylcytosine substitutions have been shown to increase nucleic acid duplex stability by 0.6-1.2° C. and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. Nos. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,750,692; 5,587,469; 5,594,121, 5,596,091; 5,614,617; 5,645,985; 5,830,653; 5,763,588; 6,005,096; and 5,681,941, each of which is herein incorporated by reference.

Another modification of the oligonucleotides of the invention involves chemically linking to the oligonucleotide one or more moieties or conjugates which enhance the activity, cellular distribution or cellular uptake of the oligonucleotide. These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the agents of the present invention. Representative conjugate groups are disclosed in International Patent Application PCT/US92/09196, and U.S. Pat. No. 6,287,860, the disclosure of which are incorporated herein by reference. Conjugate moieties include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethyl-ammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety. Oligonucleotides of the invention may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethacin, a barbiturate, a cephalosporin, a sulfa drug, an antidiabetic, an antibacterial or an antibiotic.

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

It is not necessary for all positions in a given agent to be uniformly modified, and in fact more than one of the aforementioned modifications may be incorporated in a single agent or even at a single nucleoside within an oligonucleotide.

It should be understood that the terms “agent”, “chemical agent”, “agent”, “pharmacologically active agent”, “medicament”, “active” and “drug” are used interchangeably herein to refer to a chemical agent that induces a desired pharmacological and/or physiological effect such as modulating levels of miRNAs or their function or otherwise modulating EMT or MET processes.

The terms also encompass pharmaceutically acceptable and pharmacologically active ingredients of those active agents specifically mentioned herein including but not limited to salts, esters, amides, prodrugs, active metabolites, analogs and the like. When the terms “agent”, “chemical agent”, “agent”, “pharmacologically active agent”, “medicament”, “active” and “drug” are used, then it is to be understood that this includes the active agent per se as well as pharmaceutically acceptable, pharmacologically active salts, esters, amides, prodrugs, metabolites, analogs, etc. The aforementioned agents include genetic molecules termed “antagomers” and “agomers” which specifically modulate the level, function or availability (herein also referred to as the “functional level”) of miRNAs. Hence, the agents contemplated herein may be useful in genetic therapy. Insofar as the agent is a genetic molecule, it may be DNA, RNA, an anti-sense molecule, a sense molecule, double stranded or single stranded RNA or DNA, short interfering RNA (siRNA), RNA interference (RNAi), a complex of a nucleic acid and a ribonuclease or a chimera of a nucleic acid and another molecule.

The present invention still further enables genetic modification of genes to introduce or delete miRNA recognition motifs which will affect the ability of miRNAs to modulate the expression of the genes. Means for identifying, introducing or deleting miRNA recognition motifs would be well known to those skilled in the art.

Antagomers and agomers may be DNA-derived and, hence, expressed in a cell. Cells may further be engineered to express miRNA-encoding sequence to control expression of target genes.

To the extent the one seeks to genetically modify a cell to express the RNA oligonucleotide of interest, it would be appreciated that this can be achieved by any one of a number of methods which would be well known to the person of skill in the art. By “recombinantly engineer”, “recombinantly manipulate” and “genetically modify” is meant that the subject cell has undergone some form of molecular manipulation relative to that which is observed in the context of the majority of a corresponding unmodified population. Such modifications include, but are not limited to, the introduction of homologous or heterologous nucleic acid material to the cell. For example, the cell is rendered transgenic via the introduction of a DNA molecule encoding a RNA oligonucleotide or all or part of one or more genes. This arguably occurs in the context of the transfection of a nucleic acid molecule encoding an miRNA or corresponding to a promoter or other regulatory sequence. Preferably, the cell is transfected with a nucleic acid molecule encoding an RNA oligonucleotide. Even more preferably, the cell is permanently transfected DNA encoding the subject oligonucleotide. However, cells may be generated which transiently express a nucleic acid molecule encoding the oligonucleotide. This may be useful in certain circumstances where, for example, it is only sought to express the RNA oligonucleotide for a limited period of time. In another example, rather than transfecting a nucleic acid molecule encoding the RNA oligonucleotide into the cell, an endogenous but unexpressed genomic gene can be switched on, that is, expression of the gene is induced or even upregulated where the gene is either not expressed or not expressed in sufficiently high levels.

In addition to modification of these cells to express the RNA oligonucleotides of interest, other genes relevant to optimising the generation of the subject cells may also be introduced, including genes encoding marker proteins such as EGFP. Selection markers, such as antibiotic resistance genes (for example G418 resistance gene which enables the selection of mammalian cells using the neomycin analogue G418 or puromycin resistance gene), provide a convenient means of selecting for successful transformants while the incorporation of a suicide gene, such as the pMC1-thymidine kinase gene, facilitates the elimination of the introduced genetically modified cells subsequently to conclusion of the treatment regime. Again, this may be relevant where one is seeking to transiently modulate gene expression.

Reference to a “nucleic acid” should be understood as a reference to both deoxyribonucleic acid and ribonucleic acid thereof. The subject nucleic acid molecule may be any suitable form of nucleic acid molecule including, for example, a genomic, cDNA or ribonucleic acid molecule. To this end, the term “expression” refers to the transcription and translation of DNA or the translation of RNA resulting in the synthesis of a peptide, polypeptide or protein. A DNA construct, for example, corresponds to the construct which one may seek to transfect into a cell for subsequent expression while an example of an RNA construct is the RNA molecule transcribed from a DNA construct, which RNA construct merely requires translation to generate the protein of interest. Reference to “expression product” is a reference to the product produced from the transcription or translation of a nucleic acid molecule. In terms of the present invention, it would be appreciated that one is primarily seeking to effect transcription of an RNA oligonucleotide. However, transcription and translation may be required for molecules such as selective markers.

The term “protein” should be understood to encompass peptides, polypeptides and proteins. It should also be understood that these terms are used interchangeably herein. The protein may be glycosylated or unglycosylated and/or may contain a range of other molecules fused, linked, bound or otherwise associated to the protein such as lipids, carbohydrates or other peptides, polypeptides or proteins (such as would occur where a protein of interest is produced as a fusion protein with another molecule, for example GST or EGFP). Reference hereinafter to a “protein” includes a protein comprising a sequence of amino acids as well as a protein associated with other molecules such as amino acids, lipids, carbohydrates or other peptides, polypeptides or proteins.

It would be appreciated by the person of skill in the art that the mechanism by which these genetic modifications are introduced may take any suitable form which would be well known and understood by those of skill in the art. For example, genetic material is generally conveniently introduced to cells via the use of an expression construct. Alternatively, one may seek to use, as the starting cellular population, a cell type which either naturally or as a result of earlier random or directed genetic manipulation is characterised by one or more of the genetic modifications of interest.

Most preferably, said genetic modification is the transfection of a cell, which is sought to be treated, with an expression construct comprising one or more DNA regions comprising a promoter operably linked to a sequence encoding an RNA oligonucleotide, preferably a shRNA, and, optionally, a second DNA region encoding a selectable marker.

As detailed hereinbefore, the subject promoter may be constitutive or inducible. Where the subject construct expresses more than one molecule of interest, such as separate sense and antisense RNAs, these may be under the control of separate promoters. Where more than one shRNA is produced, such as two selection markers, these may also be under the control of separate promoters or a single promoter, such as occurs in the context of a bicistronic vector which makes use of an IRES sequence to facilitate the translation of more than one protein product, in an unfused form, from a single RNA transcript.

Reference to a nucleic acid “expression construct” should be understood as a reference to a nucleic acid molecule which is transmissible to a cell and designed to undergo transcription. The RNA molecule is then transcribed therefrom. In general, expression constructs are also referred to by a number of alternative terms, which terms are widely utilised interchangeably, including “expression cassette” and “vector”.

The expression construct of the present invention may be generated by any suitable method including recombinant or synthetic techniques. To this end, the subject construct may be constructed from first principles, as would occur where an entirely synthetic approach is utilised, or it may be constructed by appropriately modifying an existing vector. Where one adopts the latter approach, the range of vectors which could be utilised as a starting point are extensive and include, but are not limited to:
(i) Plasmids
   Plasmids are small independently replicating pieces of cytoplasmic DNA, generally found in prokaryotic cells, which are capable of autonomous replication. Plasmids are commonly used in the context of molecular cloning due to their capacity to be transferred from one organism to another. Without limiting the present invention to any one theory or mode of action, plasmids can remain episomal or they can become incorporated into the genome of a host. Examples of plasmids which one might utilise include the bacterial derived pBR322 and pUC.
(ii) Bacteriophage
   Bacteriophages are viruses which infect and replicate in bacteria. They generally consist of a core of nucleic acid enclosed within a protein coat (termed the capsid). Depending on the type of phage, the nucleic acid may be either DNA (single or double stranded) or RNA (single stranded) and they may be either linear or circular. Phages may be filamentous, polyhedral or polyhedral and tailed, the tubular tails to which one or more tubular tail fibres are attached. Phages can generally accommodate larger fragments of foreign DNA than, for example, plasmids. Examples of phages include, but are not limited to the E. coli lambda phages, P1 bacteriophage and the T-even phages (e.g. T4).
(iii) Baculovirus
   These are any of a group of DNA viruses which multiply only in invertebrates and are generally classified in the family Baculoviridae. Their genome consists of double-stranded circular DNA.
(iv) Artificial Chromosomes
   Artificial chromosomes such as yeast artificial chromosomes or bacterial artificial chromosomes.
(v) Hybrid vectors such as cosmids, phagemids and phasmids
   Cosmids are generally derived from plasmids but also comprise cos sites for lambda phage while phagemids represent a chimaeric phage-plasmid vector. Phasmids generally also represent a plasmid-phage chimaera but are defined by virtue of the fact that they contain functional origins of replication of both. Phasmids can therefore be propagated either as a plasmid or a phage in an appropriate host strain.
(vi) Commercially available vectors which are themselves entirely synthetically generated or are modified versions of naturally occurring vectors, such as the pENTR/V6 vector and pLenti6/BLOCK-iT-DEST expression construct.

It would be understood by the person of skill in the art that the selection of an appropriate vector for modification, to the extent that one chooses to do this rather than synthetically generate a construct, will depend on a number of factors. For example, where the cells are to be administered, or modified in vivo, into a human, it may be less desirable to utilise an RNA oligonucleotide expressing vector which is viral in nature. Further, it is necessary to consider the amount of DNA which is sought to be introduced to the construct. It is generally understood that certain vectors are more readily transfected into certain cell types. For example, the range of cell types which can act as a host for a given plasmid may vary from one plasmid type to another. In still yet another example, the larger the DNA insert which is required to be inserted, the more limited the choice of vector from which the expression construct of the present invention is generated. To this end, the size of the inserted DNA can vary depending on factors such as the size of the DNA sequence encoding any marker proteins of interest, the number of selection markers which are utilised and the incorporation of features such as linearisation polylinker regions and the like. As would be appreciated, the DNA encoding the RNA oligonucleotides of interest is itself quite small due to the small size of molecules such as shRNA.

The expression construct which is used in the present invention may be of any form including circular or linear. In this context, a “circular” nucleotide sequence should be understood as a reference to the circular nucleotide sequence portion of any nucleotide molecule. For example, the nucleotide sequence may be completely circular, such as a plasmid, or it may be partly circular, such as the circular portion of a nucleotide molecule generated during rolling circle replication (this may be relevant, for example, where a construct is being initially replicated, prior to its introduction to a cell population, by this type of method rather than via a cellular based cloning system). In this context, the “circular” nucleotide sequence corresponds to the circular portion of this molecule. A “linear” nucleotide sequence should be understood as a reference to any nucleotide sequence which is in essentially linear form. The linear sequence may be a linear nucleotide molecule or it may be a linear portion of a nucleotide molecule which also comprises a non-linear portion such as a circular portion. An example of a linear nucleotide sequence includes, but is not limited to, a plasmid derived construct which has been linearised in order to facilitate its integration into the chromosomes of a host cell or a construct which has been synthetically generated in linear form. To this end, it should also be understood that the configuration of the construct of the present invention may or may not remain constant. For example, a circular plasmid-derived construct may be transfected into a cell where it remains a stable circular episome which undergoes replication and transcription in this form. However, in another example, the subject construct may be one which is transfected into a cell in circular form but undergoes intracellular linearisation prior to chromosomal integration. This is not necessarily an ideal situation since such linearisation may occur in a random fashion and potentially cleave the construct in a crucial region thereby rendering it ineffective.

The nucleic acid molecules which are utilised in the method of the present invention are derivable from any human or non-human source. Non-human sources contemplated by the present invention include primates, livestock animals (e.g. sheep, pigs, cows, goats, horses, donkeys), laboratory test animal (e.g. mice, hamsters, rabbits, rats, guinea pigs), domestic companion animal (e.g. dogs, cats), birds (e.g. chicken, geese, ducks and other poultry birds, game birds, emus, ostriches) captive wild or tamed animals (e.g. foxes, kangaroos, dingoes), reptiles, fish, insects, prokaryotic organisms or synthetic nucleic acids.

It should be understood that the constructs of the present invention may comprise nucleic acid material from more than one source. For example, whereas the construct may originate from a bacterial plasmid, in modifying that plasmid to introduce the features defined herein nucleic acid material from non-bacterial sources may be introduced. These sources may include, for example, viral DNA, mammalian DNA (e.g. the DNA encoding an miRNA) or synthetic DNA (e.g. to introduce specific restriction endonuclease sites). Still further, the cell type in which it is proposed to express the subject construct may be different again in that it does not correspond to the same organism as all or part of the nucleic acid material of the construct. For example, a construct consisting of essentially bacterial and viral derived DNA may nevertheless be expressed in the mammalian cells contemplated herein.

A nucleic acid sequence encoding an miRNA or a complementary form thereof or a nucleic acid molecule engineering to carry an miRNA recognition sequence or have this sequence deleted may be introduced into a cell in a vector such that the nucleic acid sequence remains extrachromosomal (ectopic). In such a situation, the nucleic acid sequence will be expressed by the cell from the extrachromosomal location. Alternatively, cells may be engineered by inserting the nucleic acid sequence into the chromosome. Vectors for introduction of nucleic acid sequence both for recombination and for extrachromosomal maintenance are known in the art and any suitable vector may be used. Methods for introducing nucleic acids into cells such as electroporation, calcium phosphate co-precipitation and viral transduction are known in the art.

In particular, a number of viruses have been used as nucleic acid transfer vectors or as the basis for preparing nucleic acid transfer vectors, including papovaviruses (e.g. SV40, Madzak et al, J Gen Virol 73:1533-1536, 1992), adenovirus (Berkner, Curr Top Microbiol Immunol 158:39-66, 1992; Berkner et al, BioTechniques 6:616-629, 1988; Gorziglia and Kapikian, J Virol 66:4407-4412, 1992; Quantin et al, Proc Natl Acad Sci USA 89:2581-2584, 1992; Rosenfeld et al, Cell 68:143-155, 1992; Wilkinson et al, Nucleic Acids Res 20:233-2239, 1992; Stratford-Perricaudet et al, Hum Gene Ther 1:241-256, 1990; Schneider et al, Nat Genetics 18:180-183, 1998), vaccinia virus (Moss, Curr Top Microbiol Immunol 158: 5-38, 1992; Moss, Proc Natl Acad Sci USA 93:11341-11348, 1996), adeno-associated virus (Muzyczka, Curr Top Microbiol Immunol 158:97-129, 1992; Ohi et al, Gene 89:279-282, 1990; Russell and Hirata, Nat Genetics 18:323-328, 1998), herpesviruses including HSV and EBV (Margolskee, Curr Top Microbiol Immunol 158:67-95, 1992; Johnson et al, J Virol 66:2952-2965, 1992; Fink et al, Hum Gene Ther 3:1-19, 1992; Breakefield and Geller, Mol Neurobiol 1:339-371, 1987; Freese et al, Biochem Pharmaco. 40:2189-2199, 1990; Fink et al, Aim Rev Neurosci 19:265-287, 1996), lentiviruses (Naldini et al, Science 272:263-267, 1996), Sindbis and Semliki Forest virus (Berglund et al, Biotechnology 11:916-920, 1993) and retroviruses of avian (Bandyopadhyay and Temin, Mol Cell Biol 4:749-754, 1984; Petropoulos et al, J Virol 66:3391-3397, 1992), murine (Miller, Curr Top Microbiol Immunol 158:1-24, 1992; Miller et al, Mol Cell Biol 5:431-437, 1985; Sorge et al, Mol Cell Biol 4:1730-1737, 1984; Mann and Baltimore, J Virol 54:401-407, 1985; Miller et al, J Virol 62:4337-4345, 1988) and human (Shimada et al, J Clin Invest 88:1043-1047, 1991; Helseth et al, J Virol 64:2416-2420, 1990; Page et al, J Virol 64:5270-5276, 1990; Buchschacher and Panganiban, J Virol 66:2731-2739, 1982) origin.

Non-viral nucleic acid transfer methods are known in the art such as chemical techniques including calcium phosphate co-precipitation, mechanical techniques, for example, microinjection, membrane fusion-mediated transfer via liposomes and direct DNA uptake and receptor-mediated DNA transfer. Viral-mediated nucleic acid transfer can be combined with direct in vivo nucleic acid transfer using liposome delivery, allowing one to direct the viral vectors to particular cells. Alternatively, the retroviral vector producer cell line can be injected into particular tissue. Injection of producer cells would then provide a continuous source of vector particles.

It should also be understood that although the method of the present invention is exemplified with respect to in vitro cellular culture, this method may also be performed in vivo.

As would be appreciated, there is now provided means of routinely and reliably producing cells which have undergone EMT or MET transition on either a small in vitro scale or on a larger in vitro scale. This can be useful for generating cellular populations for subsequent use In terms of small scale production, which may be effected in tissue culture flasks for example, this may be particularly suitable for producing populations of cells for a given individual and in the context of a specific condition. One means of achieving large scale production in accordance with the method of the invention is via the use of a bioreactor.

Bioreactors are designed to provide a culture process that can deliver medium and oxygenation at controlled concentrations and rates that mimic nutrient concentrations and rates in vivo. Bioreactors have been available commercially for many years and employ a variety of types of culture technologies. Of the different bioreactors used for mammalian cell culture, most have been designed to allow for the production of high density cultures of a single cell type and as such find use in the present invention. Typical application of these high density systems is to produce as the end-product, a conditioned medium produced by the cells. This is the case, for example, with hybridoma production of monoclonal antibodies and with packaging cell lines for viral vector production. However, these applications differ from applications where the therapeutic end-product is the harvested cells themselves, as may occur in the present invention.

Once operational, bioreactors provide automatically regulated medium flow, oxygen delivery, and temperature and pH controls, and they generally allow for production of large numbers of cells. Bioreactors thus provide economies of labor and minimization of the potential for mid-process contamination, and the most sophisticated bioreactors allow for set-up, growth, selection and harvest procedures that involve minimal manual labor requirements and open processing steps. Such bioreactors optimally are designed for use with a homogeneous cell mixture or aggregated cell populations as contemplated by the present invention. Suitable bioreactors for use in the present invention include but are not limited to those described in U.S. Pat. No. 5,763,194, U.S. Pat. Nos. 5,985,653 and 6,238,908, U.S. Pat. No. 5,512,480, U.S. Pat. Nos. 5,459,069, 5,763,266, 5,888,807 and 5,688,687.

With any large volume cell culture, several fundamental parameters require almost constant control. Cultures must be provided with the medium as well as final cell culture/preservation. Typically, the various media are delivered to the cells by a pumping mechanism in the bioreactor, feeding and exchanging the medium on a regular basis. The exchange process allows for by-products to be removed from the culture. Growing cells or tissue also requires a source of oxygen. Different cell types can have different oxygen requirements. Accordingly, a flexible and adjustable means for providing oxygen to the cells is a desired component.

Depending on the particular culture, even distribution of the cell population and medium supply in the culture chamber can be an important process control. Such control is often achieved by use of a suspension culture design, which can be effective where cell-to-cell interactions are not important. Examples of suspension culture systems include various tank reactor designs and gas-permeable plastic bags. For cells that do not require assembly into a three-dimensional structure or require proximity to a stromal or feeder layer such suspension designs may be used. Also contemplated are 3 dimensional cultures which utilise a range of biological and synthetic scaffolds.

Efficient collection of the cells at the completion of the culture process is an important feature of an effective cell culture system. One approach for production of cells as a product is to culture the cells in a defined space, without physical barriers to recovery, such that simple elution of the cell product results in a manageable, concentrated volume of cells amenable to final washing in a commercial, closed system cell washer designed for the purpose. Optimally, the system would allow for addition of a pharmaceutically acceptable carrier, with or without preservative, or a cell storage agent, as well as providing efficient harvesting into appropriate sterile packaging. Optimally the harvest and packaging process may be completed without breaking the sterile barrier of the fluid path of the culture chamber.

With any cell culture procedure, a major concern is sterility. When the product cells are to be transplanted into patients (often at a time when the patient is ill or immunocompromised), absence of microorganisms is mandated. An advantage of the present cell production device over manual processes is that, as with many described bioreactor systems, once the culture is initiated, the culture chamber and the fluid pathway is maintained in a sterile, closed environment.

The cells generated in accordance with the method of the invention and agents used therein are useful for therapy, research and diagnostics.

Accordingly, a further aspect of the present invention relates to the use of the invention in relation to the treatment and/or prophylaxis of conditions which would benefit from modulation of the EMT process. For example, the regulation of EMT is an essential requirement in terms of controlling the transition of normally non-mobile epithelial cells to a mobile mesenchymal phenotype both in terms of normal physiology and in the context of many unwanted pathologies. For example, under normal physiological conditions at various stages during embryological development, transition of epithelial cells to a mesenchymal phenotype allows such cells to travel to distant regions in the embryo where they differentiate and/or induce differentiation of other cells to form the precursors of various tissues. In another example, EMT can also occur in adult tissues during wound repair, where it enables the formation of fibroblasts and tissue remodelling in injured tissue.

Inappropriate or unwanted induction of EMT can occur, however, during chronic inflammation or conditions that promote sustained tissue disruption which can stimulate fibrosis, thereby compromising tissue integrity and organ function. A further example of adverse EMT is that exhibited by cancer cells which undergo this process and thereby become metastatic due to their ability to separate from neighbouring cells and penetrate into and through surrounding tissues. EMT can also adversely function to aid cancer progression by providing increased resistance to apoptotic agents and by producing supporting tissues that enhance the malignancy of the central cancer.

Reference to “cancer” should be understood to include a tumor and encompasses for example epithelial tumors such as but not limited to tumors of the breast, colon, lung, ovary, pancreas and gastric region which includes for example, the stomach and oesophagus.

Accordingly, another aspect of the present invention is directed to a method for treating a subject, said method comprising administering to said subject an agent which either (i) elevates the functional level of an miRNA or family of miRNAs or functional fragment or derivative thereof or (ii) reduces the functional level of an miRNA or family of miRNAs or functional fragment or derivative thereof in epithelial or mesenchymal cells, which miRNAs are differentially expressed in either cell type in tissue undergoing EMT relative to epithelial tissue prior to, during or following EMT, and wherein:
(i) upregulating the functional level of an miRNA which is downregulated in mesenchymal cells post EMT relative to epithelial cells, inhibits or down-regulates EMT;
(ii) downregulating the functional level of an miRNA which is downregulated in mesenchymal cells post EMT relative to epithelial cells induces or upregulates EMT;
(iii) upregulating the functional level of an miRNA which is upregulated in mesenchymal cells post EMT relative to epithelial cell induces or upregulates EMT;
(iv) downregulating the functional level of an miRNA which is upregulated in mesenchymal cells post EMT relative to epithelial cells inhibits or down-regulates EMT;
(v) upregulating the functional level of an miRNA which is downregulated in mesenchymal cells post EMT relative to epithelial cells induces or upregulates MET; and
(vi) downregulating the functional level of an miRNA which is upregulated in mesenchymal cells post EMT relative to epithelial cells induces or upregulates MET.

In one preferred embodiment there is provided a method for treating a subject by downregulating or inhibiting EMT, said method comprising administering to said subject an agent which upregulates the functional level of one or more miRNAs or family of miRNAs or functional fragment or derivative thereof wherein said miRNA is upregulated in epithelial cells compared to mesenchymal cells following EMT.

Preferably, said miRNAs are defined by SEQ ID NOs:1-11 or 19.

More preferably, said miRNAs are defined by SEQ ID NOs:1-5 or 19.

In another preferred embodiment, said miRNA is defined by SEQ ID NO 6.

In still another preferred embodiment there is provided a method for treating a subject by downregulating or inhibiting EMT, said method comprising administering to said subject an agent which downregulates the functional level of one or more miRNAs or family of miRNAs or functional fragment or derivative thereof wherein said miRNA is downregulated in epithelial cells compared to mesenchymal cells following EMT.

Preferably, said miRNAs are defined by SEQ ID NOs:12-18.

In still another preferred embodiment there is provided a method for treating a subject by upregulating EMT, said method comprising administering to said subject an agent which downregulates the functional level of one or more miRNAs or family of miRNAs or functional fragment or derivative thereof wherein said miRNA is downregulated in mesenchymal cells compared to epithelial cells after EMT.

Preferably, said miRNAs are defined by SEQ ID NOs:1-11 or 19.

More preferably, said miRNAs are defined by SEQ ID NOs:1-5 or 19.

In another preferred embodiment, said miRNA is defined by SEQ ID NO 6.

In yet another preferred embodiment there is provided a method for treating a subject by upregulating EMT, said method comprising administering to said subject an agent which upregulates the functional level of one or more miRNAs or family of miRNAs or functional fragment or derivative thereof wherein said miRNA is upregulated in mesenchymal cells compared to epithelial cells following EMT.

Preferably, said miRNAs are defined by SEQ ID NOs:12-18.

In still another preferred embodiment there is provided a method for treating a subject by upregulating MET, said method comprising administering to said subject an agent which upregulates the functional level of one or more miRNAs or family of miRNAs or functional fragment or derivative thereof wherein said miRNA is downregulated in mesenchymal cells compared to epithelial cells following EMT.

Preferably, said miRNAs are defined by SEQ ID NOs:1-11 or 19.

More preferably, said miRNAs are defined by SEQ ID NOs:1-5 or 19.

In another preferred embodiment, said miRNA is defined by SEQ ID NO 6.

In yet another preferred embodiment there is provided a method for treating a subject by upregulating MET, said method comprising administering to said subject an agent which downregulates the functional level of one or more miRNAs or family of miRNAs or functional fragment or derivative thereof wherein said miRNA is upregulated in mesenchymal cells compared to epithelial cells following EMT.

Preferably, said miRNAs are defined by SEQ ID NOs:12-18.

“Treating” a subject may involve prevention of a condition or other adverse physiological event in a susceptible individual as well as treatment of a clinically symptomatic individual by ameliorating the symptoms of the condition. It includes reference to both therapeutic and prophylactic treatment. In particular, modulating EMT or MET is useful in preventing or reducing metastasis of solid epithelial tumors, reducing fibrosis of, for example, the lung and the kidney, promoting wound healing, modulating EMT related differentiation of stem cells, modulating organogenesis and preventing or reducing diseases and pathologies involving EMT such as diabetic renal nephropathy, allograft dysfunction, cataracts, or defects in cardiac valve formation. More specifically, inhibition of transition of epithelial cells to mesenchymal cells is desired in the treatment of cancer (by preventing the transition to metastatic disease), fibrotic diseases, diabetic renal nephropathy, allograft dysfunction, cataracts and defects in cardiac valve formation. Promotion of EMT, on the other hand, is desired in the promotion of wound healing and regeneration of tissues. Promotion of MET is desired in the context of treating metastatic tumours. Hence, both localised and systemic modulation of EMT and MET are contemplated by the present invention.

For therapeutics, a subject suspected of having a condition, disease or disorder associated with EMT such as metastatic or non-metastatic cancer, fibrosis, poor wound healing diabetic renal nephropathy, allograft dysfunction, cataracts or defects in cardiac valve formation can be treated by modulating the expression of a gene comprising an miRNA recognition motif treated by administering the agents of the present invention. For example, in one non-limiting embodiment, the method comprises the step of administering to the animal in need of treatment, a therapeutically effective amount of an antagomer or agomer. The antagomers or agomers of the present invention effectively inhibit the activity of or replicate the activity of an miRNA or family of miRNAs. The method of the invention should also be understood to extend to methods of treatment based on administering to a patient an effective number of cells, such as mesenchymal cells, which have been generated in accordance with the method of the invention. This may be particularly useful where one is seeking to increase the overall cell number of the population of cells in issue, such as mesenchymal cells in context of wound healing. In particular, the present invention provides a mechanism for treating a patient's own cells in vitro and thereafter reintroducing a syngeneic population of cells.

The term “subject” is used herein including humans, primates, livestock animals (e.g. horses, cattle, sheep, pigs, donkeys), laboratory test animals (e.g. mice, rats, guinea pigs), companion animals (e.g. dogs, cats) and captive wild animals (e.g. kangaroos, deer, foxes). Preferably, the mammal is a human or a laboratory test animal. Even more preferably, the mammal is a human.

Reference to “administering” to an individual the subject cells should be understood to include reference to either introducing into the mammal an ex vivo population of said cells which have been treated and/or differentiated according to the method of the invention or introducing into the mammal an effective amount of an agent which will act on an epithelial cell or mesenchymal cell which is either naturally present in the patient or has been administered in an untransitioned state. This latter situation may occur where a cell line is created using nuclear material derived from the patient in issue. In this regard, it may be desirable to treat the cell in accordance with the method of the invention ex vivo, for example in order to effect its expansion, but to conduct the actual step of either inducing transition or preventing the onset of transition, in the in vivo, and even more preferably in situ, environment.

In accordance with this aspect of the invention, the subject cells are preferably autologous cells which are treated ex vivo and transplanted back into the individual from which they were originally harvested. However, it should be understood that the present invention nevertheless extends to the use of cells derived from any other suitable source where the subject cells exhibit the same major histocompatability profile as the individual who is the subject of treatment. Accordingly, such cells are effectively autologous in that they would not result in the histocompatability problems which are normally associated with the transplanting of cells exhibiting a foreign MHC profile. Such cells should be understood as falling within the definition of “autologous”. For example, under certain circumstances it may be desirable, necessary or of practical significance that the subject cells are isolated from a genetically identical twin, or from an embryo generated using gametes derived from the subject individual or cloned from the subject individual. The cells may also have been engineered to exhibit a desired major histocompatability profile. The use of such cells overcomes the difficulties which are inherently encountered in the context of tissue and organ transplants. However, where it is not possible or feasible to isolate or generate autologous cells, it may be necessary to utilise allogeneic stem cells. “Allogeneic” stem cells are those which are isolated from the same species as the subject being treated but which exhibit a different MHC profile. Although the use of such cells in the context of therapeutics would likely necessitate the use of immunosuppression treatment, this problem can nevertheless be minimised by use of cells which exhibit an MHC profile exhibiting similarity to that of the subject being treated, such as a cellular population which has been isolated/generated from a relative such as a sibling, parent or child. The present invention should also be understood to extend to xenogeneic transplantation. That is, the cells which are introduced into a patient are isolated from a species other than the species of the subject being treated. It should be understood that these principles also apply to the situation where a population of cells is administered to a patient for the purpose of modulating transition in vivo.

Without limiting the present invention to any one theory or mode of action, even the partial amelioration or treatment of a condition can be beneficial, or desirable to a patient. Accordingly, reference to an “effective number” means that number of cells necessary to at least partly attain the desired effect, or to delay the onset of, inhibit the progression of, or halt altogether the onset or progression of the particular condition being treated. Such amounts will depend, of course, on the particular conditions being treated, the severity of the condition and individual patient parameters including age, physical condition, size, weight, physiological status, concurrent treatment, medical history and parameters related to the disorder in issue. One skilled in the art would be able to determine the number of cells that would constitute an effective dose, and the optimal mode of administration thereof without undue experimentation. These factors are well known to those of ordinary skill in the art and can be addressed with no more than routine experimentation. It is preferred generally that a maximal cell number be used, that is, the highest safe number according to sound medical judgement. It will be understood by those of ordinary skill in the art, however, that a lower cell number may be administered for medical reasons, psychological reasons or for any other reasons.

Cells which are administered to the patient can be administered as single or multiple doses by any suitable route. Preferably, and where possible, a single administration is utilised. Administration via injection can be directed to various regions of a tissue or organ, depending on the type of repair required.

It would be appreciated that in accordance with these aspects of the present invention, the cells which are administered to the patient may take any suitable form, such as being in a cell suspension or taking the form of a tissue graft. In terms of generating a single cell suspension, the culture protocol may be designed such that it favours the maintenance of a cell suspension. Alternatively, if cell aggregates or tissues form under the influence of the culture conditions which are utilised, these may be dispersed into a cell suspension. In terms of utilising a cell suspension, it may also be desirable to select out specific subpopulations of cells for administration to a patient, such as terminally differentiated cells. To the extent that it is desired that a tissue is transplanted into a patient, this will usually require surgical implantation (as opposed to administration via a needle or catheter). Alternatively, a portion, only, of this tissue could be transplanted. In another example, engineered tissues can be generated via standard tissue engineering techniques, for example by seeding a tissue engineering scaffold having the designed form with the cells and tissues of the present invention and culturing the seeded scaffold under conditions enabling colonization of the scaffold by the seeded cells and tissues, thereby enabling the generation of the formed tissue. The formed tissue is then administered to the recipient, for example using standard surgical implantation techniques. Suitable scaffolds may be generated, for example, using biocompatible, biodegradable polymer fibers or foams, comprising extracellular matrix components, such as laminins, collagen, fibronectin, etc. Detailed guidelines for generating or obtaining suitable scaffolds, culturing such scaffolds and therapeutically implanting such scaffolds are available in the literature (for example, refer to Kim S. S, and Vacanti J. P., 1999. Semin Pediatr Surg. 8:119, U.S. Pat. No. 6,387,369 to Osiris, Therapeutics, Inc.; U.S. Pat. App. No. US20020094573A1 to Bell E.).

In accordance with the method of the present invention, other proteinaceous or non-proteinaceous molecules may be co-administered either with the introduction of the subject cells or agent or prior or subsequently thereto. By “co-administered” is meant simultaneous administration in the same formulation or in different formulations via the same or different routes or sequential administration via the same or different routes. By “sequential” administration is meant a time difference of from seconds, minutes, hours or days between the introduction of these cells or agents and the administration of the proteinaceous or non-proteinaceous molecules or the onset of the functional activity of these cells or agents and the administration of the proteinaceous or non-proteinaceous molecule. Examples of circumstances in which such co-administration may be required include, but are not limited to:
(i) When administering non-syngeneic cells or tissues to a subject, there usually occurs immune rejection of such cells or tissues by the subject. In this situation it would be necessary to also treat the patient with an immunosuppressive regimen, preferably commencing prior to such administration, so as to minimise such rejection. Immunosuppressive protocols for inhibiting allogeneic graft rejection, for example via administration of cyclosporin A, immunosuppressive antibodies, and the like are widespread and standard practice.
(ii) Depending on the nature of the condition being treated, it may be necessary to maintain the patient on a course of medication to alleviate the symptoms of the condition until such time as the transplanted cells become integrated and fully functional. Alternatively, at the time that the condition is treated, it may be necessary to commence the long term use of medication to prevent re-occurrence of the damage. For example, where the condition is cancer an ongoing chemotherapy or radiation therapy may be performed.

It should also be understood that the method of the present invention can either be performed in isolation to treat the condition in issue or it can be performed together with one or more additional techniques designed to facilitate or augment the subject treatment. These additional techniques may take the form of the co-administration of other proteinaceous or non-proteinaceous molecules, as detailed hereinbefore.

The agents of the present invention are conveniently formulated in pharmaceutical compositions by adding an effective amount of a compound to a suitable pharmaceutically acceptable diluent or carrier. Use of the compounds and methods of the invention may also be useful prophylactically.

Accordingly, reference to a “agent” (including an antagomer or agomer), “chemical agent”, “compound”, “pharmacologically active agent”, “medicament”, “active” and “drug” includes combinations of two or more active agents. A “combination” also includes multi-part such as a two-part composition where the agents are provided separately and given or dispensed separately or admixed together prior to dispensation. For example, a multi-part pharmaceutical pack may have two or more agents separately maintained. Hence, this aspect of the present invention includes combination therapy. Combination therapy includes the co-administration of an agent which modulates miRNA levels and one or more cytokines which are involved in the biological process of EMT and/or one or more chemotherapeutic agents.

Examples of chemotherapeutic agents include but are not limited to cancer chemotherapeutic drugs such as daunorubicin, daunomycin, dactinomycin, doxorubicin, epirubicin, idarubicin, esorubicin, bleomycin, mafosfamide, ifosfamide, cytosine ara-binoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine, 5-azacytidine, hydroxyurea, deoxyco-formycin, 4-hydroxyperoxycyclophosphoramide, 5-fluorouracil (5-FU), 5-fluorodeoxyuridine (5-FUdR), methotrexate (MTX), colchicine, taxol, vincristine, vinblastine, etoposide (VP-16), trimetrexate, irinotecan, topotecan, gemcitabine, teni-poside, cisplatin and diethylstilbestrol (DES).

Accordingly, in yet another embodiment of the present invention there is provided the use an agent capable of elevating or reducing miRNA levels in the manufacture of a medicament to modulate EMT in a subject.

The pharmaceutical formulations of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, gel capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

Formulations of the present invention include liposomal formulations. As used in the present invention, the term “liposome” means a vesicle composed of amphiphilic lipids arranged in a spherical bilayer or bilayers. Liposomes are unilamellar or multilamellar vesicles which have a membrane formed from a lipophilic material and an aqueous interior that contains the composition to be delivered. Cationic liposomes are positively charged liposomes which are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860, which is incorporated herein in its entirety.

The formulation of therapeutic compositions and their subsequent administration (dosing) is within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models. In general, dosage is from 0.01 μg to 100 g per kg of body weight, and may be given once or more daily, weekly, monthly or yearly. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.01 μg to 100 g per kg of body weight, once or more daily, to once every 20 years.

In a related aspect of the present invention, the subject undergoing treatment or prophylaxis may be any human or animal in need of therapeutic or prophylactic treatment. In this regard, reference herein to “treatment” and “prophylaxis” is to be considered in its broadest context. The term “treatment” does not necessarily imply that a mammal is treated until total recovery. Similarly, “prophylaxis” does not necessarily mean that the subject will not eventually contract a disease condition. Accordingly, treatment and prophylaxis include amelioration of the symptoms of a particular condition or preventing or otherwise reducing the risk of developing a particular condition. The term “prophylaxis” may be considered as reducing the severity of the onset of a particular condition. “Treatment” may also reduce the severity of an existing condition.

Another aspect of the present invention is directed to the use of a population of cells treated in accordance with the method of the invention in the manufacture of a medicament for the treatment of a condition.

Yet another aspect of the present invention is directed to a population of cells treated in accordance with the method of the invention, or cells differentiated therefrom.

Yet another aspect of the present invention is directed to the use of an agent which either (i) elevates the functional level of an miRNA or family of miRNAs or functional fragment or derivative thereof or (ii) reduces the functional level of an miRNA or family of miRNAs or functional fragment or derivative thereof in epithelial or mesenchymal cells, which miRNAs are differentially expressed in either cell type in tissue undergoing EMT relative to epithelial tissue prior to, during or following EMT, in the manufacture of a medicament for the treatment of a condition wherein said agent modulates EMT wherein:
(i) upregulating the functional level of an miRNA which is downregulated in mesenchymal cells post EMT relative to epithelial cells, inhibits or down-regulates EMT;
(ii) downregulating the functional level of an miRNA which is downregulated in mesenchymal cells post EMT relative to epithelial cells induces or upregulates EMT;
(iii) upregulating the functional level of an miRNA which is upregulated in mesenchymal cells post EMT relative to epithelial cell induces or upregulates EMT;
(iv) downregulating the functional level of an miRNA which is upregulated in mesenchymal cells post EMT relative to epithelial cells inhibits or down-regulates EMT;
(v) upregulating the functional level of an miRNA which is downregulated in mesenchymal cells post EMT relative to epithelial cells induces or upregulates MET; and
(vi) downregulating the functional level of an miRNA which is upregulated in mesenchymal cells post EMT relative to epithelial cells induces or upregulates MET.

In one preferred embodiment there is provided the use of an agent which upregulates the functional level of one or more miRNAs or family of miRNAs or functional fragment or derivative thereof, wherein said miRNA is upregulated in epithelial cells compared to mesenchymal cells following EMT, in the manufacture of a medicament for the treatment of a condition wherein said agent downregulates EMT.

Preferably, said miRNAs are defined by SEQ ID NOs:1-11 or 19.

More preferably, said miRNAs are defined by SEQ ID NOs:1-5 or 19.

In another preferred embodiment, said miRNA is defined by SEQ ID NO 6.

In still another preferred embodiment there is provided the use of an agent which downregulates the functional level of one or more miRNAs or family of miRNAs or functional fragment or derivative thereof, wherein said miRNA is downregulated in epithelial cells compared to mesenchymal cells following EMT, in the manufacture of a medicament for the treatment of a condition, wherein said agent downregulates EMT.

Preferably, said miRNAs are defined by SEQ ID NOs:12-18.

In still another preferred embodiment there is provided the use of an agent which downregulates the functional level of one or more miRNAs or family of miRNAs or functional fragment or derivative thereof, wherein said miRNA is downregulated in mesenchymal cells compared to epithelial cells after EMT, in the manufacture of a medicament for the treatment of a condition, wherein said agent upregulates EMT. Preferably, said miRNAs are defined by SEQ ID NOs:1-11 or 19.

More preferably, said miRNAs are defined by SEQ ID NOs:1-5 or 19.

In another preferred embodiment, said miRNA is defined by SEQ ID NO 6.

In yet another preferred embodiment there is provided the use of an agent which upregulates the functional level of one or more miRNAs or family of miRNAs or functional fragment or derivative thereof, wherein said miRNA is upregulated in mesenchymal cells compared to epithelial cells following EMT, in the manufacture of a medicament for the treatment of a condition, wherein said agent upregulates EMT.

Preferably, said miRNAs are defined by SEQ ID NOs:12-18.

In still another preferred embodiment there is provided the use of an agent which upregulates the functional level of one or more miRNAs or family of miRNAs or functional fragment or derivative thereof, wherein said miRNA is downregulated in mesenchymal cells compared to epithelial cells after EMT, in the manufacture of a medicament for the treatment of a condition, wherein said agent upregulates EMT.

Preferably, said miRNAs are defined by SEQ ID NOs:1-11 or 19.

More preferably, said miRNAs are defined by SEQ ID NOs:1-5 or 19.

In another preferred embodiment, said miRNA is defined by SEQ ID NO 6.

In yet another preferred embodiment there is provided the use of an agent which downregulates the functional level of one or more miRNAs or family of miRNAs, or a functional analog thereof wherein said miRNA is upregulated in mesenchymal cells compared to epithelial cells following EMT, in the manufacture of a medicament for the treatment of a condition, wherein said agent upregulates EMT.

Preferably, said miRNAs are defined by SEQ ID NOs:12-18.

The methods of the present invention are also useful in diagnostic protocols for monitoring or predicting EMT or MET based on determining changing levels of specific miRNAs or families of miRNAs.

Accordingly, another embodiment of the present invention provides a method for detecting EMT or MET or for determining the likelihood of EMT or MET development or monitoring the state of EMT or MET in a subject, said method comprising detecting one or more miRNAs which are either elevated or reduced in tissue undergoing EMT or MET wherein the presence, level or profile of expression of said miRNAs is indicative of EMT or MET or its progression.

A range of DNA or modified RNA arrays and other genetic analyses may be used to screen for levels of defined miRNAs. Profiles of levels, presence and/or absence of miRNAs or families of miRNAs provide a signature of EMT or MET or a propensity for the development of EMT or MET. Such a signature may be important, for example, in monitoring for potential metastasis of a cancer, the ability for a wound to heal or for an organ or tissue to become fibrotic.

For use in kits and diagnostics, the agents of the present invention, either alone or in combination with other agents or therapeutics, can be used as tools in differential and/or combinatorial analyses to elucidate expression patterns of a portion or the entire complement of genes expressed within cells and tissues.

As one non-limiting example, expression patterns within cells or tissues treated with one or more agents are compared to control cells or tissues not treated with the agents and the patterns produced are analyzed for differential levels of gene expression as they pertain to the EMT process or its physiological effect.

Examples of methods of gene expression analysis known in the art include DNA arrays or microarrays (Brazma and Vilo, FEBS Lett. 480: 17-24, 2000; Celis et al., FEBS Lett. 480: 2-16, 2000), SAGE [serial analysis of gene expression] (Madden et al., Drug Discov. Today 5: 415-425, 2000), READS (restriction enzyme amplification of digested cDNAs) (Prashar and Weissman, Methods Enzymol. 303: 258-272, 1999), TOGA (total gene expression analysis) (Sutcliffe et al., Proc. Natl. Acad. Sci. USA 97: 1976-1981, 2000), protein arrays and proteomics (Celis et al. 2000, supra; Jungblut et al., Electrophoresis 20: 2100-2110, 1999), expressed sequence tag (EST) sequencing (Celis et al., 2000, supra; Larsson et al., J. Biotechnol. 80: 143-157, 2000), subtractive RNA fingerprinting (SuRF) (Fuchs et al., Anal. Biochem. 286: 91-98, 2000; Larson et al., Cytometry 41: 203-208, 2000), subtractive cloning, differential display (DD) (Jurecic and Belmont, Curr. Opin. Microbiol. 3: 316-321, 2000), comparative genomic hybridization (Carulli et al., J. Cell Biochem. Suppl. 31: 286-296, 1998), FISH (fluorescent in situ hybridization) techniques (Going and Gusterson, Eur. J. Cancer, 35: 1895-1904, 1999) and mass spectrometry methods (To, Comb. Chem. High Throughput Screen, 3: 235-241, 2000).

Combination therapy comprising targeting for reduction or elevation of certain miRNAs together with cytokine or other proteomic therapy (inhibitory and/or replacement) also forms part of the present invention.

The present invention also extends to animal models comprising genetically modified cells or cells derived from genetically modified cells which express an miRNA or family of miRNAs or which are no longer capable of producing one or more miRNAs or which carry genetic material modified to express or not express an miRNA recognition motif. Such animal models are useful for screening for therapeutic agents and for determining the effects of miRNAs on various biological processes. In addition, cells may be engineered to express one or more miRNAs or genes engineered to express an introduced miRNA recognition motif or to delete an miRNA recognition sequence. Such genes become either sensitive or insensitive to miRNA repression.

Preferred genetically modified cells are those from mice, rats, pugs, goats and non-human primates.

In one embodiment, the genetically modified cell is substantially incapable of generating an miRNA selected from SEQ ID NOs:1 through 18.

In another embodiment, the genetically modified cell or a parent cell carries exogenously introduced expressible DNA which encodes an miRNA selected from SEQ ID NOs:1 through 18.

In still another embodiment, the genetically modified cell or a parent cell comprises exogenously introduced DNA which either comprises an inserted miRNA recognition motif or a deleted miRNA recognition motif for an miRNA selected from SEQ ID NOs:1 through 18.

The present invention is further described by the following non-limiting examples.

### Example 1

### Identification of miRNAs

MicroRNA microarrays were used to survey changes in microRNA levels in cells when they underwent EMT. Several microRNAs were identified that were strongly reduced in expression after the EMT. These microRNAs, when expressed, may help to maintain the epithelial phenotype by suppressing genes that would promote the EMT.

Some other microRNAs were increased in the cells that had undergone EMT. These may help promote the EMT or help maintain cells in a mesenchymal state.

Of the microRNAs that were reduced after the EMT, the most prominent were microRNA 205, and a family of highly related microRNAs:

| | | |
|---|---|---|
| hsa-miR-200a | UAACACUGUCUGGUAACGAUGU- | SEQ ID NO: 1 |
| hsa-miR-429 | UAAUACUGUCUGGUAAAACCGU- | SEQ ID NO: 2 |
| hsa-miR-200b | UAAUACUGCCUGGUAAUGAUGAC | SEQ ID NO: 3 |
| hsa-miR-141 | UAACACUGUCUGGUAAAGAUGG- | SEQ ID NO: 4 |
| hsa-miR-200c | UAAUACUGCCGGGUAAUGAUGG | SEQ ID NO: 5 |

Pooled date for 3 hybridizations are provided in Table 2.

TABLE 2

| | | | | | | | |
|---|---|---|---|---|---|---|---|
| SEQ ID | | log2 (fold | average intensity | | | | |
| NO: | Name | change) | on slide | t statistic | P.Value | B | Sequence |

| | | | | | | | |
|---|---|---|---|---|---|---|---|
| 5 | hsa_miR_200c | −1.36641815 | 9.312521859 | −12.40424735 | 1.60E-010 | 19.94 | uaauacugccggguaaugaugg |
| 3 | hsa_miR_200b | −1.215129539 | 9.430999112 | −11.09811325 | 1.17E-009 | 17.32 | uaauacugccugguaaugaugac |
| 6 | hsa_miR_205 | −2.400193323 | 9.488531311 | −10.68872912 | 1.88E-009 | 16.46 | uccuucauuccaccggagucug |
| 7 | hsa_miR 23a | −0.455265795 | 11.67195674 | −8.50270494 | 2.23E-007 | 11.46 | aucacauugccagggauuucc |
| 4 | hsa miR 141 | −0.529958778 | 8.930550812 | −7.01421408 | 8.02E-006 | 7.686 | uaacacugucugguaaagaugg |
| 1 | hsa_miR_200a | −0.413471789 | 8.739232314 | −6.889070151 | 9.31E-006 | 7.357 | uaacacugucugguaacgaugu |
| 8 | hsa_miR_27a | −0.323660816 | 10.99298282 | −6.20981953 | 4.97E-005 | 5.544 | uucacaguggcuaaguuccgc |
| 12 | hsa_miR_181b | 0.396575297 | 9.39329391 | 5.433901109 | 0.0003675 | 3.434 | aacauucauugcugucgguggg |
| 13 | hsa_miR_181a | 0.431075047 | 9.399554131 | 5.120862355 | 0.0007649 | 2.578 | aacauucaacgcugucggugagu |
| 14 | hsa_miR_29a | 0.364337276 | 9.796661176 | 5.075727472 | 0.0007649 | 2.455 | uagcaccaucugaaaucgguu |
| 2 | mmu_miR_429 | −0.469222278 | 8.636417274 | −5.054901649 | 0.0007649 | 2.398 | uaauacugucugguaaaaccgu |
| 9 | hsa_miR_31 | −0.537419694 | 11.19865416 | −4.608421468 | 0.0024187 | 1.183 | ggcaagaugcuggcauagcug |
| 15 | hsa_miR_155 | 0.538942554 | 9.869983654 | 4.491146229 | 0.0029809 | 0.866 | uuaaugcuaaucgugauagggg |
| 10 | hsa_miR_106a | −0.277100865 | 9.775702043 | −4.476998751 | 0.0029809 | 0.828 | aaaagugcuuacagugcagguagc |
| 16 | hsa_miR_27b | 0.329860576 | 9.863776879 | 4.437971041 | 0.0030984 | 0.723 | uucacaguggcuaaguucugc |
| 17 | hsa miR_422b | 0.3729078 | 9.581531792 | 4.201111505 | 0.0055683 | 0.089 | cuggacuuggagucagaaggcc |
| 18 | hsa_miR_22 | 0.378745602 | 9.469746926 | 4.120229742 | 0.0065369 | −0.126 | aagcugccaguugaagaacugu |
| 11 | hsa miR_194 | −0.29045079 | 9.011039095 | −3.91135788 | 0.0108849 | −0.675 | uguaacagcaacuccaugugga |

Of the micro RNAs detailed in Table 2, those which were reduced after EMT correspond to SEQ ID NOs:1-11, while those which were increased correspond to SEQ ID NOs:12-18.

### Example 2

### Predicting microRNA targets

MicroRNAs function as the specificity component of a protein-RNA complex that binds in a sequence-specific manner to the 3′UTR of target mRNAs, resulting in inhibition of protein translation from the mRNA, and promoting degradation of the target mRNA. In metazoa, nucleotides 2 to 8 of the microRNA are the principal determinants of specificity, while the remainder of the microRNA sequence needs only to loosely pair with the target mRNA. The strict requirement for base pairing of nucleotides 2-7 (and preference for pairing by nucleotide 8) allows prediction of potential mRNA targets for each particular microRNA. MicroRNAs have been highly conserved during evolution; many are absolutely conserved across vertebrates, and even in insects and C. elegans. The target sites within mRNAs are usually conserved too, maintained by the selective pressure to retain the functionality of the microRNA-mRNA interaction. This is in contrast to mRNA 3′UTR sequences in general, which tend to be highly divergent across species. The unusually high conservation of the target site sequence within a target mRNA allows sequence-based prediction of mRNA targets with quite high degree of reliability. Experimental testing using reporter genes verifies individual predictions that take into account evolutionary conservation of the sites in the majority of cases. In one report, 11 predicted targets out of 15 were supported experimentally using a reporter assay in HeLa cells. However, although many genes are predicted to be targeted by one or more microRNAs, there are few validated interactions reported to date, and for most microRNAs (including the miR-200 family and miR-205) no targets have been determined experimentally.

Overexpression of Pez in Epithelial MDCK Cells Leads to an EMT with Loss of E-Cadherin Expression and Induction of ZEB1 Expression.

An in vitro model of EMT has been developed, in which MDCK canine epithelial cells are transfected to stably overexpress the protein tyrosine phosphatase, Pez. The Pez transfectants undergo a transformation to a fibroblastoid morphology with increased motility and invasiveness, reduced E-cadherin expression and increased expression of the mesenchymal markers, Snail, ZEB1, ZEB2 and fibronectin (FIG. 2).

### ZEB2

Zinc finger E-box-binding protein 2 (ZEB2), also known as ZFHX1B participates in EMT during early neuronal development, where it is required for delamination and migration of cranial neural crest cells. During epithelial dedifferentiation, ZEB2 coordinately represses the transcription of genes coding for junctional proteins, contributing to the dedifferentiated state. E-cadherin plays a crucial role in EMT and its loss in cancer is associated with de-differentiation, invasion and metastasis. ZEB2 represses E-cadherin expression by binding to E-box motifs in the E-cadherin promoter and ectopic expression of ZEB2 is sufficient to cause EMT in vitro. In tumours, loss of E-cadherin expression tends to correlate with upregulation of ZEB2, depending on the type of tumour. In some tumours, loss of E-cadherin is associated with DNA hypermethylation and chromatin rearrangements, or with the expression of other E-box-binding repressors such as Snail and ZEB1.

### ZEB1

Zinc finger E-box-binding protein 1 (ZEB1), also known as δEF1 (gene name: TCF8) represses E-cadherin expression by binding to E-box motifs in the E-cadherin promoter. Forced expression of ZEB1 in epithelial cells is sufficient to downregulate E-cadherin and induce EMT. Among the known transcriptional repressors of E-cadherin, ZEB1 was found to be uniquely correlated with E-cadherin loss in lung cancer cell lines, and this loss of E-cadherin could be reversed by RNAi-mediated knockdown of ZEB1. The same has been found in breast cancer cell lines.

Accompanying the reduction in E-cadherin mRNA in the Pez-MDCK cells was an increase in the expression of Snail (FIG. 2), a transcription factor pivotal in downregulating E-cadherin. In addition, ZEB1, another repressor of E-cadherin transcription commonly induced in EMTs was also induced by Pez (FIG. 2a). Together morphological and biochemical data indicated that the Pez-MDCK cells had undergone EMT.

### Pez Expression is Developmentally Regulated

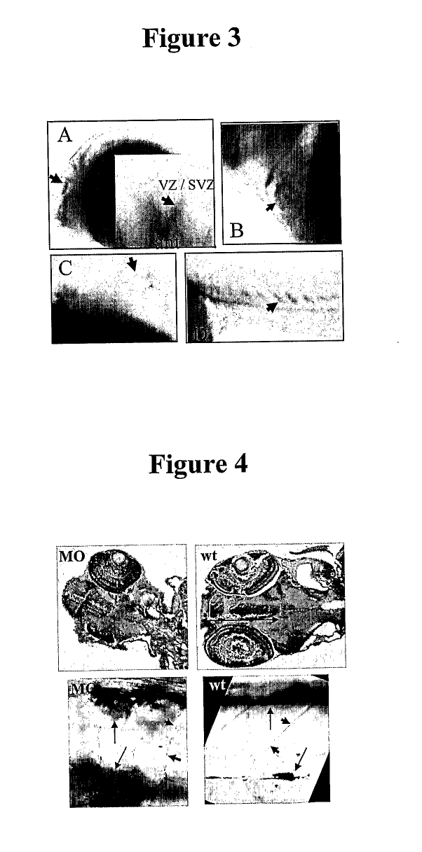

The primary role of EMT is for generation of new tissues and organs during embryonal development. Using the zebrafish as a model organism it was found by whole mount in situ hybridisation that the expression of Pez is temporally and spatially regulated during development. It is transiently expressed in a number of developing organs, with peak expression in individual organs occurring at different times during development (for examples see Table 3 & FIG. 3). Furthermore, in some organs Pez expression was confined to specific regions, e.g. in the ventricular/subventricular zone (VZ/SVZ) in the brain at 24 hpf, and in specific cells of the growing tip of the pectoral fin bud at 42-48 hpf (FIG. 3). These observations are consistent with Pez playing a role in regulating EMT, a process expected to be transiently induced during specific phases in the development of new organs.

### Pez Morphants Exhibit Defects in Tissue/Organ Development

Pez zebrafish morphants, generated by injection of antisense morpholino oligonucleotides (MOs) (designed to inhibit translation of Pez) into 1-to-4-cell stage embryos, had developmental defects (summarised in Table 4 & FIG. 4) in all the organs where Pez expression was detected. Three different antisense MOs with a range of GC contents targeted to different sequences to inhibit translation consistently gave rise to the same set of defects, not seen with control MO-injected embryos that are indistinguishable from uninjected controls. In addition to its role in EMT, Snail also regulates many EMT-independent processes such as cell migration, cell death, cell survival and left-right asymmetry.

### Pez Overexpression Induces Hey1 Expression

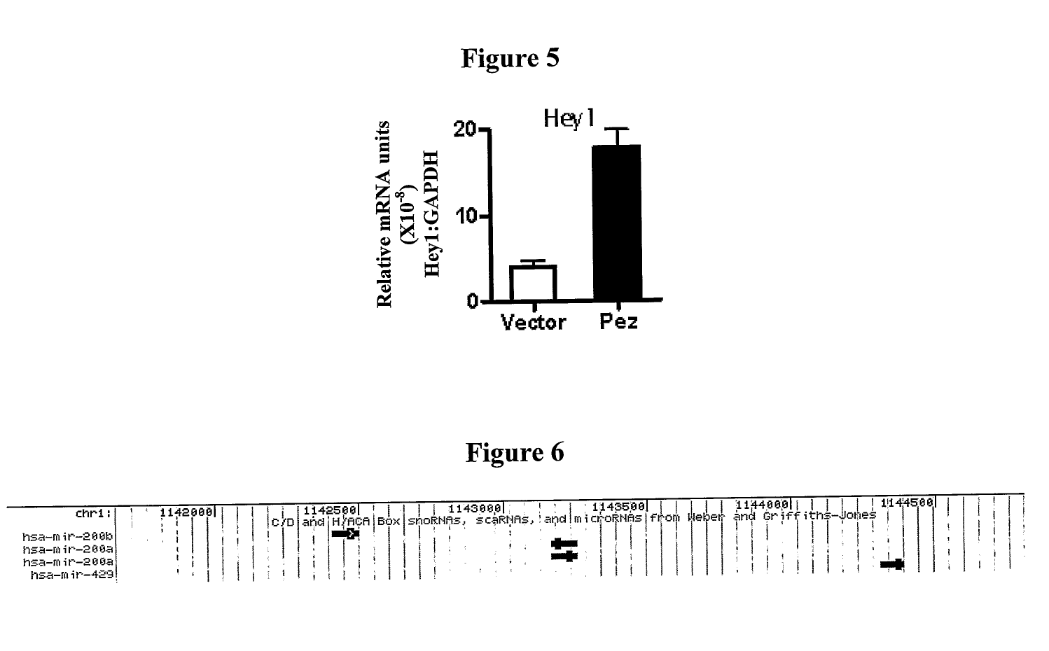

The Notch signalling pathway has been implicated in regulating EMT by upregulating Snail expression in vitro and in vivo, particularly with respect to cardiac valve formation. The Notch pathway is also implicated in development of many tissues and organs including vascular, melanocyte, central nervous system, limb and somites. Because many of the defects observed in the Pez morphants are similar to deficiencies in Notch signalling, the expression of Hey1, a Snail-independent downstream target of Notch was investigated, and found that Hey1 is induced in the Pez-transfected cells (FIG. 5).

### Pez Downregulates miR 205, 200a and 200b Expression

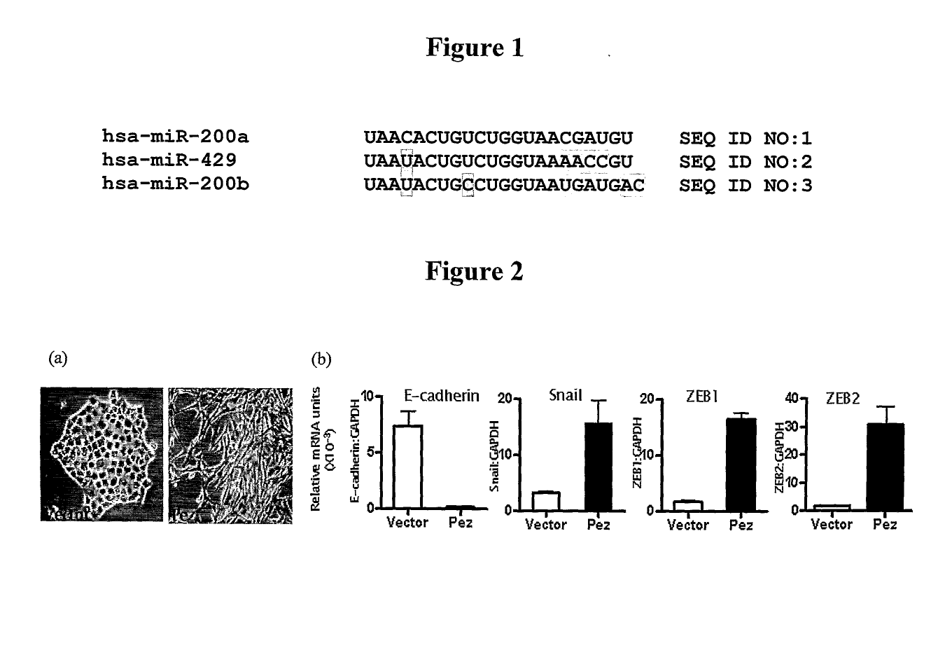

MiR-200a, miR-200b and miR-429 are closely clustered on human chromosome 1 and are likely to be expressed from a common precursor transcript (FIG. 1). MiR-200a and miR-200b especially, and to a lesser extent miR-429, were all found to be highly expressed in MDCK cells and strongly downregulated in MDCK-Pez, consistent with the notion they are expressed from a common transcript. MiR-205 is unrelated to other microRNAs and is not clustered with other microRNAs. In a study of microRNAs expressed in embryonic mouse skin, all of these microRNAs were found to be abundant in the epidermis, but not in hair follicles.

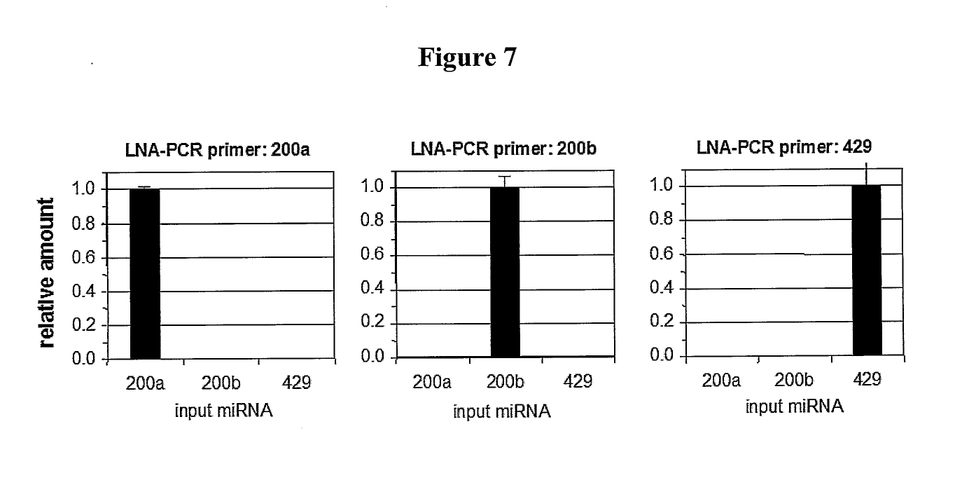

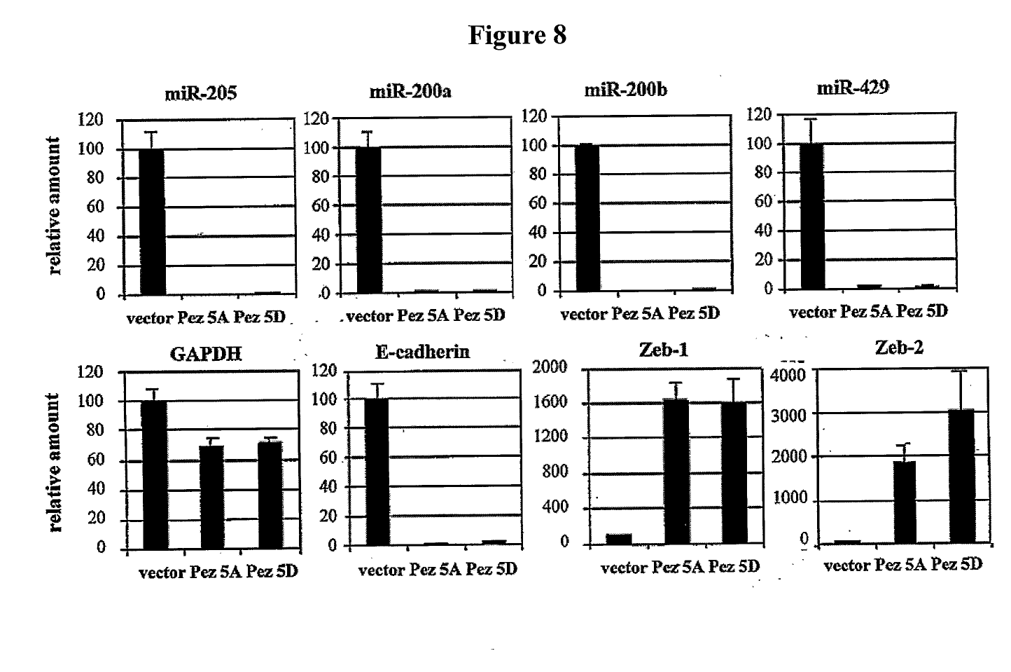

Highly specific quantitative real time PCR assays were developed for MiR-200a, miR-200b and miR-429 using locked nucleic acid (LNA) oligonucleotide primers (LNA-PCR) (FIG. 7). The LNA-PCR assays verified that miR-205 and the members of the miR-200a cluster are highly regulated (FIG. 8).

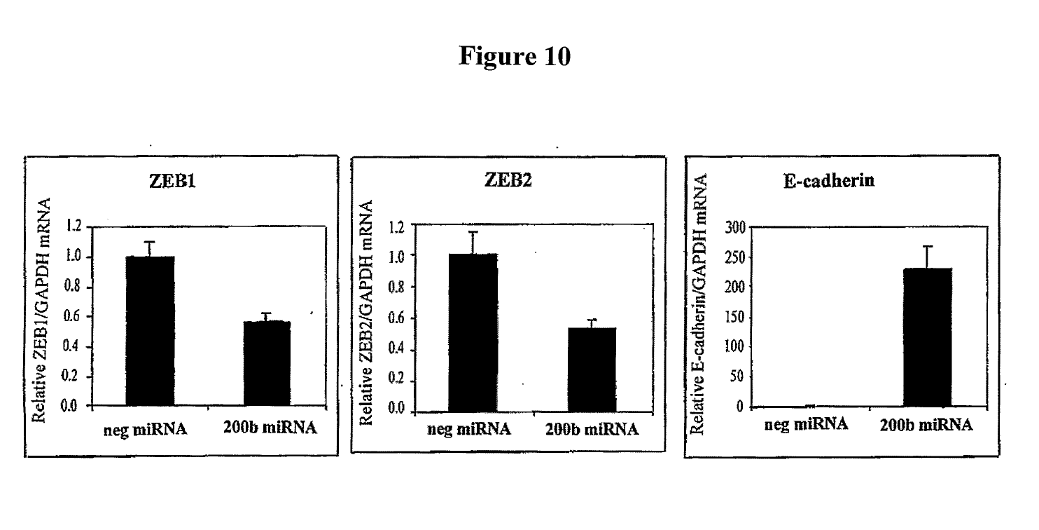

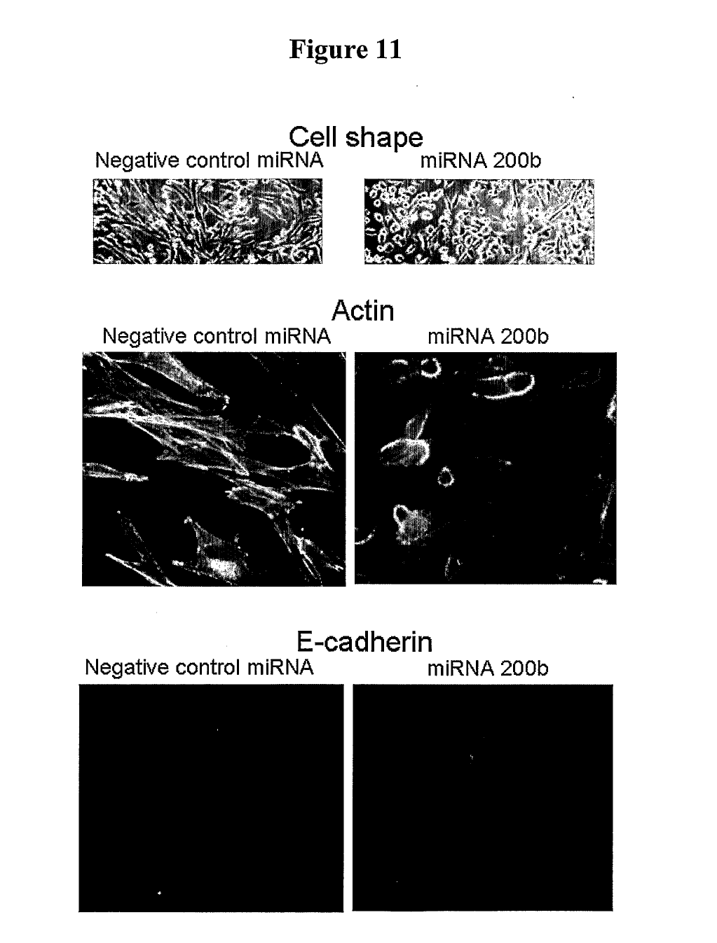

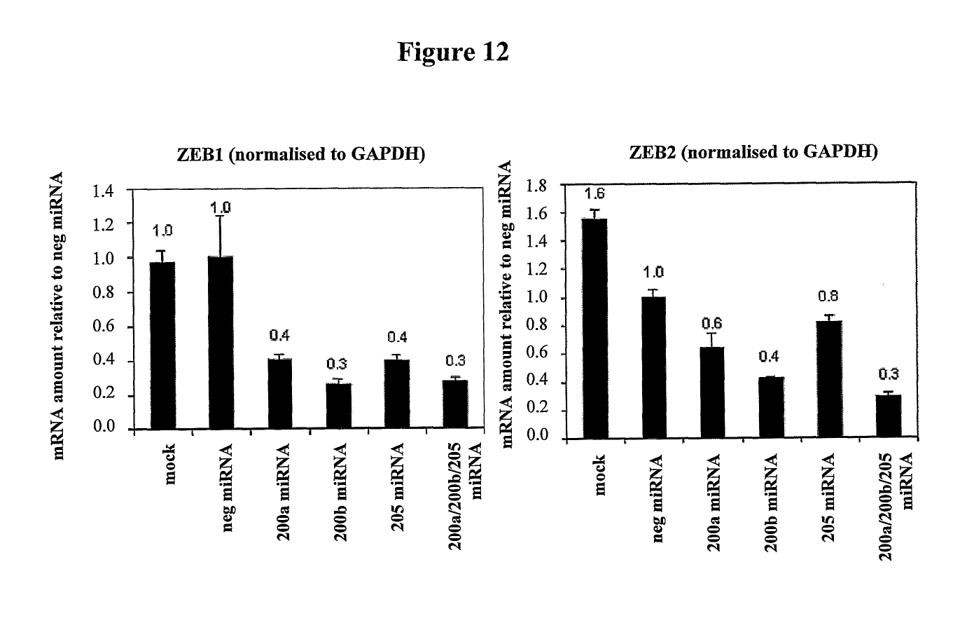

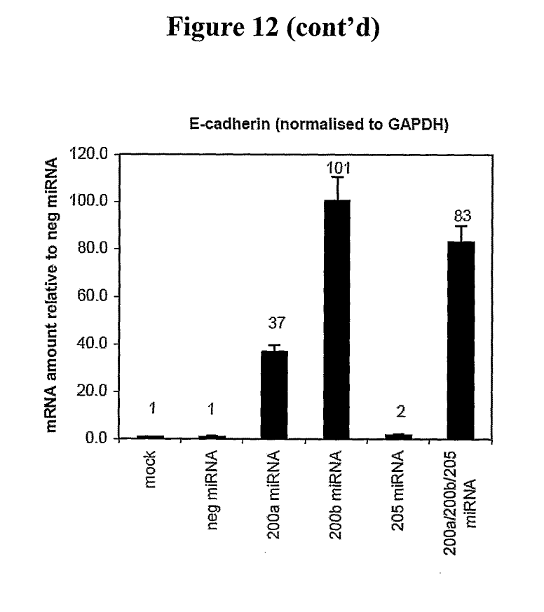

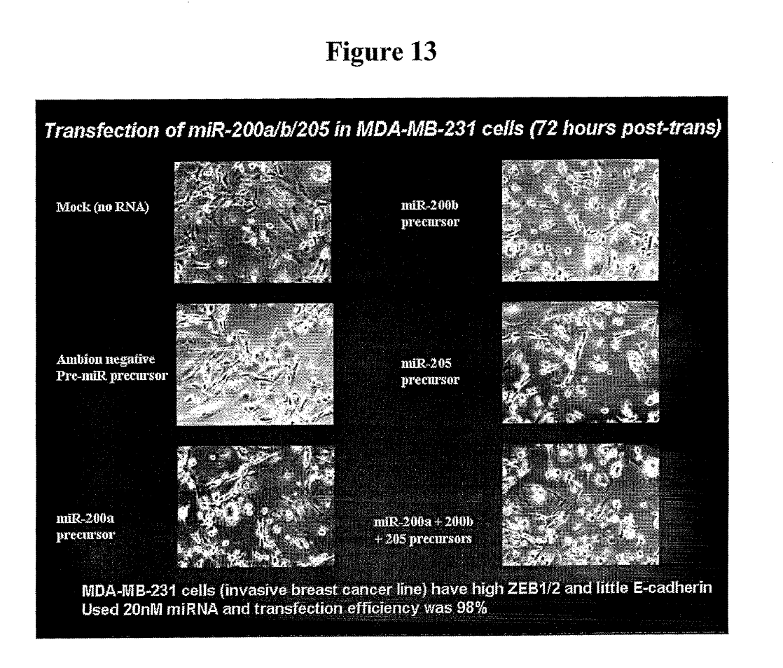

Overexpression of miR-200b in Pez-MDCK cells which exhibit a mesenchymal phenotype, characterised by high ZEBs and low E-cadherin and low to negligible miR-200b results in downregulation of ZEB1 and ZEB2 and upregulation of E-cadherin (FIG. 10). In addition overexpression of miR-200b in Pez-MDCK cells causes reversion to the epithelial phenotype, including a change in shape from fibroblast-like cells to epithelial-like cells. Morphological change is accompanied by reorganisation of actin filaments from stress fibres to cortical actin surrounding the cells and increased E-cadherin and relocalisation to the cell-cell junctions (FIG. 11). These results were also obtained when miRNAs-200a, -200b and ±205 were transfected into MDA-MB-231 human breast cancer cells (FIGS. 12 and 13).

### Screening for Inducers of Pez Expression

Cell lines (MDCK and NMuMG) are treated with known inducers of EMT (TGFβ, FGF, Notch, Wnt, EGF, PDGF, HGF) and Pez expression analysed by qRT-PCR at various times (1-5 days) after induction (both canine and mouse Pez sequences are available). Whether the treatments induced EMT is assessed by examining Snail expression (by qRT-PCR) as an early marker of EMT, induction of ZEB1 (by qRT-PCR) and fibronectin (by WB) and loss of E-cadherin expression (by qRT-PCR) as later markers of EMT. With TGFβ, FGF, EGF, PDGF and HGF, the growth factors (from commercial sources) are added exogenously to the culture medium. To determine whether Pez expression is downstream of Notch intracellular signalling, the Notch 1 intracellular domain (NICD) is transfected into cell lines and increased Hey1 expression (measured by qRT-PCR) used as an indicator of upregulated Notch signalling. Purified Wnt proteins are generally difficult to obtain, therefore canonical Wnt signalling is simulated by transfection with a mutant of β-catenin (4 S/Ts in GSK3β site mutated to A²¹, gift from P McCrea, Texas) that escapes proteosomal degradation and accumulates in the nucleus. In addition, canonical Wnt signalling is mimicked by treating cells with LiCl to inhibit GSK3β activity. Co-transfection with a TOPFlash reporter is used to indicate upregulation of β-catenin-dependent transcription. The T-Rex (Invitrogen) tet-inducible expression system for expressing NICD and β-catenin in MDCK cells that stably express the tet repressor is used; stable pools of Notch and β-catenin cells obtained following selection are used. Generally ˜50% transfection efficiency in NMuMG cells is obtained enabling the performance of these studies in transiently transfected cells with constitutive expression vectors; alternatively tet-inducible cell lines are generated as in the MDCK cells.

In Vitro Verification that Pez is an Essential Downstream Mediator of EMT Signalling Pathway(s)

Whether Pez is an essential mediator of the EMT-inducing signalling pathway is assessed by inhibiting its expression with siRNAs during induction. The t_{1/2} of the Pez protein is short (˜2 h, unpublished observation) and routinely good (˜70%) knock-down of human Pez protein expression with siRNAs is obtained. The efficacy of the siRNAs is assessed by Western Blot (WB) for Pez using an Ab that recognises mammalian Pez. Both the mouse (Genbank) and canine (NCBI canine WGS database) Pez sequences are available for designing siRNAs. Following transfection of siRNAs, cells are treated with the EMT-inducing factor and at various times after, Snail, ZEB1, fibronectin and E-cadherin expression assayed.

Verification that Pez is an In Vivo Mediator of EMT Signalling Pathways

The in vivo role of pathways found to regulate Pez expression in vitro is corroborated by knocking-down crucial mediators of the signalling pathway(s) in zebrafish and analysing (i) by whole mount in situ hybridisation whether Pez expression is decreased/absent in any organ(s) where Pez expression has been previously mapped, and (ii) whether the resulting morphants phenocopy the Pez morphants.

Correlation of Pez Induction with Microrna Downregulation in EMT Signalling.

Measurement of the levels of miR-205, miR-200a and miR-200b by LNA-PCR, is determined to see if they reciprocally correlate with Pez expression.

### The Kinetics of Microrna Downregulation in Response to Fez.

To investigate whether microRNA shut off is proximal to Pez expression, whether microRNA downregulation closely follows Pez expression, or alternatively, occurs only after other factors such as Snail or ZEB1 have been induced is determined by examining Pez expression in tet-ind Pez-MDCK cell lines. Pez expression is induced and RNA isolated at various times subsequently, ranging from hours to days. Pez, Snail and ZEB1 mRNA is measured by qRT-PCR and miR-205, miR-200a and miR-200b by LNA-PCR. To check whether Pez influences the degradation rate of the mature microRNAs, their half lives before and after Pez induction, is measured by LNA-PCR of RNA from actinomycin D time courses.

Expression of miR-200a, -200b and -205 to Expression of Pez in Zebrafish Embryos Compared.

The timing and location of Pez expression during zebrafish development by whole mount in situ hybridisation has been mapped (summarised in Table 3). To investigate the timing and location of expression of the three microRNAs by situ hybridisation using LNA probes at the corresponding times these are mapped, and compared to the Pez expression pattern. Mapping of Pez expression covered up to 48 hours post-fertilisation. MiR-205 is expressed from 12 hpf, while miR-200a and miR-200b are expressed by 20 hpf.

Verification that Pez Overexpression Activates Notch Signalling

(i) nuclear translocation of Notch intracellular domain (NICD). The critical step in activation of Notch signalling is the cleavage, release, and translocation of the NICD into the nucleus where it binds to RBPJK/CSL and acts as a transcription factor. Therefore, if nuclear translocation of NICD has occurred in Pez-MDCK clones compared to Vector-MDCK clones is determined by immunofluorescence (IF) using an Ab that detects only the cleaved NICD (Notch1 Val1744 Ab, Cell Signalling Technologies). WB analysis of fractionated nuclear and cytoplasmic cell lysates is used to confirm IF data. The analysis is also carried out in tet-ind Pez-MDCK cell lines to determine the time course of induction of Notch signalling following induction of Pez. A comparison of the time course of Notch activation with that of Pez expression indicates whether Notch activation is an early event following induction of Pez expression.

(ii) RBPJκ/NICD-luc reporter analysis.

Whether Pez induces Notch activation can also be assessed using a RBPJκ-luc (luciferase) reporter construct. The construct consists of 4 copies of the RBPJκ binding site (CGTGGGAAA) interspersed by a 27 by spacer sequence upstream of the minimal SV40 promoter driving luciferase in the pGL3 vector (Promega). A negative control where each of the RBPJκ-binding sites is mutated (CTACGGAAA)²⁵ is also generated. The reporters are transfected into tet-ind Pez-MDCK cells for analysis with and without induction of Pez.

(iii) RBPJκ siRNA knock-down.

Whether induction of Snail by Pez is dependent on Notch signalling. This is carried out by transfecting RBPJκsiRNA into tet-ind Pez-MDCK cell lines prior to addition of doxycyclin to induce Pez expression. The efficacy and duration of RBPJκ knock-down is checked by WB with anti-RBPJκ (Santa Cruz Biotechnology) or by monitoring induction of Hey1 (by qRT-PCR) by Pez. The canine RBPJκmRNA sequence for designing siRNAs is available from Genbank (gb|DN904913.1; gb|CO687929.1).

Identification of Components of the Signalling Pathway from Pez to Snail

The regions in the Snail promoter responsive to Pez signalling are identified and used to identify the transcription factors responsible for induction of Snail by Pez.

(i) Identification of Pez-responsive elements on the Snail promoter.

Human Snail promoter-luciferase (Snail-luc) constructs (provided as collaboration by Prof. A. Garcia de Herreros, Spain) are used to confirm that Pez activates Snail transcription and to identify regions in the Snail promoter responsive to Pez signalling. Starting from full-length (−1558/+92) and minimal (−78/+59) promoter-luc constructs, various intermediate truncations of the promoter are used to define a minimum sequence required. These analyses are performed in tet-ind Pez-MDCK cell lines. (Alternatively, the Snail-luc constructs are transiently transfected into MDCK cells with co-transfection of Renilla luciferase plasmid to normalise transfection efficiencies).

(ii) Identification of Transcription Factors that Bind to Pez-Responsive Elements.

The sequence for known transcription factor consensus binding sites is examined. The involvement of a consensus site is confirmed by inactivating it by mutation and testing for loss of Pez-responsiveness in the luc-reporter assays. To confirm the identity of the transcription factor, electrophoretic mobility shift assays (EMSAs) are conducted with the responsive region as probe, comparing complexes formed with extracts from Pez-MDCK and vector-MDCK cells. The role of the transcription factor in Pez-induced Snail transcription is further verified by knocking-down its expression with siRNA in tet-ind Pez-MDCK cells and demonstrating a loss of Pez-induced Snail-luc reporter activity as a consequence. The Pez-response element is mapped by truncation of the Snail-luc reporter, preferably to within 25 bp, and confirm complex formation on the minimal sequence by EMSA confirmed.

f. Identification of Novel Pez Substrates or Interacting Proteins

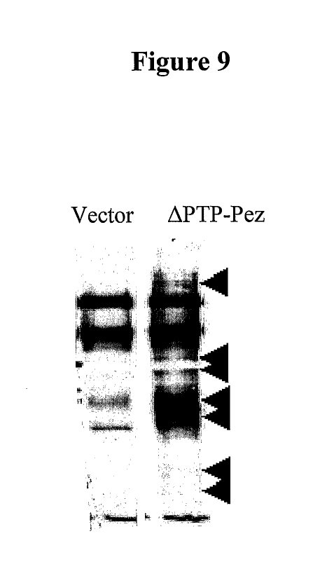

‘Substrate trapping’ is used to identify additional Pez substrates in epithelial cells. Transfection of MDCK or A431 epithelial cells with a dominant negative Pez mutant (ΔPTP-Pez) resulted in an increase in a number of tyrosine phosphorylated proteins specific to ΔPTP-Pez (FIG. 9). To identify novel Pez substrates, the ‘substrate trapping’ (ST) approach is used, this is the method of choice because the ST mutant cannot cleave the phosphate group on the substrate and so retains binding to its phosphorylated target that might otherwise interact too transiently to be ‘pulled-down’ or coIPed. The Pez ST mutant (D1079A) has been used successfully to identify β-catenin as a Pez substrate in endothelial cells. This same strategy is also used in this study except that MDCK cell lysates from cells pretreated with pervanadate (to inhibit intracellular tyrosine phosphatases and hence allow tyrosine phosphorylated proteins to accumulate) are used as a source of tyrosine phosphorylated substrates. Proteins binding to and hence ‘pulled-down’ by the ST mutant are resolved by SDS-PAGE, bands excised, trypsinised and identified by mass spectroscopy.

Whether miRNA Downregulation is Necessary or Sufficient for Initiating EMT.

Enforcing ectopic expression of each microRNA in tet-ind Pez-MDCK cells that are then induced to express Pez (treatment (a) below, is enforced and monitoring of the expression of mesenchymal markers such as Snail, ZEB1, fibronectin, and the epithelial marker E-cadherin is carried out. Similarly, whether ectopic expression of the microRNAs can drive cells that have already undergone EMT back towards an epithelial phenotype (treatment (b)) is measured using the same markers. To what extent relief of microRNA-mediated repression is sufficient to drive epithelial cells towards a mesenchymal phenotype is accessed (treatment (c)), by blocking miRNA function in MDCK cells, and measuring the various markers. Treatments (a) to (c) are performed and cells harvested for RNA and protein after 2, 3 and 4 days. Snail, ZEB1, fibronectin, and E-cadherin expression is measured by qRT-PCR and Western blotting.
a. Tet-ind Pez-MDCK cells are transfected with miRNA oligo precursors (Pre-miR™ miRNA precursor molecules, Ambion) either individually or together, and with and without induction of Pez with doxycyclin.
b. Mesenchymal Pez-MDCK cells are transfected with the Pre-miR™ miRNA precursor molecules or mock transfected.
c. 2′O-methyl RNA antisense to microRNAs has been found to be a potent inhibitor of miRNA function. miRNA function in MDCK cells is blocked, individually and together, by transfecting the cells with 2′O-methyl antisense RNAs or a control 2′O-methyl RNA. To verify the 2′O-methyl RNA blocks function of the cognate microRNA luciferase reporters containing a sequence in the 3′UTR perfectly complementary to each microRNA are constructed.

TABLE 3
Summary of Pez suppression in zebrafish (whole mount in situ
hybridisation)

| | 1^{st} | Peak | Expression |
|---|---|---|---|
| Tissue/Organ | appearance | expression | off |
| Brain (VZ/SVZ) | 20 hpf | 24 hpf | 36 hpf |
| Somites | ND | 20-24 hpf | 24-36 hpf |
| (anterior boundary) | | | |
| Heart | 36 hpf | 42 hpf | 48 hpf |
| Fin (growing tips) | 42 hpf | 48 hpf | ND |

TABLE 4
Some of the defects found in Pez zebrafish morphants.

| Organ | Defects |
|---|---|
| Brain | Lack of distinct boundaries; higher cell densities in |
| | ventricular/sub-ventricular zones; shortened |
| | longitudinal axis |
| Somite | Boundaries form but are irregular and not sharpened |
| Melanocytes | Loss of migration cues |
| Heart | Refluxing between atrium and ventricle (defect in |
| | valve formation); looping defect; pericardial oedema |
| Pectoral fin | Stunting of fins |
| Vascular | Leaky blood vessels |

### Example 3

### Methods

### Cell Culture

MDCK, MDA-MB-231, MCF-7, and MDA-MB-468 cells were maintained in Dulbecco's Modified Eagles Medium (DMEM; Invitrogen, Carlsbad, Calif.) supplemented with 10% fetal bovine serum (FBS). MDA-MB-435 were maintained in Alpha Modified Eagles Medium (αMEM; Invitrogen) supplemented with 5% FBS. The MDCK-Pez and MDCK-vector stable cell lines were generated by stable transfection of the protein tyrosine phosphatase Pez (PTPPez) or empty vector, respectively, into MDCK cells. All of the experiments utilised the MDCK-Pez clone A with the exception of the microarray experiments (detailed below). TGF-β stimulation experiments were performed with a 5 ng/ml concentration of recombinant human TGF-β1 (R&D systems, Minneapolis, Minn.). Unstimulated MDCK cells were split once a week at a 1:10 ratio, whereas TGF-β-treated cells were split twice a week at 1:5 to retain cell viability.

### RNA Extraction and Real-Time PCR

Total RNA was extracted using Trizol (Invitrogen) according to the manufacturer's instructions. For mRNA analysis, complementary DNA (cDNA) was randomly primed from 2.0 μg of total RNA using the Omniscript reverse transcription kit (Qiagen, Hilden, Germany). Real-time PCR was subsequently performed in triplicate with a 1:4 dilution of cDNA using the Quantitect SyBr green PCR system (Qiagen) on a Rotorgene 6000 series PCR machine (Corbett Research, Sydney, Australia). Data was collected and analysed using the Rotorgene software accompanying the PCR machine. Threshold cycle Ct values were determined on auto-threshold settings with reference to a standard dilution curve. All mRNA quantitation data is normalised to GAPDH. For microRNA analysis, real time PCR was performed as above using TaqMan microRNA assays according to the manufacturer's instructions (Applied Biosystems, Foster City, Calif.), or where specified, using locked nucleic acid-mediated real time PCR (Raymond et al., 2005, RNA. 11:1737-1744). All microRNA data is expressed relative to a U6 snRNA TaqMan PCR performed on the same sample.

### MicroRNA Microarray

MicroRNA microarrays were synthesised by spotting of complementary DNA probes to 377 microRNAs (mirVana miRNA probe set v1; Ambion, Austin, Tex.) in quadruplicate onto Corning epoxide coated slides. Samples from Trizol-extracted RNA (20 μg) were enriched for microRNA using the flashPAGE fractionator system (Ambion) and subsequently labelled for hybridisation using the mirVana miRNA labelling kit (Ambion). Three competitive hybridisation experiments were performed in duplicate using enriched microRNA fractions pooled from four independent MDCK-vector clones and eight independent MDCK-Pez clones. Arrays were scanned using a GenePix 4000B Scanner driven by GenePix Pro 4.0 (Molecular Devices, Sunnyvale, Calif.). All analyses were performed in the freely-available statistical programming and graphics environment R (http://cran.r-project.org). Differentially expressed miRNAs were identified using the empirical Bayes approach which ranks genes on a combination of magnitude and consistency of differential expression (Smyth, G. K., 2004 Stat. Appl. Genet. Mol. Biol. 3, Article 3).

### ZEB1 and SIP1 3′UTR Reporter Analysis

The 3′UTR's of ZEB1 and SIP1 were amplified by PCR from HEK-293 genomic DNA and cloned into the XbaI site downstream of Renilla luciferase (RL) in a CMV-driven RL reporter (pCI-neo-hRL30). RL reporter plasmids (6 μM) and pGL3 control (500 ng for normalisation; Promega, Madison, Wis.) were transfected with Lipofectamine 2000 (Invitrogen) into MDCK and MDCK-Pez cells seeded in 24-well plates (6×10⁴ cells/well). The total amount of DNA in each transfection was made up to 1.0 μg with the unrelated pBS-SK Bluescript (+) plasmid (Stratagene, La Jolla, Calif.). Cells were harvested after 48 h for assay using the Dual Luciferase reporter assay system (Promega). For cotransfection experiments, 4 nM of synthetic microRNAs (Pre-miR, Ambion) or 30 nM microRNA inhibitor (Anti-miR, Ambion) were added to the above reactions. All experiments were performed in triplicate with data pooled from at least three independent experiments.

Transfection of microRNA Precursors and Inhibitors

MDCK-Pez cells were seeded at 6×10⁴ cells/well in 24-well plates and transfected with a 60 nM final concentration of synthetic microRNAs (20 nM of each of miR-200a, miR-200b and miR-205 Pre-miRs, Ambion) using HiPerFect transfection reagent (Qiagen). Total RNA and protein was harvested for assay three days post-transfection. MDCK cells were seeded at 2×10⁴ cells/well in 24-well plates and transfected with a 300 nM final concentration of microRNA inhibitors (100 nM of each of miR-200a, miR-200b and miR-205 Anti-miRs, Ambion) as above. Following three days of transfection, cells were split, and re-transfected with additional Anti-miRs, with this process repeated for up to a total of nine days. Total RNA was harvested from cells at six or nine days post-transfection.

### Western Blotting

Extracts were prepared from transfected cells by Triton X-100 lysis (50 mM Hepes, pH 7.5, 150 mM sodium chloride, 10 mM sodium pyrophosphate, 5 mM EDTA, 50 mM sodium fluoride, 1% Triton X-100 with protease inhibitor cocktail) and 50 μg fractionated on a 7.5% SDS polyacrylamide gel. After transfer onto a nitrocellulose membrane, probing was carried with ZEB1-(ZEB E-20; Santa Cruz Biotechnology, Santa Cruz, Calif.) or tubulin—(Abeam, Cambridge, UK) specific antibodies. Membranes were exposed using the ECL method (GE Healthcare, Sydney, Australia) according to the manufacturer's instructions.

### Fluorescent Staining for E-cadherin and F-actin

MDCK and MDCK-Pez cells were transfected with microRNA precursors or inhibitors as above, plated into chamber slides (BD Biosciences, Bedford, Mass.) and left for nine or three days. For E-cadherin staining, cells were fixed in 4% paraformaldehyde, permeabilised in 0.1% Triton X-100, and probed with mouse-anti-E-cadherin antibody (Transduction Laboratories, Lexington, Ky.). The primary antibody was detected using a goat-anti-mouse-Alexa 594 conjugated antibody (Invitrogen). To visualise nuclei, cells were co-stained with 4′-6-Diamidino-2-phenylindole (DAPI; Invitrogen). For F-actin staining, fixed and permeabilised cells were incubated with rhodamine phalloidin (Invitrogen) for 10 min. Cells were visualised on an Olympus IX81 microscope and pictures were taken using a Hamamatsu Orca camera. Images were analysed with Olympus Cell^{R} software.

### Migration Assays

Migration assays were performed in triplicate using Transwell migration chambers (8 μm pore size; Costar, Cambridge, Mass.) coated with 3.5 μg fibronectin on the top and underside of the membrane. MDCK cells transfected with microRNA inhibitors were plated 9 days post-transfection in serum-free media (5×10⁴ cells/Transwell) and allowed to migrate towards a 10% FBS gradient for 4 h. Cells remaining on the top of the filter were scrubbed off and cells that had migrated to the underside of the filter were fixed in methanol and stained with DAPI. Whole filters were manually counted under fluorescence.

### Primer Sequences

Primer sequences used for real-time PCR, and ZEB1 and SIP1 3′UTR cloning are shown in the supplementary information accompanying this paper (Supplementary Information, Table 2).

### Results and Discussion

It was found that miR-205 and all 5 members of the miR-200 family (miR-200a, miR-200b, miR-200c, miR-141 and miR-429) are drastically downregulated in cells that have undergone EMT in response to TGF-β or to ectopic expression of the protein tyrosine phosphatase, PTP-Pez. These microRNAs repress expression of ZEB1 (also known as δEF1) and SIP1 (also known as ZEB2), transcription repressors that regulate E-cadherin expression and are implicated in EMT and tumour metastasis. Downregulation of the microRNAs was sufficient to initiate EMT, while their ectopic expression in mesenchymal cells can initiate MET. Expression of the microRNAs was inversely correlated with invasive and metastatic potential in a panel of human breast cancer cells, indicating their downregulation is an essential step in tumour metastasis.

A search was conducted for microRNAs whose expression changed during EMT. The specific and coordinate downregulation of five related microRNAs (the miR-200 family) and one unrelated microRNA (miR-205) during EMTs provoked by TGF-β or the protein tyrosine phosphatase, PTP-Pez. These microRNAs directly target two transcription factors, ZEB1 and SIP1, which are known to be key instigators of EMT through their repression of the epithelial cell-cell adhesion protein E-cadherin.

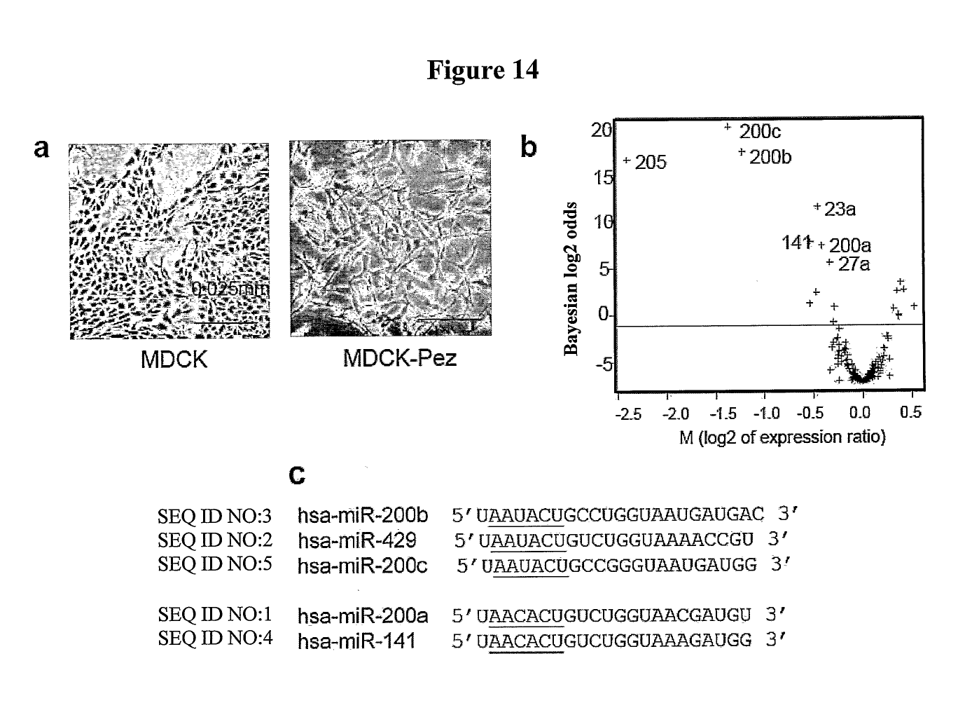

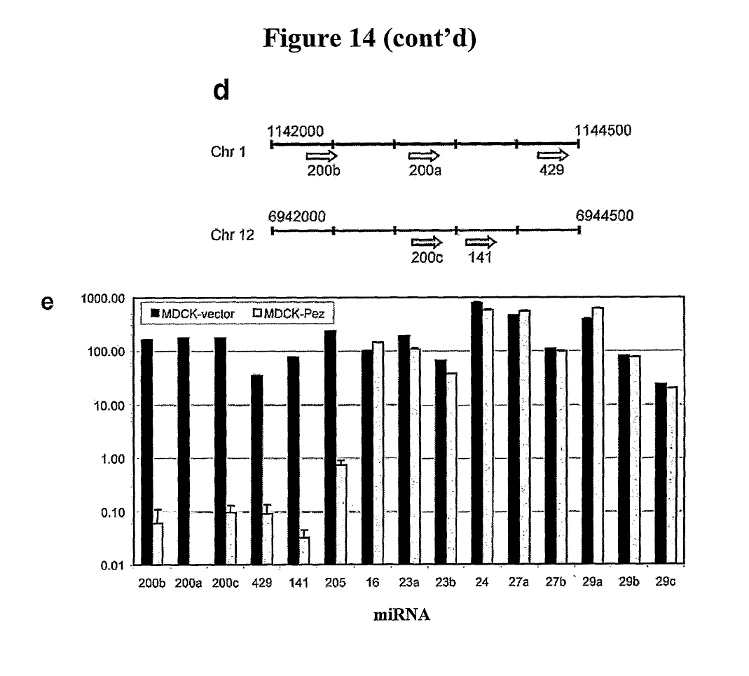

To examine whether microRNAs play a role in EMT, an in vitro model of EMT by stable transfection of MDCK kidney epithelial cells with the protein tyrosine phosphatase Pez (PTP-Pez) was utilised. Over-expression of PTP-Pez causes MDCK cells to undergo an EMT as indicated by loss of E-cadherin expression, gain in expression of the mesenchymal markers fibronectin, ZEB1 and SIP1, loss of cohesion, induction of cell motility, and a change in cell morphology (FIG. 14a). MicroRNA microarrays were used to compare microRNA levels in MDCK and MDCK-Pez cells and it was found that miR-205 and all 5 members of the miR-200 family were strongly downregulated in the MDCKPez cells (FIG. 14b, Table 5). The miR-200 family microRNAs are clustered at two locations in the genome (FIG. 14d), and are also highly related in sequence (FIG. 14c). Using quantitative real time PCR assays it was confirmed that all 5 members of the miR-200 family, as well as miR-205, were downregulated by more that 100-fold in the mesenchymal MDCK-Pez cells, while a selection of other microRNAs were confirmed to be largely unchanged (FIG. 14e).

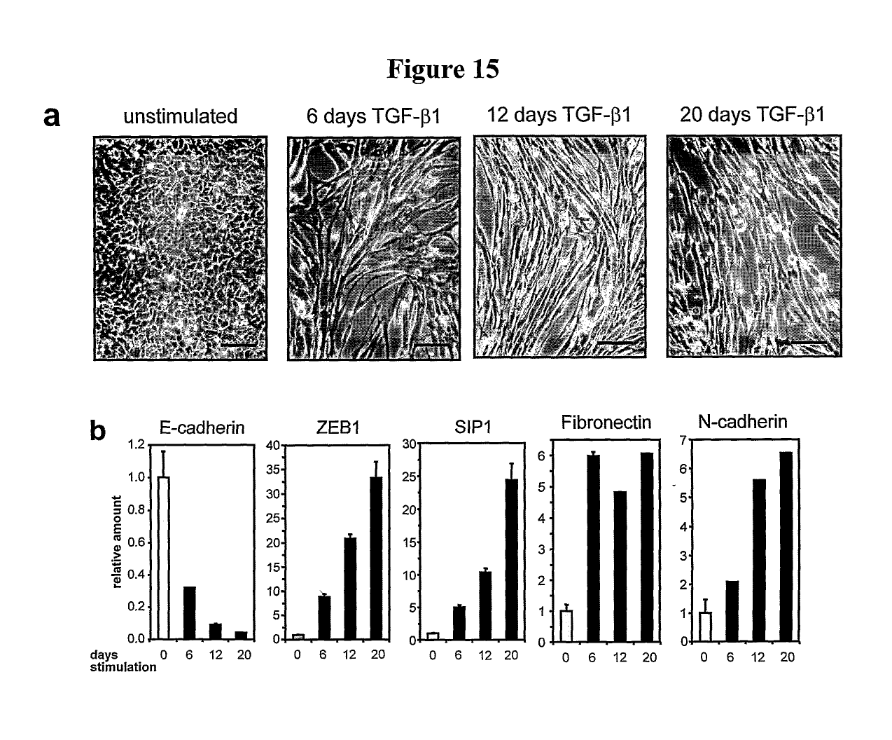

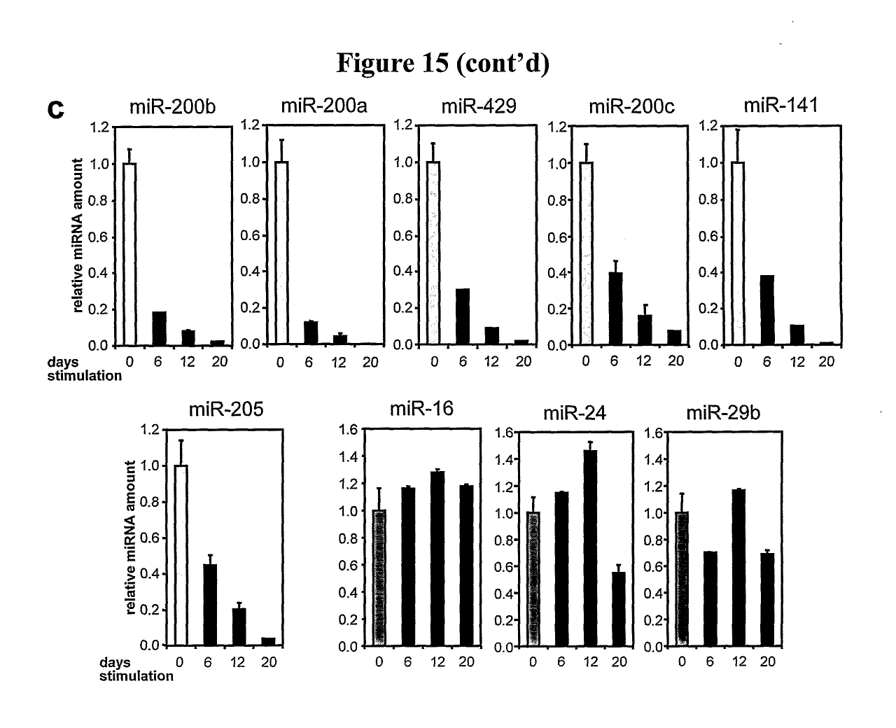

To verify that the downregulation of miR-205 and the miR-200 family are characteristic of EMT, and not an unrelated response to PTP-Pez over-expression, their regulation in cells induced to undergo EMT in response to TGF-13 was examined. MDCK cells treated with TGF-β1 underwent a morphological change (FIG. 15a) accompanied by a loss of cohesion, a decline in the expression of E-cadherin and induction of the mesenchymal markers, fibronectin, N-cadherin, ZEB1 and SIP1 (FIG. 15b). The miR-200 family and miR-205 were selectively downregulated (FIG. 15c), indicating they are involved in TGF-β-induced EMT.

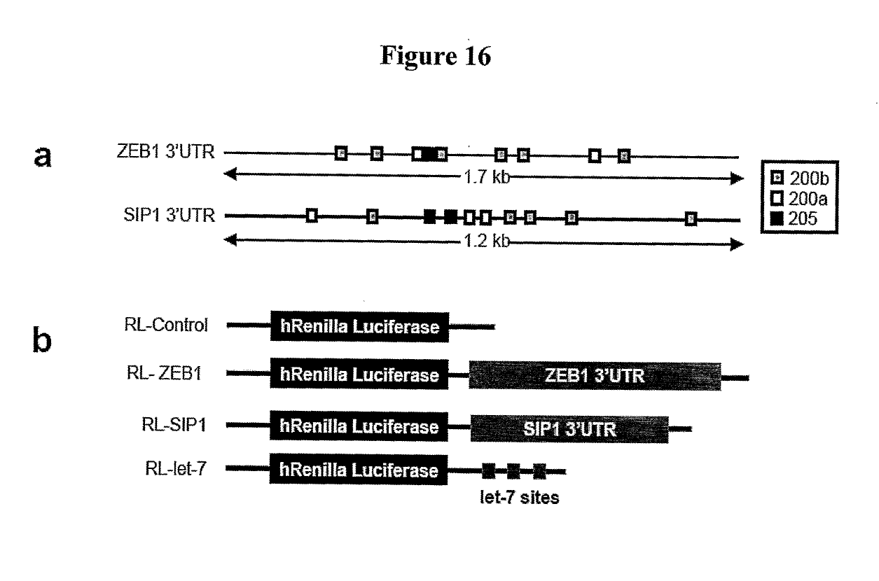

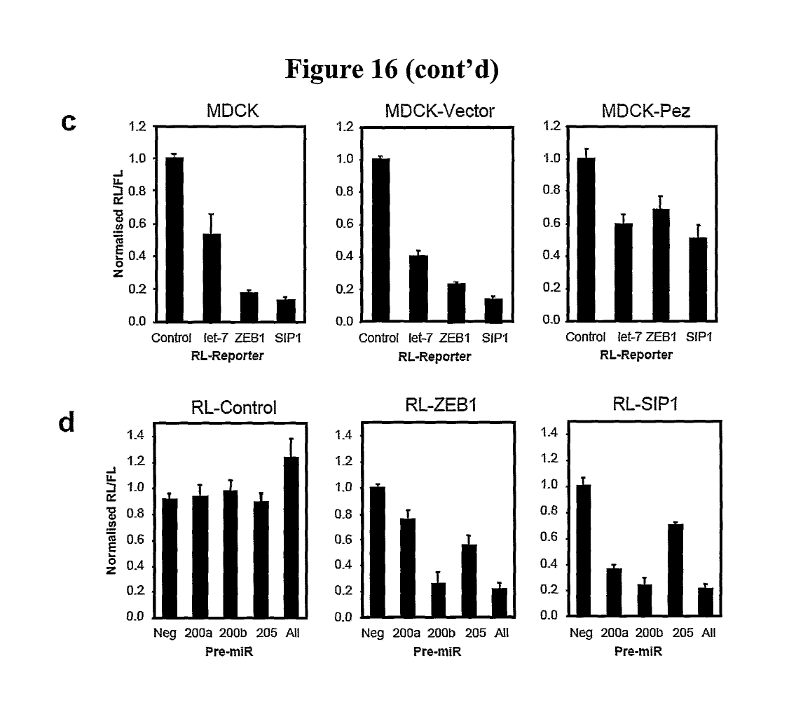

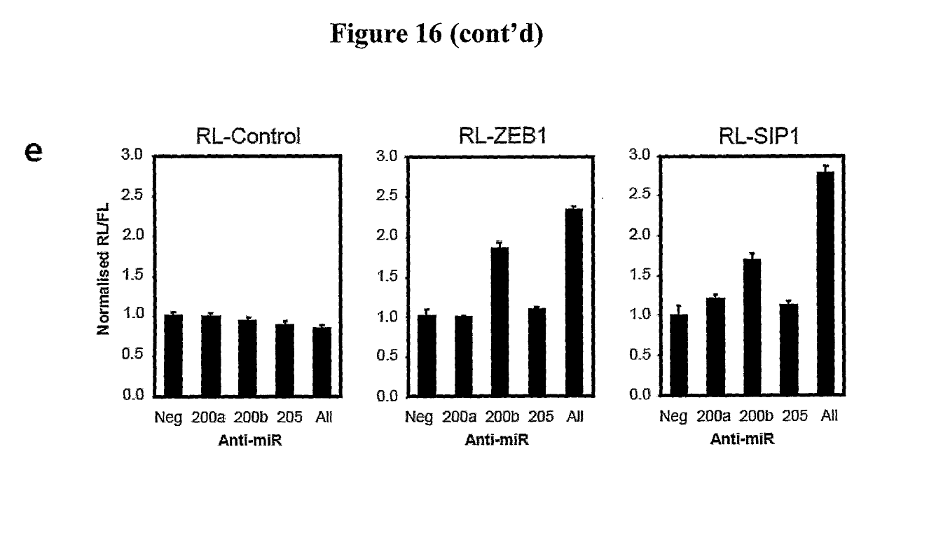

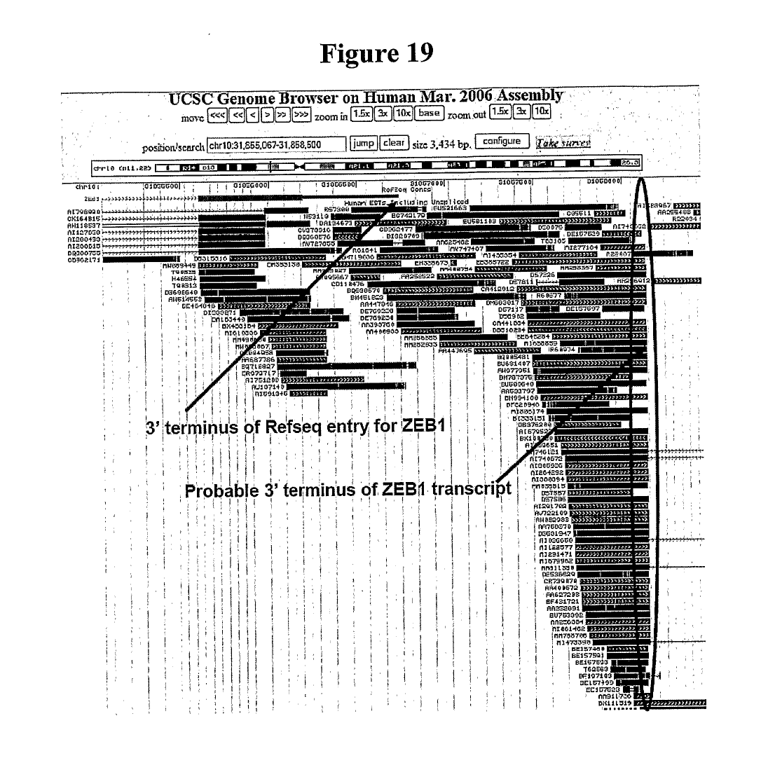

In animals, microRNA function generally involves nucleotides 2 to 7 of the microRNA, commonly called the seed sequence, making uninterrupted base pairing with a complementary sequence in the 3′UTR of the target mRNA. Based on the similarity of their seed sequences, miR-200a, and miR-141 are assumed to interact with the same target sites (hereafter referred to as miR-200a sites), while miR-200b, miR-200c and miR-429 are likely to recognise the same sites (hereafter referred to as miR-200a sites) (FIG. 14c). The target prediction program Targetscan (Lewis, et al. 2005, Cell 120:15-20) indicates that highly conserved miR-200b sites are present in the ZEB1 and SIP1 mRNAs. ZEB1 and SIP1 are repressors of E-cadherin transcription that have been implicated in EMT (Comijn et al. 2001, Mol. Cell. 7:1267-1278; Eger et al. 2005, Oncogene 24, 2375-2385). The SIP1 3′UTR is predicted to contain 3 sites for miR-200a, 5 for miR-200b and 2 for miR-205 (FIG. 16a). Targetscan identifies 2 potential miR-200b sites in the ZEB1 mRNA, but it was noticed that the Genbank Refseq entry for human ZEB1 (NM_{—}030751) is artificially truncated (FIG. 19). Searching the complete ˜1.7 kb 3′UTR by manual inspection revealed that ZEB1 contains 2 putative sites for miR-200a, 5 for miR-200b and 1 for miR-205 (FIG. 16a); significantly all are conserved between human, mouse and dog.

To test whether ZEB1 and SIP1 are targeted by microRNAs, their 3′UTRs were attached to Renilla Luciferase (RL) reporter genes (FIG. 16b) and measured the reporter activity in MDCK cells (which express the microRNAs), and in MDCK-Pez cells (which have very low levels of the microRNAs). Addition of the ZEB1 and SIP1 3′UTRs to the luciferase reporter strongly repressed expression in MDCK cells, but was much less inhibitory in MDCK-Pez cells (FIG. 16c), whereas control reporters were equally expressed in both cell types. These results are consistent with the ZEB1 and SIP1 3′UTRs being targeted by microRNAs in MDCK cells.

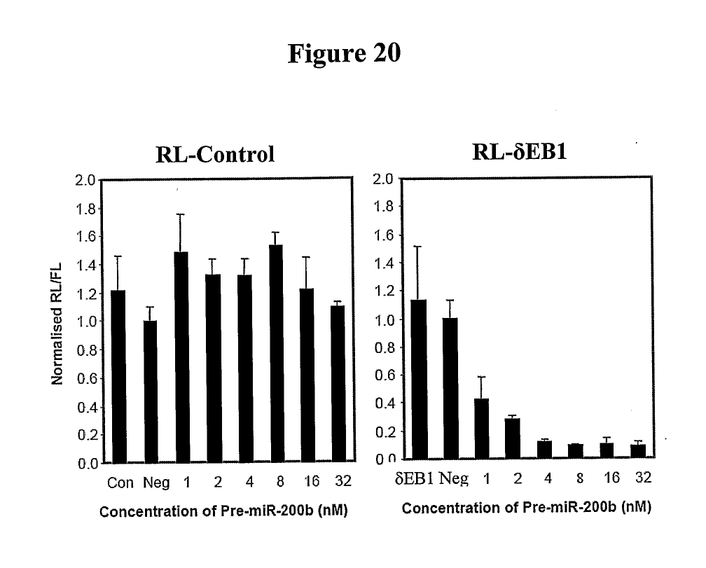

To verify that the miR-200 microRNAs can repress the RL-ZEB1 and RL-SIP1 reporters synthetic microRNA precursors (Pre-miRs, Ambion) were cotransfected with the reporter genes into MDCK-Pez cells. The minimum effective concentration of Pre-miR, determined by titrating the miR-200b Pre-miR, was found to be 4 nM (FIG. 20). Cotransfecting miR-200b had a strong repressive effect on both RL-ZEB1 and RL-SIP1, inhibiting expression by ˜80% (FIG. 16d). MiR-200a inhibited RL-—SIP1 more strongly than it inhibited RL-ZEB1, while miR-205 was mildly inhibitory to both. Thus the effectiveness of each microRNA is roughly in proportion to its number of putative target sites.

To assess the repressive effect of the endogenous microRNAs, reporter activity in MDCK cells in the presence of Anti-miR antisense inhibitors of the microRNAs was measured. Inhibition of miR-200b alone relieved some of the repression afforded by the ZEB1 and SIP1 3′UTRs, but maximal relief of repression was obtained by cotransfecting a combination of the microRNA inhibitors (FIG. 16e). This confirmed that endogenous microRNAs of this family do indeed repress ZEB1 and SIP1, and showed that miR-200a, miR-200b and miR-205 do so in a cooperative manner.

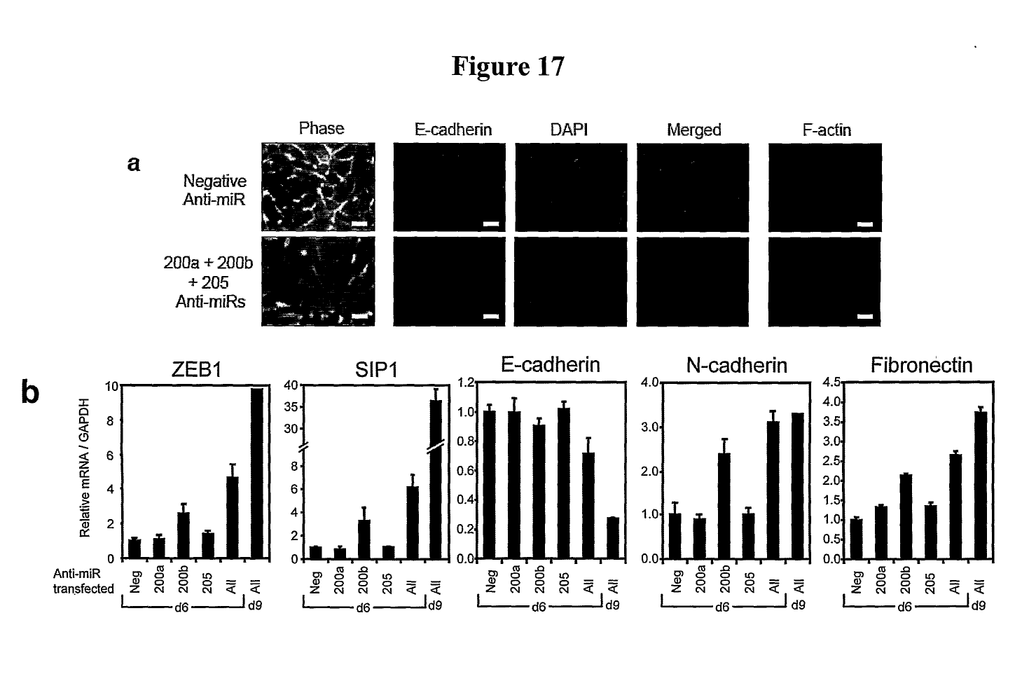

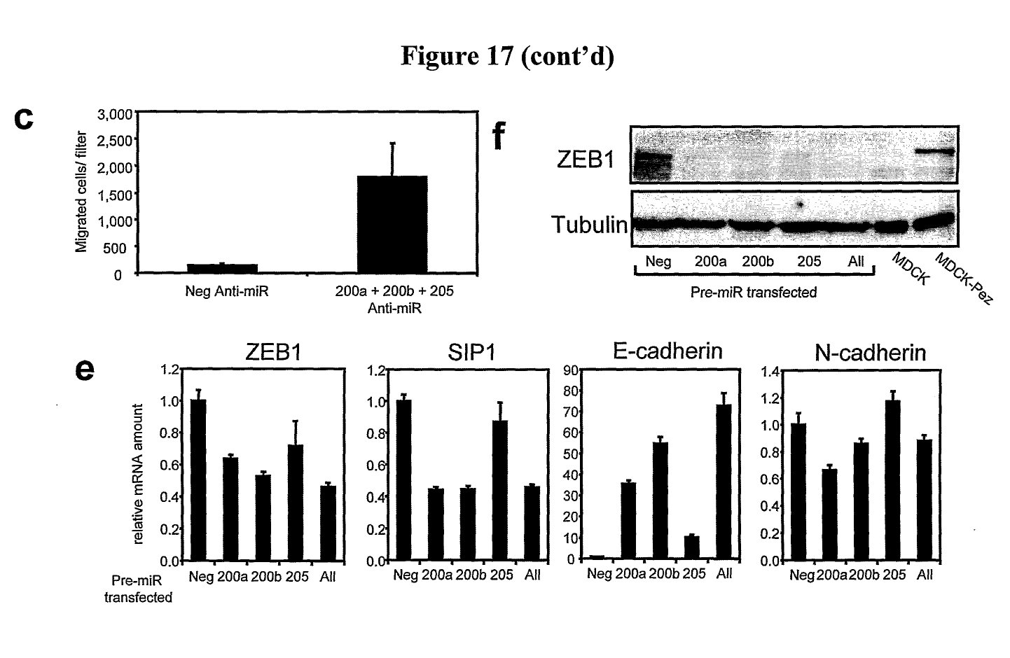

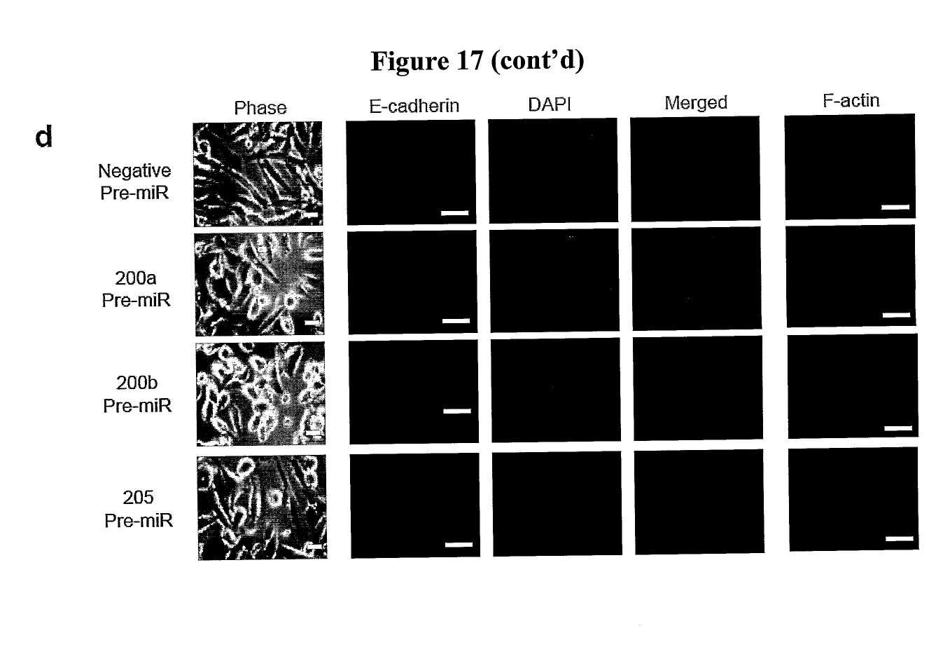

The effect of the microRNA inhibitors on cell phenotype was examined. After nine days of transfection of MDCK cells with anti-miR-200b inhibitor (not shown), or a combination of inhibitors to miR-200a, miR-200b, and miR-205, the cells had begun to adopt a mesenchymal-like morphology (FIG. 17a). The actin cytoskeleton was re-arranged from a cortical to a stress-fibre pattern, E-cadherin was lost from the plasma membrane (FIG. 16a), ZEB1, SIP1, fibronectin and N-cadherin mRNAs were induced and E-cadherin mRNA was reduced (FIG. 17b). The level of induction of ZEB1 and SIP1 mRNAs after inhibition of all three microRNAs was 2-fold greater than with miR-200b alone, indicating the effect of the combination of microRNAs is synergistic. These data mirror the results observed with the RL-ZEB1 and RL-SIP1 reporters (FIG. 16e). Whether this EMT was accompanied by a change in cell motility, using a Transwell migration assay was also investigated. After 4 hours of migration, there was >10-fold increase in the migration of cells where microRNA were inhibited relative to the control (FIG. 16c), further demonstrating these cells had gained functional mesenchymal characteristics.

Having found that miR-200 family expression is necessary for maintenance of the epithelial phenotype, whether ectopic expression of the microRNAs in mesenchymal cells would promote mesenchymal-epithelial transition (MET), the reverse of EMT was investigated. Transfection of MDCK-Pez cells with miR-200a, miR-200b or miR-205 (Pre-miRs) caused the cells to undergo a morphological change from the spindle-shaped mesenchymal form to a rounded epithelial-like form, with many cells aggregating together in groups (FIG. 17d). Immunofluorescent staining of these cells for E-cadherin revealed expression was induced and localised to the plasma membrane, typical of the pattern observed in epithelial cells (FIG. 17d). The degree of E-cadherin induction in the cells correlated with the ability of each microRNA to upregulate E-cadherin mRNA levels (FIG. 17e). Examination of the F-actin distribution in these cells revealed a re-arrangement of the actin cytoskeleton from a stress-fibre to a cortical pattern (FIG. 17d). Collectively, these changes are indicative of the cells having reverted from a mesenchymal to a more epithelial phenotype. To confirm the epithelial-like reversion in cell morphology was due to downregulation of ZEB1 and SIP1 mRNA and, in the case of ZEB1, protein levels in these samples were measured. Ectopic expression of miR-200a or miR-200b reduced ZEB1 mRNA (FIG. 17e), and even more strongly reduced ZEB1 protein (FIG. 17f), providing further evidence of a direct repression of ZEB1 by the microRNAs. SIP1 mRNA levels were similarly reduced (FIG. 17e), consistent with this also being directly regulated by the microRNAs, but the lack of suitable antibodies prevented a direct measurement of SIP1 protein. In accordance with the downregulation of ZEB1 and SIP1, a proportional increase in the level of E-cadherin mRNA was observed indicative of their influence on E-cadherin transcription. This increase in E-cadherin was also accompanied by a modest decrease in the mesenchymal marker N-cadherin (FIG. 17e). Taken together, these data indicate that the miR-200 family can induce a MET-like reversion of MDCK-Pez cells.

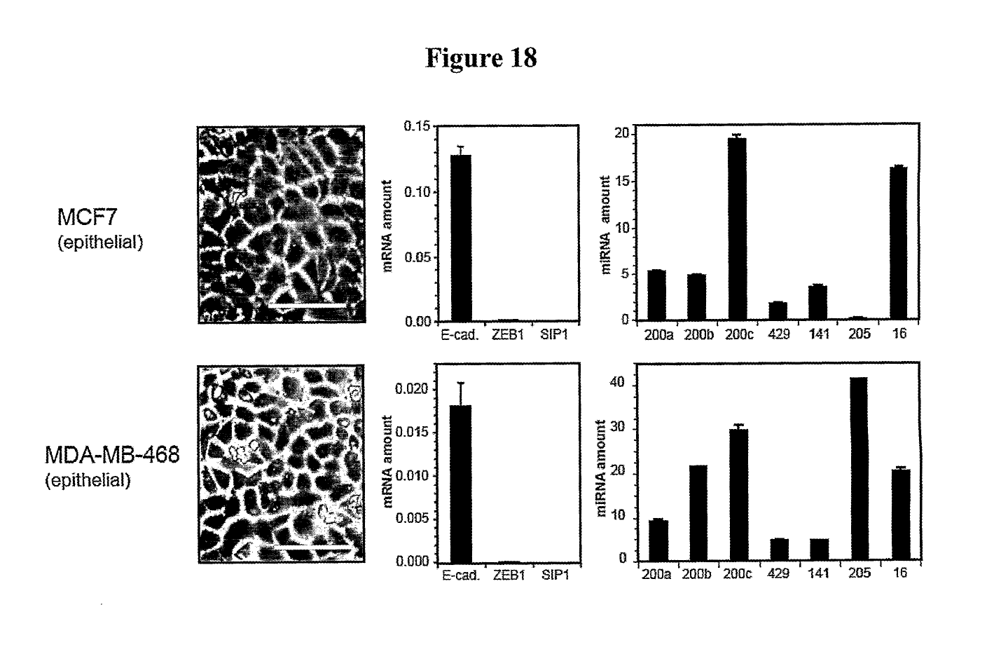

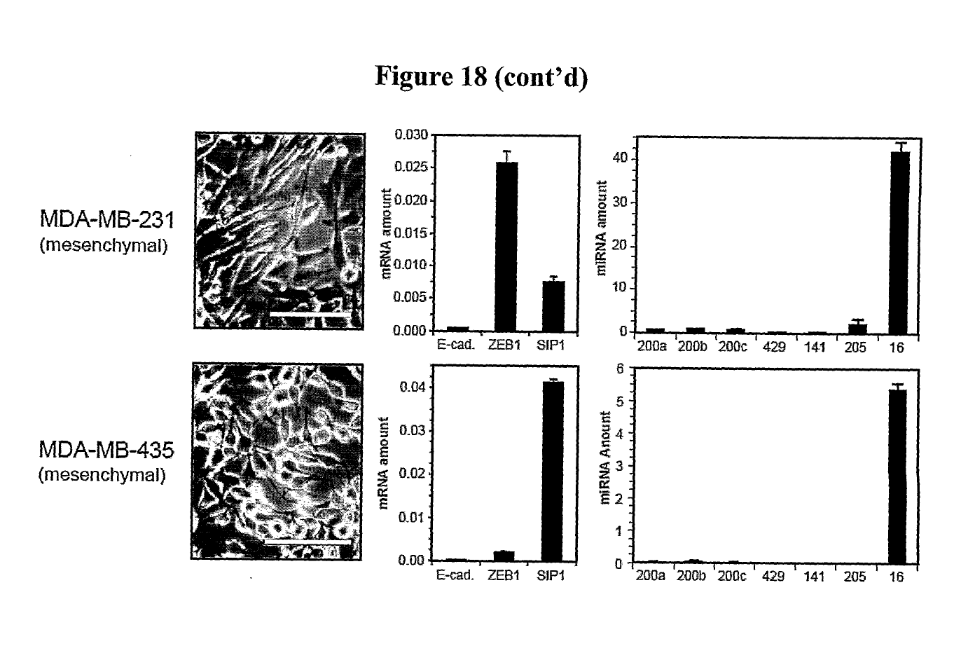

Several studies have implicated a role for EMT in breast cancer metastasis using in vivo mouse model systems (Yang et al., 2004, Cell 117:927-939; Huber et al. 2004, J. Clin. Invest 114:569-581; Moody et al. 2005, Cancer Cell 8:197-209). In addition, the invasiveness of commonly used breast cancer cell lines is often correlated with their mesenchymal state (Lacroix, M. & Leclercq, G., 2004, Breast Cancer Res. Treat. 83:249-289). To investigate whether the regulation of EMT and invasive capacity by the miR-200 family might extend to breast cancer cells, two well-characterised epithelial lines and two well-characterised mesenchymal lines were examined their expression of the miR-200 family and miR-205. MDA-MB-231 and MDA-MB-435 cells, which are invasive and mesenchymal in morphology, expressed low to undetectable amounts of each member of the miR-200 family and miR-205 (FIG. 18). In contrast, the epithelial cell lines, MCF-7 and MDA-MB-468, expressed much higher levels of all members of the miR-200 family. MiR-205 was highly expressed in MDA-MB-468 cells, but not in MCF-7 cells. Consistent with their level of miR-200 family expression, MCF-7 and MDA-MB-468 cells expressed barely detectable levels of both ZEB1 and SIP1 and high levels of E-cadherin, while the opposite was observed in MDA-MB-231 and MDA-MB-435 cells (FIG. 18). These results parallel the expression pattern differences observed between MDCK and MDCK-Pez cells.

The finding that the miR-200 family and miR-205 play an important role in establishing or maintaining the epithelial phenotype are supported by microRNA expression surveys across numerous tissue types and organisms. In humans, miR-200a and miR-200b expression is enriched in tissues where epithelial cell types predominate including the kidney, colon, lung, breast, and small intestine (Thomson et al. 2004, Nat. Methods 1:47-53; Baskerville & Bartel, 2005, RNA. 11:241-247; Lu et al. 2005, Nature 435, 834-838). Similar expression profiles are also observed during zebrafish embryonic development, where these microRNAs are localised to specific epithelial cell types composing the skin, digestive and respiratory systems (Wienholds et al. 2005, Science 309:310-311). In the chick embryo, miR-200a, miR-200b, and miR-205 are among a limited number of microRNAs induced at early stages within the germ layers, which are formed by an EMT during gastrulation (Darnell, et al. 2006 Dev. Dyn. 235:3156-3165). Consistent with the finding that they are downregulated in EMT and target ZEB1 and SIP1, their expression is specifically localised to the endoderm and ectoderm, but largely excluded from the mesoderm, an area where ZEB1 and SIP1 are prominently expressed during embryogenesis (Funahashi et al. 1993, Development 119:433-446; Miyoshi et al. 2006, Dev. Dyn. 235:1941-1952). In a study of skin morphogenesis, all members of the miR-200 family and miR-205 were found to be among the most highly expressed microRNAs in the epidermis, but were low or completely absent in hair follicles despite both tissues being derived from a single epithelial layer (Yi, R. et al. 2006, Nat. Genet. 38:356-362).

In addition to being essential for embryonic development, EMT has also been implicated in the metastasis of tumours from their primary site. A key step in this process involves the downregulation of E-cadherin through mechanisms such as transcriptional repression, promoter hypermethylation, and occasionally direct mutation of the protein (Thiery, J. P. 2002, Nat. Rev. Cancer 2:442-454). ZEB1 and SIP1, along with the transcription factors snail, slug, E47 and twist, are all able to initiate EMT through binding to E-boxes within the E-cadherin promoter and repressing its transcription (Peinado et al. 2004, Int. J. Dev. Biol. 48:365-375). The relative contribution of each repressor in tumorigenesis may depend on the cellular context or organism. For example, recent evidence has implicated ZEB1 in particular, in the progression of lung, uterine and colon cancers (Ohira et al. 2003, Proc. Natl. Acad. Sci. U.S.A 100:10429-10434; Spoelstra et al. 2006, Cancer Res. 66:3893-3902; Spaderna et al. 2006, Gastroenterology 131:830-840). In colon cancer, upregulation of ZEB1 selectively occurred within dedifferentiated cells at the invasive front of the tumour and was associated with loss of the basement membrane, EMT, and poor patient survival (Spadema et al. supra), suggestive of a direct role of ZEB1 in metastasis. Based on the findings reported here, and the observation that the miR-200 family microRNAs are highly expressed in human colonic epithelium (AGB, ELP and GJG, unpublished observation), downregulation of the miR-200 family precedes the epithelial dedifferention at the invasive front. Furthermore, based on the finding of a reciprocal relationship between the miR-200 microRNAs and SIP1 expression in breast cancer cells, along with the observation that expression of SIP1 and slug, but not snail or twist, is inversely correlated with E-cadherin levels in breast cancer cell lines and indicative of a mesenchymal phenotype (Lombaerts et al. 2006, Br. J. Cancer 94:661-671), downregulation of the miR-200 family is an essential early step in breast cancer metastasis.

Those skilled in the art will appreciate that the invention described herein is susceptible to variations and modifications other than those specifically described. It is to be understood that the invention includes all such variations and modifications. The invention also includes all of the steps, features, compositions and agents referred to or indicated in this specification, individually or collectively, and any and all combinations of any two or more of said steps or features.

TABLE 5
Top 20 most regulated miRNAs between MDCK and MDCK-Pez cells

| Name | Log Ratio | Baysian Log Odds |
|---|---|---|
| hsa_miR_200c | −1.37 | 19.94 |
| hsa_miR_200b | −1.22 | 17.32 |
| hsa_miR_205 | −2.40 | 16.46 |
| hsa_miR_23a | −0.46 | 11.46 |
| hsa_miR_141 | −0.53 | 7.69 |
| hsa_miR_200a | −0.41 | 7.36 |
| hsa_miR_27a | −0.32 | 5.54 |
| hsa_miR_181b | 0.40 | 3.43 |
| hsa_miR_181a | 0.43 | 2.58 |
| hsa_miR_29a | 0.36 | 2.46 |
| mmu_miR_429 | −0.47 | 2.40 |
| hsa_miR_31 | −0.54 | 1.18 |
| hsa_miR_155 | 0.54 | 0.87 |
| hsa_miR_106a | −0.28 | 0.83 |
| hsa_miR_27b | 0.33 | 0.72 |
| hsa_miR_422b | 0.37 | 0.09 |
| hsa_miR_22 | 0.38 | −0.13 |
| hsa_miR_194 | −0.29 | −0.68 |
| hsa_miR_17_5p | −0.24 | −1.41 |
| hsa_miR_422a | 0.26 | −2.21 |

TABLE 6

| | | SEQ ID |
|---|---|---|
| Primer Name | Sequence (5′to 3′) | NO. |
| Real time PCR | | |
| ZEB1 hum/dog for | TTCAAACCCATAGTGGTTGCT | 33 |
| ZEB1 hum/dog rev | TGGGAGATACCAAACCAACTG | 34 |
| SIP1 hum for | CAAGAGGCGCAAACAAGC | 35 |
| SIP1 hum rev | GGTTGGCAATACCGTCATCC | 36 |
| SIP1 dog for | CGGTCCAGAAGAAATGAAGG | 37 |
| SIP1 dog rev | TCCTCAAAGTCTGATGTGCAA | 38 |
| E-cadherin hum for | CCCACCACGTACAAGGGTC | 39 |
| E-cadherin hum rev | CTGGGGTATTGGGGGCATC | 40 |
| E-cadherin dog for | AAGCGGCCTCTACAACTTCA | 41 |
| E-cadherin dog rev | AACTGGGAAATGTGAGCACC | 42 |
| N-cadherin hum/dog for | CAACTTGCCAGAAAACTCCAGG | 43 |
| N-cadherin hum rev | ATGAAACCGGGCTATCTGCTC | 44 |
| N-cadherin dog rev | ATGAAACCGGGCTATCAGCTC | 45 |
| Fibronectin dog for | GCAACTCTGTGGACCAAGG | 46 |
| Fibronectin dog rev | CACTGGCACGAGAGCTTAAA | 47 |
| GAPDH hum for | ACCCAGAAGACTGTGGATGG | 48 |
| GAPDH dog for | CATCACTGCCACCCAGAAG | 49 |
| GAPDH hum/dog rev | CAGTGAGCTTCCCGTTCAG | 50 |
| ZEB1/SIP1 3′UTR | | |
| cloning | | |
| ZEB1 3′UTR for | CAACTAGTCAAAATAAATCCGGGTGTGC | 51 |
| ZEB1 3′UTR rev | TTACTAGTACAGCAGTTCAGGCTTGTTGA | 52 |
| SIP1 3′UTR for | ATACTAGTGGAGTTGGAGCTGGGTATTG | 53 |
| SIP1 3′UTR rev | ACACTAGTTGGAATCAGGATCAGTTGAGAA | 54 |

### BIBLIOGRAPHY

Altschul et al. Nucl. Acids. Res. 25: 3389, 1997
Ausubel et al. In: Current Protocols in Molecular Biology, John Wiley & Sons Inc. 1994-1998
Bagga, et al. Cell 122:553-563, 2005
Bagga, S. et al. Regulation by let-7 and lin-4 miRNAs results in target mRNA degradation. Cell 122, 553-563 (2005).
Bandyopadhyay and Temin, Mol Cell Biol 4:749-754, 1984
Baskerville, S. & Bartel, D. P. Microarray profiling of microRNAs reveals frequent coexpression with neighboring miRNAs and host genes. RNA. 11, 241-247 (2005).
Berglund et al, Biotechnology 11:916-920, 1993
Berkner et al, BioTechniques 6:616-629, 1988
Berkner, Curr Top Microbiol Immunol 158:39-66, 1992
Bonner and Laskey, Eur. J. Biochem. 46: 83, 1974
Boyer et al. Biochem Pharmacol 60:1099, 2000
Brazma and Vilo, FEBS Lett 480:17-24, 2000
Breakefield and Geller, Mol Neurobiol 1:339-371, 1987
Buchschacher and Panganiban, J Virol 66:2731-2739, 1982
Carulli et al. J Cell Biochem Suppl 31:286-296, 1998
Celis et al. FEBS Lett 480:2-16, 2000
Chen et al. Nat. Genet. 38:228-233, 2006
Chen, J. F. et al. The role of microRNA-1 and microRNA-133 in skeletal muscle proliferation and differentiation. Nat. Genet. 38, 228-233 (2006).
Comijn et al. Mol. Cell. 7:1267-1278, 2001
Comijn, J. et al. The two-handed E box binding zinc finger protein SIP1 downregulates E-cadherin and induces invasion. Mol. Cell. 7, 1267-1278 (2001).
Darnell, D. K. et al. MicroRNA expression during chick embryo development. Dev. Dyn. 235, 3156-3165 (2006).
Eger et al. Oncogene 24:2375-2385, 2005
Eger, A. et al. DeltaEF1 is a transcriptional repressor of E-cadherin and regulates epithelial plasticity in breast cancer cells. Oncogene 24, 2375-2385 (2005).
Elbashir et al. Genes Dev 15:188-200, 2001
Elbashir et al. Nature 411:494-498, 2001
Englisch et al. Angewandte Chemie, International Edition, 30: 613, 1991
Fan Kindrey Int 56:1455-1467, 1999
Fazi et al. Cell 123:819-831, 2005
Fazi, F. et al. A minicircuitry comprised of microRNA-223 and transcription factors NFI-A and C/EBPalpha regulates human granulopoiesis. Cell 123, 819-831 (2005).
Fink et al, Ann Rev Neurosci 19:265-287, 1996
Fink et al, Hum Gene Ther 3:1-19, 1992
Fire et al. Nature 391:806-811, 1998
Freese et al, Biochem Pharmaco 40:2189-2199, 1990
Fuchs et al. Anal Biochem 286:91-98, 2000
Funahashi, J., Sekido, R., Murai, K., Kamachi, Y. & Kondoh, H. Delta-crystallin enhancer binding protein delta EF1 is a zinc finger-homeodomain protein implicated in postgastrulation embryogenesis. Development 119, 433-446 (1993).

Going and Gusterson, Eur J Cancer 35:1895-1904, 1999
Gorziglia and Kapikian, J Virol 66:4407-4412, 1992
Helseth et al, J Virol 64:2416-2420, 1990
Huber, M. A. et al. NF-kappaB is essential for epithelial-mesenchymal transition and metastasis in a model of breast cancer progression. J. Clin. Invest 114, 569-581 (2004).
International Patent Application PCT/US92/09196
Iwano et al. J Clin Invest 110:341-350, 2002
Janda et al. J Cell Biol 156:299-313, 2002
Johnson et al, J Virol 66:2952-2965, 1992
Jungblut et al. Electrophoresis 20:2100-2110, 1999
Jurecic and Belmont, Curr Opin Microbiol 3: 316-321, 2000)
Kalluri and Nelson J Clin Invest 112(12):1776-1784, 2003
Kiermer et al. Oncogene 20:6679-6688, 2001
Krützfeldt et al Nature Letters 438:685-1784, 2005
Lacroix, M. & Leclercq, G. Relevance of breast cancer cell lines as models for breast tumours: an update. Breast Cancer Res. Treat. 83, 249-289 (2004).
Larson et al. Cytometry 41:203-208, 2000
Larsson et al. J Biotechnol 80:143-157, 2000
Lewis et al. Cell 120: 15-20, 2005
Lewis, B. P., Burge, C. B. & Bartel, D. P. Conserved seed pairing, often flanked by adenosines, indicates that thousands of human genes are microRNA targets. Cell 120, 15-20 (2005).
Lim et al. Nature 433:769-773, 2005
Lim, L. P. et al. Microarray analysis shows that some microRNAs downregulate large numbers of target mRNAs. Nature 433, 769-773 (2005).
Lombaerts, M. et al. E-cadherin transcriptional downregulation by promoter methylation but not mutation is related to epithelial-to-mesenchymal transition in breast cancer cell lines. Br. J. Cancer 94, 661-671 (2006).
Lu, J. et al. MicroRNA expression profiles classify human cancers. Nature 435, 834-838 (2005).
Madden et al. Drug Discov Today 5:415-425, 2000
Madzak et al, J Gen Virol 73:1533-1536, 1992
Mann and Baltimore, J Virol 54:401-407, 1985
Margolskee, Curr Top Microbiol Immunol 158:67-95, 1992
Marmur and Doty, J. Mol. Biol. 5: 109, 1962
Martin et al. Helv Chim Acta 78:486-504, 1995
Miller et al, J Virol 62:4337-4345, 1988
Miller et al, Mol Cell Biol 5:431-437, 1985
Miller, Curr Top Microbiol Immunol 158:1-24, 1992
Miyoshi, T. et al. Complementary expression pattern of Zfhx1 genes Sip1 and deltaEF1 in the mouse embryo and their genetic interaction revealed by compound mutants. Dev. Dyn. 235, 1941-1952 (2006).
Montgomery et al. Proc Natl Acad Sci USA 95:15502-15507, 1998
Moody, S. E. et al. The transcriptional repressor Snail promotes mammary tumor recurrence. Cancer Cell 8, 197-209 (2005).
Moss, Curr Top Microbiol Immunol 158: 5-38, 1992
Moss, Proc Natl Acad Sci USA 93:11341-11348, 1996
Muzyczka, Curr Top Microbiol Immunol 158:97-129, 1992
Naldini et al, Science 272:263-267, 1996
Nielsen et al. Science 254:1497-1500, 1991
Nieto Nat Rev Mol Cell Biol 3:155-166, 2002
Ohi et al, Gene 89:279-282, 1990
Ohira, T. et al. WNT7a induces E-cadherin in lung cancer cells. Proc. Natl. Acad. Sci. U. S. A 100, 10429-10434 (2003).
Page et al, J Virol 64:5270-5276, 1990
Peinado, H., Portillo, F. & Cano, A. Transcriptional regulation of cadherins during development and carcinogenesis. Int. J. Dev. Biol. 48, 365-375 (2004).
Petropoulos et al, J Virol 66:3391-3397, 1992
Pillai, R. S. et al. Inhibition of translational initiation by Let-7 MicroRNA in human cells. Science 309, 1573-1576 (2005).
Prashar and Weissman, Methods Enzymol 303:258-272, 1999
Quantin et al, Proc Natl Acad Sci USA 89:2581-2584, 1992
Raymond, C. K., Roberts, B. S., Garrett-Engele, P., Lim, L. P. & Johnson, J. M. Simple, quantitative primer-extension PCR assay for direct monitoring of microRNAs and short-interfering RNAs. RNA. 11, 1737-1744 (2005).
Rosenfeld et al, Cell 68:143-155, 1992
Russell and Hirata, Nat Genetics 18:323-328, 1998
Sanghvi, Y. S., Chapter 15, Anti-sense Research and Applications, pages 289-302, Crooke, S. T. and Lebleu, B., ed., CRC Press, 1993
Schneider et al, Nat Genetics 18:180-183, 1998
Shimada et al, J Clin Invest 88:1043-1047, 1991
Slack Science 287:1431-1433, 2000
Smyth, G. K. Linear models and empirical bayes methods for assessing differential expression in microarray experiments. Stat. Appl. Genet. Mol. Biol. 3, Article3 (2004).
Sorge et al, Mol Cell Biol 4:1730-1737, 1984
Spaderna, S. et al. A transient, EMT-linked loss of basement membranes indicates metastasis and poor survival in colorectal cancer. Gastroenterology 131, 830-840 (2006).
Spoelstra, N. S. et al. The transcription factor ZEB1 is aberrantly expressed in aggressive uterine cancers. Cancer Res. 66, 3893-3902 (2006).
Stratford-Perricaudet et al, Hum Gene Ther 1:241-256, 1990
Strutz et al. J Cell Biol 130:393-405, 1995
Sutcliffe et al. Proc Natl Acad Sci USA 97:1976-1981, 2000
Tabara et al. Science 282:430-431, 1998
The Concise Encyclopedia Of Polymer Science And Engineering, pages 858-859,
Kroschwitz, J. I., ed. John Wiley & Sons, 1990
Thiery, J. P. Epithelial-mesenchymal transitions in tumour progression. Nat. Rev. Cancer 2, 442-454 (2002).
Thiery. Curr. Opin. Cell Biol. 15:740-746, 2003
Thomson, J. M., Parker, J., Perou, C. M. & Hammond, S. M. A custom microarray platform for analysis of microRNA gene expression. Nat. Methods 1, 47-53 (2004).
Tijsterman et al. Science 295:694-697, 2002
Timmons and Fire, Nature 395:854, 1998
Timmons et al. Gene 263:103-112, 2001
To, Comb Chem High Throughput Screen 3:235-241, 2000
Tuschl et al. Genes Dev 13:3191-3197, 1999
U.S. patent application Ser. No. 09/334,130
U.S. Pat. No. 3,687,808
U.S. Pat. No. 4,469,863
U.S. Pat. No. 4,476,301
U.S. Pat. No. 4,587,044
U.S. Pat. No. 4,605,735
U.S. Pat. No. 4,667,025
U.S. Pat. No. 4,762,779
U.S. Pat. No. 4,789,737
U.S. Pat. No. 4,824,941
U.S. Pat. No. 4,828,979
U.S. Pat. No. 4,835,263
U.S. Pat. No. 4,845,205
U.S. Pat. No. 4,876,335
U.S. Pat. No. 4,904,582
U.S. Pat. No. 4,948,882
U.S. Pat. No. 4,958,013
U.S. Pat. No. 4,981,957
U.S. Pat. No. 5,023,243
U.S. Pat. No. 5,034,506
U.S. Pat. No. 5,082,830
U.S. Pat. No. 5,109,124
U.S. Pat. No. 5,112,963
U.S. Pat. No. 5,118,800
U.S. Pat. No. 5,118,802
U.S. Pat. No. 5,130,302
U.S. Pat. No. 5,134,066
U.S. Pat. No. 5,138,045
U.S. Pat. No. 5,166,315
U.S. Pat. No. 5,175,273
U.S. Pat. No. 5,177,196
U.S. Pat. No. 5,185,444
U.S. Pat. No. 5,188,897
U.S. Pat. No. 5,194,599
U.S. Pat. No. 5,214,134
U.S. Pat. No. 5,214,136
U.S. Pat. No. 5,216,141
U.S. Pat. No. 5,218,105
U.S. Pat. No. 5,227,170
U.S. Pat. No. 5,235,033
U.S. Pat. No. 5,245,022
U.S. Pat. No. 5,254,469
U.S. Pat. No. 5,258,506
U.S. Pat. No. 5,262,536
U.S. Pat. No. 5,264,423
U.S. Pat. No. 5,264,562
U.S. Pat. No. 5,264,564
U.S. Pat. No. 5,272,250
U.S. Pat. No. 5,276,019
U.S. Pat. No. 5,278,302
U.S. Pat. No. 5,286,717
U.S. Pat. No. 5,292,873
U.S. Pat. No. 5,317,098
U.S. Pat. No. 5,319,080
U.S. Pat. No. 5,321,131
U.S. Pat. No. 5,359,044
U.S. Pat. No. 5,367,066
U.S. Pat. No. 5,371,241
U.S. Pat. No. 5,391,723
U.S. Pat. No. 5,393,878
U.S. Pat. No. 5,399,676
U.S. Pat. No. 5,405,938
U.S. Pat. No. 5,405,939
U.S. Pat. No. 5,414,077
U.S. Pat. No. 5,416,203
U.S. Pat. No. 5,432,272
U.S. Pat. No. 5,434,257
U.S. Pat. No. 5,446,137
U.S. Pat. No. 5,451,463
U.S. Pat. No. 5,453,496
U.S. Pat. No. 5,455,233
U.S. Pat. No. 5,457,187
U.S. Pat. No. 5,459,255
U.S. Pat. No. 5,466,677
U.S. Pat. No. 5,466,786
U.S. Pat. No. 5,470,967
U.S. Pat. No. 5,476,925
U.S. Pat. No. 5,484,908
U.S. Pat. No. 5,486,603
U.S. Pat. No. 5,489,677
U.S. Pat. No. 5,502,177
U.S. Pat. No. 5,510,475
U.S. Pat. No. 5,512,439
U.S. Pat. No. 5,512,667
U.S. Pat. No. 5,514,785
U.S. Pat. No. 5,519,126
U.S. Pat. No. 5,519,134
U.S. Pat. No. 5,525,465
U.S. Pat. No. 5,525,711
U.S. Pat. No. 5,527,899
U.S. Pat. No. 5,536,821
U.S. Pat. No. 5,539,082
U.S. Pat. No. 5,541,306
U.S. Pat. No. 5,541,307
U.S. Pat. No. 5,541,313
U.S. Pat. No. 5,545,730
U.S. Pat. No. 5,550,111
U.S. Pat. No. 5,552,538
U.S. Pat. No. 5,552,540
U.S. Pat. No. 5,561,225
U.S. Pat. No. 5,563,253
U.S. Pat. No. 5,565,552
U.S. Pat. No. 5,565,555
U.S. Pat. No. 5,567,810
U.S. Pat. No. 5,567,811
U.S. Pat. No. 5,571,799
U.S. Pat. No. 5,574,142
U.S. Pat. No. 5,576,427
U.S. Pat. No. 5,578,717
U.S. Pat. No. 5,578,718
U.S. Pat. No. 5,580,731
U.S. Pat. No. 5,585,481
U.S. Pat. No. 5,587,361
U.S. Pat. No. 5,587,371
U.S. Pat. No. 5,587,469
U.S. Pat. No. 5,591,584
U.S. Pat. No. 5,591,722
U.S. Pat. No. 5,594,121
U.S. Pat. No. 5,595,726
U.S. Pat. No. 5,596,086
U.S. Pat. No. 5,596,091
U.S. Pat. No. 5,597,696
U.S. Pat. No. 5,597,909
U.S. Pat. No. 5,599,923
U.S. Pat. No. 5,599,928
U.S. Pat. No. 5,602,240
U.S. Pat. No. 5,608,046
U.S. Pat. No. 5,610,289
U.S. Pat. No. 5,610,300
U.S. Pat. No. 5,614,617
U.S. Pat. No. 5,618,704
U.S. Pat. No. 5,623,070
U.S. Pat. No. 5,625,050
U.S. Pat. No. 5,627,053
U.S. Pat. No. 5,633,360
U.S. Pat. No. 5,639,873
U.S. Pat. No. 5,645,985
U.S. Pat. No. 5,646,265
U.S. Pat. No. 5,646,269
U.S. Pat. No. 5,658,873
U.S. Pat. No. 5,663,312
U.S. Pat. No. 5,670,633
U.S. Pat. No. 5,672,697
U.S. Pat. No. 5,677,437
U.S. Pat. No. 5,677,439
U.S. Pat. No. 5,681,941
U.S. Pat. No. 5,688,941
U.S. Pat. No. 5,700,920
U.S. Pat. No. 5,714,331
U.S. Pat. No. 5,719,262
U.S. Pat. No. 5,721,218
U.S. Pat. No. 5,750,692
U.S. Pat. No. 5,763,588
U.S. Pat. No. 5,792,608
U.S. Pat. No. 5,792,747
U.S. Pat. No. 5,830,653
U.S. Pat. No. 6,005,096
U.S. Pat. No. 6,287,860
Wienholds, E. et al. MicroRNA expression in zebrafish embryonic development. Science 309, 310-311 (2005).
Wilkinson et al, Nucleic Acids Res 20:233-2239, 1992
WO 98/39352
WO 99/14226
Xue et al. Cancer Res 63:3386-3394, 2003
Yang and Liu Am J Pathol 159:1465-1475, 2001
Yang et al. Cell 117:927-939, 2004
Yang, J. et al. Twist, a master regulator of morphogenesis, plays an essential role in tumor metastasis. Cell 117, 927-939 (2004).
Yi, R. et al. Morphogenesis in skin is governed by discrete sets of differentially expressed microRNAs. Nat. Genet. 38, 356-362 (2006).
Zavadil and Bottinger. Oncogene 24:5764-5774, 2005
Zavadil, J. & Bottinger, E. P. TGF-beta and epithelial-to-mesenchymal transitions. Oncogene 24, 5764-5774 (2005).
Zeisberg et al. Am J Pathol 159:1313-1321, 2001

1. A method of modulating EMT, said method comprising contacting an epithelial cell with an agent which either (i) elevates the functional level of an miRNA or family of miRNAs or functional derivatives or fragments thereof or (ii) reduces the functional level of an miRNA or family of miRNAs or functional derivatives or fragments thereof in said cell wherein said miRNAs are differentially expressed in either cell type in tissue undergoing EMT relative to epithelial tissue prior to, during or following EMT and wherein:
(i) upregulating the functional level of an miRNA which is downregulated in mesenchymal cells post EMT relative to epithelial cells, inhibits or downregulates EMT;
(ii) downregulating the functional level of an miRNA which is down-regulated in mesenchymal cells post EMT relative to epithelial cells induces or upregulates EMT;
(iii) upregulating the functional level of an miRNA which is upregulated in mesenchymal cells post EMT relative to epithelial cell induces or upregulates EMT; and
(iv) downregulating the functional level of an miRNA which is upregulated in mesenchymal cells post EMT relative to epithelial cells inhibits or downregulates EMT.

2. A method for modulating MET, said method comprising contacting a mesenchymal cell an agent which either (i) elevates the functional level of an miRNA or family of miRNAs or functional derivatives or fragments thereof or (ii) reduces the functional level of an miRNA or family of miRNAs or functional derivatives or fragments thereof in said cell wherein said miRNAs are differentially expressed in either cell type in tissue undergoing MET relative to epithelial tissue prior to, during or following MET and wherein:
upregulating the functional level of an miRNA which is downregulated in mesenchymal cells post EMT relative to epithelial cells induces or upregulates MET; and
(ii) downregulating the functional level of an miRNA which is upregulated in mesenchymal cells post EMT relative to epithelial cells induces or upregulates MET.

3. The method according to claim 1 wherein said miRNAs exhibit the consensus nucleotide sequence set forth in SEQ ID NO:19:
(SEQ ID NO: 19)
UAAN₁ACUGN₂CN₂CN₃GGUAAN₄N₅N₆N₇G[N₈]ₙ
wherein
N₁ is C or U;
N₂ is C or U;
N₃ is U or G;
N₄ is C, U or A;
N₅ is G or A;
N₆ is A or C;
N₇ is U, A or G;
N₈ is G, A, U, C or G;
n is o or 1.

4. The method according to claim 3 wherein said miRNAs are one or more of the sequences set forth in SEQ ID NOs:1-5.

5. The method according to claim 1 wherein said miRNAs are one or more of the sequences set forth in SEQ ID NOs:6-18.

6. The method according to claim 1 wherein said EMT is down-regulated and the miRNAs of one or more of the sequences set forth in SEQ ID NOs:1-11 or 19 are upregulated.

7. The method according to claim 6 wherein said miRNAs are one or more of the sequences set forth in SEQ ID NOs:1-5.

8. The method according to claim 6 wherein said miRNA is the sequence set forth in SEQ ID NO:6.

9. The method according to claim 1 wherein said EMT is down-regulated and the miRNAs of one or more of the sequences set forth in SEQ ID NOs:12-18 are down-regulated.

10. The method according to claim 1 wherein said EMT is upregulated and the miRNAs of one or more of the sequences set forth in SEQ ID NOs:1-11 or 19 are down-regulated.

11. The method according to claim 10 wherein said miRNAs are one or more of the sequences set forth in SEQ ID NOs:1-5.

12. The method according to claim 10 wherein said miRNA is the sequence set forth in SEQ ID NO:6.

13. The method according to claim 1 wherein said EMT is upregulated and the miRNAs of one or more of the sequences set forth in SEQ ID NOs:12-18 are upregulated.

14. The method according to claim 2 wherein the miRNAs of one or more of the sequences set forth in SEQ ID NOs:1-11 or 19 are down-regulated.

15. The method according to claim 14 wherein said miRNAs are one or more of the sequences set forth in SEQ ID NOs:1-5.

16. The method according to claim 14 wherein said miRNA is the sequence set forth in SEQ ID NOs:6.

17. The method according to claim 2 wherein the miRNAs of one or more of the sequences set forth in SEQ ID NOs:12-18 are down-regulated.

18. The method according to claim 1 wherein said agent is a proteinaceous or non-proteinaceous molecule which binds to and antagonises said miRNA or the recognition motif of said miRNA.

19. The method according to claim 18 wherein said agent is an antibody.

20. The method according to claim 18 wherein said agent is an antisense nucleic acid molecule.

21. The method according to claim 18 wherein said recognition motifs are defined by SEQ ID NOs:20-32.

22. The method according to claim 1 wherein said agent is an isolated miRNA or an RNA oligonucleotide which mimics said miRNA functionality.

23. The method according to claim 22 wherein said RNA oligonucleotide is dsRNA, hairpin dsRNA, siRNA or shRNA.

24. The method according to claim 22 wherein said RNA oligonucleotide comprises a sequence complementary to a recognition motif defined by SEQ ID NOs:20-32.

25. The method according to claim 22 wherein said RNA oligonucleotide is 16, 17, 18, 19, 20, 21, 22, 23, 24, 25, 26, 27, 28 or 29 nucleotides in length.

26. The method according to claim 1 wherein said cell is a cell of the breast, colon, stomach, small intestine, oesophagus, ovary, lung, kidney or prostate.

27. The method according to claim 1 wherein said method is performed in vitro.

28. The method according to claim 1 wherein said method is performed in vivo in a subject.

29. An isolated population of epithelial or mesenchymal cells, which cells have been generated according to the method of claim 1.

30. A method for treating a subject, said method comprising administering to said subject an agent which either (i) elevates the functional level of an miRNA or family of miRNAs or functional fragment or derivative thereof or (ii) reduces the functional level of an miRNA or family of miRNAs or functional fragment or derivative thereof in epithelial or mesenchymal cells, which miRNAs are differentially expressed in either cell type in tissue undergoing EMT relative to epithelial tissue prior to, during or following EMT, and wherein:
(i) upregulating the functional level of an miRNA which is downregulated in mesenchymal cells post EMT relative to epithelial cells, inhibits or downregulates EMT;
(ii) downregulating the functional level of an miRNA which is down-regulated in mesenchymal cells post EMT relative to epithelial cells induces or upregulates EMT;
(iii) upregulating the functional level of an miRNA which is upregulated in mesenchymal cells post EMT relative to epithelial cell induces or upregulates EMT;
(iv) downregulating the functional level of an miRNA which is upregulated in mesenchymal cells post EMT relative to epithelial cells inhibits or downregulates EMT;
(v) upregulating the functional level of an miRNA which is downregulated in mesenchymal cells post EMT relative to epithelial cells induces or upregulates MET; and
(vi) downregulating the functional level of an miRNA which is upregulated in mesenchymal cells post EMT relative to epithelial cells induces or upregulates MET.

31. A method for treating a subject by downregulating or inhibiting EMT, said method comprising administering to said subject an agent which upregulates the functional level of one or more miRNAs or family of miRNAs or functional fragment or derivative thereof wherein said miRNA is upregulated in epithelial cells compared to mesenchymal cells following EMT.

32. A method for treating a subject by upregulating or inducing EMT, said method comprising administering to said subject an agent which downregulates the functional level of one or more miRNAs or family of miRNAs or functional fragment or derivative thereof wherein said miRNA is downregulated in mesenchymal cells compared to epithelial cells after EMT.

33. A method for treating a subject by upregulating MET, said method comprising administering to said subject an agent which upregulates the functional level of one or more miRNAs or family of miRNAs or functional fragment or derivative thereof wherein said miRNA is downregulated in mesenchymal cells compared to epithelial cells after EMT.

34. The method according to claim 30 wherein said miRNAs exhibit the consensus nucleotide sequence set forth in SEQ ID NO:19:
(SEQ ID NO: 19)
UAAN₁ACUGN₂CN₂CN₃GGUAAN₄N₅N₆N₇G[N₈]ₙ
wherein
N₁ is C or U;
N₂ is C or U;
N₃ is U or G;
N₄ is C, U or A;
N₅ is G or A;
N₆ is A or C;
N₇ is U, A or G;
N₈ is G, A, U, C or G;
n is o or 1.

35. The method according to claim 30 wherein said miRNAs are defined by SEQ ID NOs:1-11 or 19.

36. The method according to claim 35 wherein said miRNAs are defined by SEQ ID NOs:1-5.

37. The method according to claim 35 wherein said miRNA is defined by SEQ ID NO 6.

38. A method for treating a subject by downregulating or inhibiting EMT, said method comprising administering to said subject an agent which downregulates the functional level of one or more miRNAs or family of miRNAs or functional fragment or derivative thereof wherein said miRNA is downregulated in epithelial cells compared to mesenchymal cells following EMT.

39. A method for treating a subject by upregulating EMT, said method comprising administering to said subject an agent which upregulates the functional level of one or more miRNAs or family of miRNAs or functional fragment or derivative thereof wherein said miRNA is upregulated in mesenchymal cells compared to epithelial cells following EMT.

40. A method for treating a subject by upregulating MET, said method comprising administering to said subject an agent which downregulates the functional level of one or more miRNAs or family of miRNAs or functional fragment or derivative thereof wherein said miRNA is upregulated in mesenchymal cells compared to epithelial cells following EMT.

41. The method according to claim 38 wherein said miRNAs are defined by SEQ ID NOs:12-18.

42. The method according to claim 32 wherein said agent is a proteinaceous or non-proteinaceous molecule which binds to and antagonises said miRNA or the recognition motif of said miRNA.

43. The method according to claim 42 wherein said agent is an antibody.

44. The method according to claim 42 wherein said agent is an antisense nucleic acid molecule.

45. The method according to claim 42 wherein said recognition motifs are defined by SEQ ID NOs:20-27.

46. The method according to claim 31 wherein said agent is an isolated miRNA or an RNA oligonucleotide which mimics said miRNA functionality.

47. The method according to claim 46 wherein said RNA oligonucleotide is dsRNA, hairpin dsRNA, siRNA or shRNA.

48. The method according to claim 46 wherein said RNA oligonucleotide comprises a sequence complementary to a recognition motif defined by SEQ ID NOs:28-32.

49. The method according to claim 31 wherein said subject is treated for cancer, reducing metastasis, reducing fibrosis, promoting wound healing, modulating organogenesis, regeneration of tissue, reducing disease pathologies associated with diabetic renal nephropathy, allograft dysfunction, cataracts or defects in cardiac valve formation.

50. A method of treating a subject, said method comprising administering to said subject an effective number of the cells of claim 29, or cells differentiated therefrom.

51. The method according to claim 50 wherein said subject is treated for wound repair and is administered to mesenchymal cells treated in accordance with the method of the invention.

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. A animal comprising genetically modified cells comprising genetically modified cells or cells derived from genetically modified cells which express an miRNA or family of miRNAs or which are no longer capable of producing one or more miRNAs or which carry genetic material modified to express or not express an miRNA recognition motif.
